(19) United States
(12) Patent Application Publication
(10) Pub. No.: US 2006/0106102 A1
(43) Pub. Date: May 18, 2006

(54) Napththalene derivatives which inhibit the cytokine or biological activity of microphage migration inhibitory factor (mif)

(75) Inventors: Eric Francis Morand, Victoria (AU); Magdy Naguib Iskander, Victoria (AU); Basil Danylec, Victoria (AU)

Correspondence Address: KING & SPALDING LLP, 1180 PEACHTREE STREET, ATLANTA, GA, 30309, US

(73) Assignee: ERIC FRANCIS MORAND

(21) Appl. No.: 10/517,240

(22) PCT Filed: Jun. 6, 2003
(86) PCT No.: PCT/AU03/00716

§ 371 Date: Oct. 3, 2005

(30) Foreign Application Priority Data
Jun. 7, 2002 (AU) PS 2833
Jun. 7, 2002 (AU) PS2834

(51) Int. Cl. A61K 31/255; A61K 31/235; C07C 69/84; C07C 309/35

(52) U.S. Cl. 514/518; 514/543; 560/64; 560/21; 560/45; 562/81

(57) ABSTRACT

Where Y, R₁-R₈ and R₁₀₁-R₁₀₈ are as defined in the specification. Compounds of formula (II) and methods of inhibiting the cytokine or biological activity of Macrophage Migrating Inhibitory Factor (MIF) comprising contacting MIF with a compound of formula (I) are provided. The invention also relates to methods of treating diseases or conditions where MIF cytokine or biological activity is implicated comprising administration of compounds of formula (1), either alone or as part of a combination therapy.

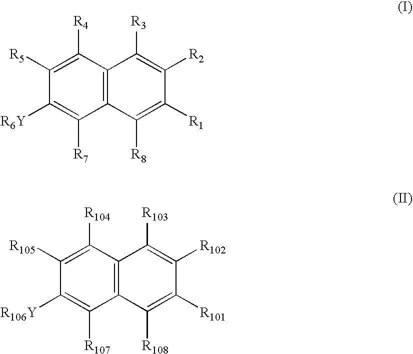

### FIELD OF THE INVENTION

The present invention relates generally to the treatment of diseases or conditions resulting from cellular activation, such as inflammatory or cancerous diseases or conditions. In particular, the invention relates to the use of naphthalene derivatives to inhibit the cytokine or biological activity of macrophage migration inhibitory factor (MIF), and disease or conditions wherein MIF cytokine or biological activity is implicated.

### BACKGROUND OF THE INVENTION

MIF is the first identified T-cell-derived soluble lymphokine. MIF was first described as a soluble factor with the ability to modify the migration of macrophages (1). The molecule responsible for the biological actions ascribed to MIF was identified and cloned in 1989 (2). Initially found to activate macrophages at inflammatory sites, it has been shown to possess pluripotential actions in the immune system. MIF has been shown to be expressed in human diseases which include inflammation, injury, ischaemia or malignancy. MIF also has a unique relationship with glucocorticoids by overriding their anti-inflammatory effects.

Recent studies have indicated that monoclonal antibody antagonism of MIF may be useful in the treatment of sepsis, certain types of cancers and delayed type hypersensitivity. Antibody antagonism of MIF has also been shown to have activity in adjuvant- or collagen-induced arthritis animal models and other models of inflammatory and immune diseases.

Although antibody antagonism of MIF is one potential way to provide therapeutic treatments, such biological molecules can be expensive to prepare on a commercial basis and further, can be limited in the way they are administered (generally by injection) and do not readily lend themselves to formulations for administration by other means eg oral administration.

Small molecule inhibitors may overcome one or more such difficulties connected with the use of biological therapeutic treatments. There exists a need, therefore, for small molecule inhibitors of the cytokine or biological activity of MIF. Small molecule inhibitors of the MIF would have therapeutic effects in a broad range of diseases, whether given alone or in combination with other therapies.

Examples of agents which could be used in combination with a compound of formula (1) include glucocorticoids, antirheumatic drugs, immunosuppressive drugs, anti-cytokine therapies, antagonists or inhibitors of nitrogen-activated protein (MAP) kinases, antagonists or inhibitors of nuclear factor kappa-B (NF-κB) signal transduction pathway, antibodies, protein therapeutics or small molecule therapeutics interacting with adhesion molecules and co-stimulatory molecules, bronchodilators, antagonists of eicosanoid synthesis pathways, agents used for the treatment of inflammatory bowel disease, anti-cancer drugs, antisense olionucleotides, interfering RNA and ribozymes.

For example, glucocorticoids have been used to treat human diseases for over fifty years and are effective in a range of diseases which include inflammation, injury, ischaemia or malignancy. Although debate continues in relation to their impact on disease prognosis, their influence on symptoms and signs of inflammation, especially in the short term, can be dramatic.

Despite their benefits and efficacy, the use of glucocorticoids is limited by universal, predictable, dose-dependent toxicity. Mimicking Cushing's disease, a disease wherein the adrenal glands produce excess endogenous glucocorticoids, glucocorticoid treatment is associated with side effects including immunosuppression (resulting in increased susceptibility to infections), weight gain, change in body habitus, hypertension, oedema, diabetes mellitus, cataracts, osteoporosis, poor wound healing, thinning of the skin, vascular fragility, hirsutism and other features of masculinization (in females). In children, growth retardation is also noted. These side effects are known as Cushingoid side effects.

Since the side effects of glucocorticoids are dose dependent, attempts to reduce the dosage requirement have been investigated, including combination therapies in which glucocorticoids are administered with other therapeutic agents. These combination therapies are sometimes referred to as “steroid-sparing” therapies. However, currently available combination therapies are non-specific as the other therapeutic agents do not address biological events which inhibit the effectiveness of glucocorticoids. Such combination therapies are also typically associated with serious side effects.

Furthermore, glucocorticoids are incompletely effective in a number of disease settings, leading to the concept of “steroid-resistant” diseases. Agents which amplify or enhance the effects of glucocorticoids would not only allow the reduction of dose of these agents but may also potentially render “steroid-resistant” diseases steroid-sensitive.

There is a need for effective therapies which enable a reduction in the dosage level of glucocorticoids. There is also a need for effective treatment of “steroid-resistant” diseases. Preferably, such therapies or treatments would address factors which directly limit the effectiveness of glucocorticoids.

Therapeutic antagonism of MIF may provide “steroid-sparing” effects or be therapeutic in “steroid-resistant” diseases. Unlike other pro-inflammatory molecules, such as cytokines, the expression and/or release of MIF can be induced by glucocorticoids (3), (4). Moreover, MIF is able to directly antagonize the effects of glucocorticoids. This has been shown to be the case for macrophage TNF, IL-1β, IL-6 and IL-8 secretion (5), (6), and for T cell proliferation and IL-2 release (7). In vivo, MIF exerts a powerfull glucocorticoid-antagonist effect in models including endotoxic shock and experimental arthritis (5), (8). In the context of an inflammatory or other disease treated with glucocorticoids, then, MIF is expressed but exerts an effect which prevents the glucocorticoid inhibition of inflammation. It can therefore be proposed that therapeutic antagonism of MIF would remove MIF's role in inhibiting the anti-inflammatory effect of glucocorticoids, thereby allowing glucocorticoids to prevail. This would be the first example of true “steroid-sparing” therapy. In support of this hypothesis is the observation that anti-MIF antibody therapy reverses the effect of adrenalectomy in rat adjuvant arthritis (9). By neutralizing the natural glucocorticoid ‘counter-regulator’ effect of MIF, it is envisioned that with MIF antagonism, steroid dosages could be reduced or even eliminated in inflammatory disease, particularly in those diseases that are associated with the glucocorticoid resistance (10), (11). There is a need, therefore, for therapeutic antagonists of the cytokine or biological activity of MIF.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

In a first aspect, the present invention provides a method of inhibiting cytokine or biological activity of MIF comprising contacting MIF with a cytokine or biological. activity inhibiting effective amount of a compound of formula (I), or a pharmaceutically acceptable salt or prodrug thereof

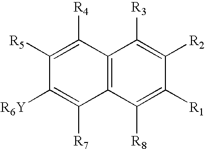

wherein

Y is O, NR₉ or S(O)_{q},

R₁ is selected from hydrogen, C₁₋₆alkyl, —(CR₁₀R_{10′})ₙhalo, —(CR₁₀R_{10′})ₙOR₁₁, —(CR₁₀R_{10′})ₙSR₁₁, —(CR₁₀R_{10′})ₙ—N(R₁₂)₂, —(CR₁₀R_{10′})ₙ—S(O)R₁₁, —(CR₁₀R_{10′})ₙS(O)₂R₁₁, —(CR₁₀R_{10′})ₙ—S(O)₃R₁₁, —(CR₁₀R_{10′})ₙC(O)R₁₃, —(CR₁₀R_{10′})ₙ—C(═NR₁₄)R₁₅ or —(CR₁₀R_{10′})ₙR₁₆;

R₂ is selected from hydrogen, C₁₋₂₀alkyl, C₂₋₂₀alkenyl, C₂₋₂₀alkynyl, —(CR₁₀R_{10′})ₘOR₁₇, —(CR₁₀R_{10′})ₘSR₁₇, —(CR₁₀R_{10′})ₘNR₁₈R₁₉, —(CR₁₀R_{10′})ₘS(O)R₂₀, —(CR₁₀R_{10′})ₘS(O)₂R₂₀, —(CR₁₀R_{10′})ₘC(O)R₂₀, —(CR₁₀R_{10′})ₘC(S)R₂₀, —(CR₁₀R_{10′})ₘC(═NR₁₁)R₁₅ or —(CR₁₀R_{10′})ₘR₁₆;

R₃, R₄ and R₅ are independently selected from hydrogen, C₁₋₃alkyl, —(CR₁₀R_{10′})ₙN(R₁₄)₂, —(CR₁₀R_{10′})ₙOR₁₄, —(CR₁₀R_{10′})ₙSR₁₄ or —(CR₁₀R_{10′})ₙhalo;

R₆ is selected from hydrogen, C₁₋₆alkyl, —C(O)C₁₋₆alkyl, —C(O)N(R₉)₂—, —C(S)N(R₉)₂— or —(CR₁₀R_{10′})ₙR₂₁, or R₆Y and R₅ together may form —X—(CH₂)ₜ-Z- , where X and Z may be independently selected from O, S or NR₁₄;

R₇ and R₈ are independently selected from hydrogen, C₁₋₃alkyl, C₂₋₃alkenyl, C₂₋₃alkynyl or —(CR₁₀R_{10′})ₙR₂₂;

Each R₉ is independently selected from hydrogen or C₁₋₆alkyl;

Each R₁₀ and R_{10′} is independently selected from hydrogen, C₁₋₆alkyl, C₂₋₆alkenyl, C₂₋₆alkynyl, halogen, OR₁₁, SR₁₁, C₁₋₃alkoxy, CO₂R₁₄, N(R₁₄)₂, CN, NO₂, aryl or heterocyclyl;

R₁₁ is hydrogen or C₁₋₆alkyl;

Each R₁₂ is independently selected from hydrogen, C₁₋₆alkyl, C(═NR₁₄)R₁₅, NH—C(═NR₁₄)R₁₅, C(O)R₁₄ or C(S)R₁₄;

R₁₃ is hydrogen, C₁₋₆alkyl, OR₁₄, SR₁₄ or N(R₁₄)₂;

Each R₁₄ is independently selected from hydrogen or C₁₋₃alkyl;

R₁₅ is C₁₋₆alkyl, NH₂, NH(C₁₋₃alkyl) or N(C₁₋₃alkyl)₂, OR₂₃ or SR₂₃;

R₁₆ is hydroxy, C₁₋₃alkoxy, SH, SC₁₋₃alkyl, halo, C(O)R₃₁, C(R₂₄)₃, CN, aryl or heterocyclyl;

R₁₇ is selected from hydrogen, C₁₋₂₀alkyl, C₂₋₂₀alkenyl, C₂₋₂₀alkynyl, (CR₂₆R_{26′})ₛR₂₇, C(O)R₂₅, CO₂R₂₅, C(S)R₂₅, C(S)OR₂₅, S(O)R₂₅, S(O)₂R₂₅, [C(O)CH(R₁₉)NH]ᵣ—R₂₃ or [sugar]ᵣ;

R₁₈ and R₁₉ are independently selected from hydrogen, C₁₋₂₀alkyl, C₂₋₂₀alkenyl, C₂₋₂₀alkynyl, (CR₂₆R_{26′})ₛR₂₇, C(O)R₂₅, C(S)R₂₅, S(O)R₂₅, S(O)₂R₂₅, [C(O)CH(R₂₉)NH]ᵣ—R₂₃, [sugar]ᵣ, C(═NR₂₃)NH₂ or NH—C(═NR₂₃)NH₂;

R₂₀ is selected from hydrogen, C₁₋₂₀alkyl, C₂₋₂₀alkenyl, C₂₋₂₀alkynyl, OR₂₈, SR₂₈, N(R₂₈)₂, [NH—CHR₂₉C(O)]ᵣ—OR₂₃, [sugar]ᵣ, or (CR₂₆R_{26′})ₛR₂₇;

R₂₁, is OR₂₈, SR₂₈, halo or N(R₂₅)₂;

R₂₂ is halo, CO₂H, SO₃H, NO₂, NH₂, CO₂C₁₋₃alkyl, SO₃C₁₋₃alkyl or C(R₂₄)₃;

R₂₃ is hydrogen or C₁₋₃alkyl;

Each R₂₄ is independently selected from hydrogen, Cl or F;

Each R₂₅ is independently selected from hydrogen, C₁₋₂₀alkyl, C₂₋₂₀alkenyl, C₂₋₂₀alkynyl, aryl or (CR₂₆R_{26′})ₛR₂₇;

Each R₂₆ and R_{26′} is independently selected from hydrogen, C₁₋₆alkyl, C₂₋₆alkenyl, C₂₋₆alkynyl, halogen, hydroxy, C₁₋₃alkoxy, SH, C₁₋₃alkylthio, CO₂H, CO₂C₁₋₃alkyl, NH₂, NH(C₁₋₃alkyl), N(C₁₋₃alkyl)₂, CN, NO₂, aryl or heteroaryl;

R₂₇ is hydroxy, C₁₋₆alkoxy, SH, SC₁₋₆alkyl, halo, NH₂, NH(C₁₋₃alkyl), N(C₁₋₃alkyl)₂, C(O)R₃₁, aryl or heterocyclyl;

Each R₂₈ is independently selected from hydrogen, C₁₋₂₀alkyl, C₂₋₂₀alkenyl, C₂₋₂₀alkynyl or (CR₂₆R_{26′})ₛR₃₀;

R₂₉ is the characterising group of an amino acid;

R₃₀ is halogen, hydroxy, C₁₋₃alkoxy, NH₂, NH(C₁₋₃alkyl), N(C₁₋₃alkyl)₂, C(O)R₃₁, aryl or heterocyclyl;

R₃₁ is C₁₋₃alkyl, OH, C₁₋₃alkoxy, aryl, aryloxy, heterocyclyl or heterocyclyloxy;

q is 0, 1, 2 or 3;

n is 0, 1, 2 or 3;

m is 0 or 1 to 20;

r is 1 to 5;

s is 1 to 10; and

t is 1 or 2;

wherein an alkyl, alkenyl, alkynyl, alkyloxy, aryl or heterocyclyl group may be optionally substituted one or more times.

In another aspect, the invention provides a method of treating, preventing or diagnosing a disease or condition wherein MIF cytokine or biological activity is implicated comprising the administration of a treatment, prevention or diagnostic effective amount of a compound of formula (I) or a pharmaceutically acceptable salt or prodrug thereof to a subject in need thereof.

In a further aspect, there is provided the use of a compound of formula (I) or a pharmaceutically acceptable salt or prodrug thereof in the manufacture of a medicament for the treatment, prevention or diagnosis of a disease or condition wherein MIF cytokine or biological activity is implicated.

In particular, the invention provides a method of treating, diagnosing or preventing autoimmune diseases, solid or haemopoeitic tumours, or chronic or acute inflammatory diseases, including a disease or condition selected from the group comprising:
Rheumatic diseases (including but not limited to rheumatoid arthritis, osteoarthritis, psoriatic arthritis, polymyalgia rheumatica) spondyloarthropathies (including but not limited to ankylosing spondylitis, reactive arthritis, Reiter's syndrome), crystal arthropathies (including but not limited to gout, pseudogout, calcium pyrophosphate deposition disease), Lyme disease, connective tissue diseases (including but not limited to systemic lupus erythematosus, systemic sclerosis, polymyositis, dermatomyositis, Sjögren's syndrome), vasculitides (including but not limited to polyarteritis nodosa, Wegener's granulomatosis, Churg-Strauss syndrome), glomerulonephritis, interstitial nephritis, inflammatory bowel disease (including but not limited to ulcerative colitis, Crohn's disease), peptic ulceration, gastritis, oesophagitis, liver disease (including but not limited to cirrhosis, hepatitis), autoimmune diseases (including but not limited to diabetes mellitus, thyroiditis, myasthenia gravis, sclerosing cholangitis, primary biliary cirrhosis), pulmonary diseases (including but not limited to diffuse interstitial lung diseases, pneumoconioses, fibrosing alveolitis, asthma, bronchitis, bronchiectasis, chronic obstructive pulmonary disease, adult respiratory distress syndrome), cancers whether primary or metastatic (including but not limited to colon cancer, lymphoma, lung cancer, melanoma, prostate cancer, breast cancer, stomach cancer, leukemia, cervical cancer, multiple myeloma and metastatic cancer), atherosclerosis (eg ischaemic heart disease, myocardial infarction, stroke, peripheral vascular disease), disorders of the hypothalamic-pituitary-adrenal axis, brain disorders (eg dementia, Alzheimer's disease, multiple sclerosis, demyelinating diseases), corneal disease, iritis, iridocyclitis, cataracts, uveitis, sarcoidosis, diseases characterised by modified angiogenesis (eg diabetic retinopathy, rheumatoid arthritis, cancer), endometrial function (menstruation, implantation, parturition, endometriosis), psoriasis, endotoxic (septic) shock, exotoxic (septic) shock, infective (true septic) shock, other complications of infection, pelvic inflammatory disease, transplant rejection, allergies, allergic rhinitis, bone diseases (eg osteoporosis, Paget's disease), atopic dermatitis, UV(B)-induced dermal cell activation (eg sunburn, skin cancer), malarial complications, diabetes mellitus, pain, inflammatory consequences of trauma or ischaemia, testicular dysfunctions, and wound healing,

comprising the administration of a treatment, diagnosis or prevention effective amount of a compound of formula (I) or a pharmaceutically acceptable salt or prodrug thereof, to a subject in need thereof

A further aspect of the invention provides for the use of a compound of formula (I) or a pharmaceutically acceptable salt or prodrug thereof, in the manufacture of a medicament for the treatment of a disease or condition as above.

A further aspect of the invention provides a pharmaceutical composition comprising a compound of formula (I) or a pharmaceutically acceptable salt or prodrug thereof and a pharmaceutically acceptable carrier, diluent or excipient.

In another aspect, the invention provides a method of treating or preventing a disease or condition wherein MIF cytokine or biological activity is implicated comprising administering to a mammal a compound of formula (I) or a pharmaceutically acceptable salt or prodrug thereof and a second therapeutic agent.

In another aspect, the present invention provides a method of prophylaxis or treatment of a disease or condition for which treatment with a glucocorticoid is indicated, said method comprising administering to a mammal a glucocorticoid and a compound of formula (I) or a pharmaceutically acceptable salt or prodrug thereof.

In yet another aspect, the present invention provides a method of treating steroid-resistant diseases comprising administering to a mammal a glucocorticoid and a compound of formula (I) or a pharmaceutically acceptable salt or prodrug thereof

In a further aspect, the present invention provides a method of enhancing the effect of a glucocorticoid in mammals comprising administering a compound of formula (I) or a pharmaceutically acceptable salt or prodrug thereof, simultaneously, separately or sequentially with said glucocorticoid.

In yet a further aspect, the present invention provides a pharmaceutical composition comprising a glucocorticoid and a compound of formula (I) or a pharmaceutically acceptable salt or prodrug thereof.

In a further aspect of the invention there is provided a use of a glucocorticoid in the manufacture of a medicament for administration with a compound of formula (I) or a pharmaceutically acceptable salt or prodrug thereof for the treatment or prophylaxis of a disease or condition for which treatment with a glucocorticoid is indicated.

In yet a further aspect of the invention there is provided a use of a compound of formula (I) or a pharmaceutically acceptable salt or prodrug thereof in the manufacture of a medicament for administration with a glucocorticoid for the treatment or prophylaxis of a disease or condition for which treatment of a glucocorticoid is indicated.

In yet a further aspect of the invention there is provided a use of a glucocorticoid and a compound of formula (I) or a pharmaceutically acceptable salt or prodrug thereof in the manufacture of a medicament for the treatment or prophylaxis of a disease or condition for which treatment with a glucocorticoid is indicated.

In preferred embodiments, the compounds of formula (I) or a pharmaceutically acceptable salt or prodrug thereof are used to treat or prevent a disease or condition, particularly in a human subject.

In yet a further aspect of the invention, there is provided a compound of formula (II) or a pharmaceutically acceptable salt or prodrug thereof:

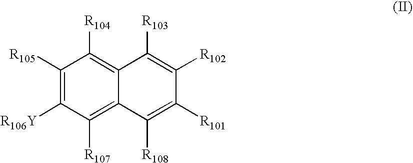

Wherein Y is selected from —O—, —NH—, —NC₁₋₃alkyl or —S(O)_{q}—

R₁₀₁ is selected hydrogen, C₁₋₆alkyl, CO₂H or CO₂C₁₋₆alkyl;

R₁₀₂ is selected from C₁₋₂₀alkyl, C₂₋₂₀alkenyl, CO₂H, CO₂C₁₋₂₀alkyl, CO₂C₂₋₂₀alkenyl, CO₂(CH₂)ₘR₁₀₉, SO₃H, SO₃C₁₋₂₀alkyl, SO₃C₂₋₃₀alkenyl, SO₃(CH₂)ₘR₁₀₉, C(O)C₁₋₂₀alkyl or (CH₂)ₘR₁₁₀;

R₁₀₃ is selected from hydrogen, hydroxy or C₁₋₃alkyl;

R₁₀₄ is selected from hydrogen, C₁₋₃alkyl, NH₂, NH(C₁₋₃alkyl), N(C₁₋₃alkyl)₂ or (CH₂)ₙOH;
R₁₀₅ is selected from hydrogen, (CH₂)ₙOH or (CH₂)ₙOC₁₋₃alkyl;

R₁₀₆ is selected from hydrogen, C₁₋₃alkyl, C(O)NH₂, C(O)NH(C₁₋₃alkyl), C(O)N(C₁₋₃alkyl)₂, C(S)NH₂, C(S)NH(C₁₋₃alkyl) or C(S)N(C₁₋₃alkyl)₂;

R₁₀₇ is selected from hydrogen, hydroxy, halo, amino, nitro, cyano, SO₃H or CO₂H;

R₁₀₈ is selected from hydrogen or methyl;

R₁₀₉ is selected from halogen, hydroxy, C₁₋₃alkoxy, NH₂, NH(C₁₋₃alkyl), N(C₁₋₃alkyl)₂, CO₂H or CO₂C₁₋₃alkyl;

R₁₁₀ is selected from hydroxy, C₁₋₃alkyl, halo, CO₂H, CO₂C₁₋₃alkyl, CN, NH₂, NH(C₁₋₃alkyl) or N(C₁₋₃alkyl)₂;

n is 0 or an integer from 1 to 3;

m is 0 or an integer from 1 to 20; and

wherein an alkyl, alkenyl or alkyloxy, group may be optionally substituted one or more times.

### BRIEF DESCRIPTION OF THE FIGURES

FIG. 1 graphically depicts the effect of a 1M ratio equivalent of 6,7-dimethoxy-2-naphthanoic acid on MIF-induced proliferation of human dermal fibroblasts.

FIG. 2 graphically depicts the effect of a 1M ratio equivalent of 6-hydroxy-2-naphthalene-sulfonic acid (compound 24) on MIF-induced proliferation of human dermal fibroblasts.

FIG. 3 graphically depicts the effect of different doses of 6,7-dihydroxynaphthalene-3-sulfonic acid (compound 6) on IL-1 induced COX-2 expression.

FIG. 4 graphically depicts the effect of a combination of dexamethasone and 6,7-dihydroxynaphthalene-3-sulfonic acid (compound 6) on IL-1 induced COX-2 expression.

FIG. 5 graphically depicts the arthritis index in the rat adjuvant-induced arthritis model for 6,7-dimethoxy-2-naphthanoic acid (compound 4).

FIG. 6 graphically depicts the synovial fluid cell number in the rat adjuvant-induced arthritis model for 6,7-dimethoxy-2-naphthanoic acid (compound 4).

FIG. 7 graphically depicts the effect of 6,7-dihydroxynaphthalene-3-sulfonic acid (compound 6) on in vivo serum IL-1 production in a murine endotoxic shock model.

FIG. 8 graphically depicts the effect of 6,7-dihydroxynaphthalene-3-sulfonic acid (compound 6) on in vivo serum IL-6 production in a murine endotoxic shock model.

FIG. 9 graphically depicts the cytotoxicity effect of a number of compounds in formula (I) in vitro.

FIG. 10 graphically depicts the effect of compound 6 on antigen-specific activation of splenic T lymphocytes from mice pre-immunised against mBSA. Activation is measured using tritiated (³H)-thymidine incorporation, as a measure of antigen-induced T cell proliferation.

FIG. 11 graphically depicts the in vivo effects of compound 23 on murine antigen induced arthritis, an animal model of rheumatoid arthritis.

FIG. 12 graphically depicts the inhibitory effect of compound 6 on the proliferation of S112 human dermal fibroblast cells treated with recombinant human MIF.

FIG. 13 graphically depicts the results of a dose-response experiment with compound 6 on endotoxin-induced interleukin-1 release from murine peritoneal macrophages.

### DETAILED DESCRIPTION OF THE INVENTION

As used herein, the term “alkyl”, either used alone or in compound terms such as NHCalkyl, N(Calkyl)₂ etc, refers to monovalent straight, branched or, where appropriate, cyclic aliphatic radicals having from 1 to 3, 1 to 6, 1 to 10 or 1 to 20 carbon atoms as appropriate, ie methyl, ethyl, n-propyl, iso-propyl, cyclopropyl, n-butyl, sec-butyl, t-butyl and cyclobutyl, n-pentyl, 1-methylbutyl, 2-methylbutyl, 3-methylbutyl, cyclopentyl, n-hexyl, 1- 2- 3- or 4-methylpentyl, 1- 2- or 3-ethylbutyl, 1 or 2-propylpropyl or cyclohexyl.

An alkyl group may be optionally substituted one or more times by halo (eg chloro, fluoro or bromo), CN, NO₂, CO₂H, CO₂C₁₋₆alkyl, CONH₂, CONH(C₁₋₆alkyl), CONH(C₁₋₆alkyl)₂, OH, hydroxyalkyl, alkoxy, methyl, ethyl, propyl, butyl, methoxy, ethoxy, propoxy, butoxy, acyl, carboxyalkyl, acetyl, trifluoromethyl, benzyloxy, phenoxy, NH₂, NH(C₁₋₆alkyl) or NH(C₁₋₆alkyl)₂. A preferred optional substituent is a polar substituent. Preferred optional substituents are hydroxy, NH₂ and CO₂H. Examples of alkoxy include methoxy, ethoxy, n-propoxy, iso-propoxy, cyclopropoxy, and butoxy (n-, sec- t- and cyclo) pentoxy and hexyloxy. The “alkyl” portion of an alkoxy group may be substituted as described above.

As used herein, the term “alkenyl” refers to straight, branched or, where appropriate, cyclic carbon containing radicals having one or more double bonds between carbon atoms. Examples of such radicals include vinyl, alkyl, butenyl, or longer carbon chains such as those derived from palmitoleic, oleic, linoleic, linolenic or arachidonic acids. An alkenyl group may be optionally substituted one or more times by halo (eg chloro, fluoro or bromo), CN, NO₂, CO₂H, CO₂C₁₋₆alkyl, CONH₂, CONH(C₁₋₆alkyl), CON(C₁₋₆alkyl)₂, OH, hydroxyalkyl, alkoxy, methyl, ethyl, propyl, butyl, methoxy, ethoxy, propoxy, butoxy, acyl, carboxyalkyl, acetyl, trifluoromethyl, benzyloxy, phenoxy, NH₂, NH(C₁₋₆alkyl) or NH(C₁₋₆alkyl)₂. A preferred optional substituent is a polar substituent, such as OH, NH₂ or CO₂H.

As used herein, the term “alkynyl” refers to straight or branched carbon containing radicals having one or more triple bonds between carbon atoms. Examples of such radicals include propargyl, butynyl and hexynyl. An alkynyl group may be optionally substituted one or more times by halo (eg chloro, fluoro or bromo), CN, NO₂, CO₂H, CO₂C₁₋₄alkyl, CONH₂, CONH(C₁₋₆alkyl), CON(C₁₋₆alkyl)₂, OH, hydroxyalkyl, alkoxy, methyl, ethyl, propyl, butyl, methoxy, ethoxy, propoxy, butoxy, acyl, carboxyalkyl, acetyl, trifluoromethyl, benzyloxy, phenoxy, NH₂, NH(C₁₋₆alkyl) or NH(C₁₋₆alkyl)₂. A preferred optional substituent is a polar substituent, such as NH₂, OH and CO₂H.

Examples of suitable NH(alkyl) and N(alkyl)₂ include methylamino, ethylamino, n-propylamino, iso-propylamino, dimethylamino, diethylamino and di-isopropylamino.

The term “halogen” (or “halo”) refers to fluorine (fluoro), chlorine (chloro), bromine (bromo) or iodine (iodo).

As used herein, “the characterising group of an amino acid” refers to the substituent at C₂ of a naturally occurring or non-naturally occurring amino acid and which defines the amino acid. The amino acid may be in the L or D configuration. For example, methyl is the characterising group of alanine, phenylmethyl is the characterising group of phenylalanine, hydroxymethyl is the characterising group of serine, hydroxyethyl is the characterising group of homoserine and n-propyl is the characterising group of norvaline.

The term “sugar” refers to a pyranosyl or furanosyl moiety such as derived from glucose, galactose, mannose, allose, altrose, gulose, idose, talose, ribose, arabinose or xylose. Derivatives of such sugars include deoxy or aminopyranosyl or furanosyl sugar derivatives. Each sugar moiety is incorporated into a compound of formula (I) through a hydroxy group of the sugar.

An aryl group refers to a C₆-C₁₂ aromatic carbocycle, for example, phenyl or naphthyl. An aryl group, either alone or part of a phenoxy, benzyl or benzyloxy group may be optionally substituted one or more times by halo (eg, chloro, fluoro or bromo), CN, NO₂, CO₂H, CO₂C₁₋₆alkyl, CONH₂, CONH(C₁₋₆alkyl), CON(C₁₋₆alkyl)₂, OH, hydroxyalkyl, alkoxy, methyl, ethyl, propyl, butyl, methoxy, ethoxy, propoxy, butoxy, acyl, carboxyalkyl, acetyl, trifluoromethyl, benzyloxy, phenoxy, NH₂, NH(C₁₋₆alkyl) or NH(C₁₋₆alkyl)₂, particularly hydroxy, or hydroxyalkyl or halo.

As used herein, the term “heterocyclyl” refers to a cyclic, aliphatic or aromatic radical containing at least one heteroatom independently selected from O, N or S. Examples of suitable heterocyclyl groups include furyl, pyridinyl, pyrimidinyl, pyrazolyl, piperidinyl, pyrrolyl, thiophenyl, oxazolyl, imidazolyl, thiazolyl, isoxazolyl, isothiazolyl, quinolyl, isoquinolyl, indolyl, benzofuranyl, benzothiophenyl, triazolyl, tetrazolyl, oxadiazolyl and purinyl. A heterocyclyl group may be optionally substituted one or more times by halo (eg, chloro, fluoro or bromo), CN, NO₂, CO₂H, CO₂C₁₋₆alkyl, CONH₂, CONH(C₁₋₆alkyl), CON(C₁₋₆alkyl)₂, OH, hydroxyalkyl, alkoxy, methyl, ethyl, propyl, butyl, methoxy, ethoxy, propoxy, butoxy, acyl, carboxyalkyl, acetyl, trifluoromethyl, benzyloxy, phenoxy, NH₂, NH(C₁₋₆alkyl) or NH(C₁₋₆alkyl)₂.

In a first aspect, the present invention provides a method of inhibiting cytokine or biological activity of MIF comprising contacting MIF with a cytokine or biological activity inhibiting effective amount of a compound of formula (I), or a pharmaceutically acceptable salt or prodrug thereof

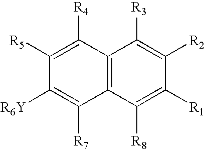

wherein

Y is O, NR₉ or S(O)_{q},

R₁ is selected from hydrogen, C₁₋₆alkyl, —(CR₁₀R_{10′})ₙhalo, —(CR₁₀R_{′})ₙOR₁₁, —(CR₁₀R_{10′})ₙ—SR₁₁, —(CR₁₀R_{10′})ₙ—N(R₁₂)₂, —(CR₁₀R_{10′})ₙS(O)R₁₁, —(CR₁₀R_{10′})ₙS(O)₂R₁₁, —(CR₁₀R_{10′})ₙ—S(O)₃R₁₁, —(CR₁₀R_{10′})ₙC(O)R₁₃, —(CR₁₀R_{10′})ₙ—C(═NR₁₄)R₁₅ or —(CR₁₀R_{10′})ₙR₁₆;

R₂ is selected from hydrogen, C₁₋₂₀alkyl, C₂₋₂₀alkenyl, C₂₋₂₀alkynyl, —(CR₁₀R_{10′})ₘOR₁₇, —(CR₁₀R_{10′})ₘSR₁₇, —(CR₁₀R_{10′})ₘNR₁₈R₁₉, —(CR₁₀R_{10′})ₘS(O)R₂₀, —(CR₁₀R_{10′})ₘS(O)₂R₂₀, —(CR₁₀R_{10′})ₘC(O)R₂₀, —(CR₁₀R_{10′})ₘC(S)R₂₀, —(CR₁₀R_{′})ₘC(═NR₁₁)R₁₅ or —(CR₁₀R_{10′})ₘR16;

R₃, R₄ and R₅ are independently selected from hydrogen, C₁₋₃alkyl, —(CR₁₀R_{10′})ₙN(R₁₄)₂, —(CR₁₀R_{10′})ₙOR₁₄, —(CR₁₀R_{10′})ₙ—SR₁₄ or —(CR₁₀R_{10′})ₙhalo;

R₆ is selected from hydrogen, C₁₋₆alkyl, —C(O)C₁₋₆alkyl, —C(O)N(R₉)₂—, —C(S)N(R₉)₂— or —(CR₁₀R_{10′})ₙR₂₁, or R₆Y and R₅ together may form —X—(CH₂)ₜ-Z-, where X and Z may be independently selected from O, S or NR₁₄;

R₇ and R₈ are independently selected from hydrogen, C₁₋₃alkyl, C₂₋₃alkenyl, C₂₋₃alkynyl or —(CR₁₀R_{10′})ₙR₂₂;

Each R₉ is independently selected from hydrogen or C₁₋₆alkyl;

Each R₁₀ and R_{10′}is independently selected from hydrogen, C₁₋₆alkyl, C₂₋₆alkenyl, C₂₋₆alkynyl, halogen, OR₁₁, SR₁₁, C₁₋₃alkoxy, CO₂R₁₄, N(R₁₄)₂, CN, NO₂, aryl or heterocyclyl;

R₁₁ is hydrogen or C₁₋₆alkyl;

Each R₁₂ is independently selected from hydrogen, C₁₋₆alkyl, C(═NR₁₄)R₁₅, NH—C(═NR₁₄)R₁₅, C(O)R₁₄ or C(S)R₁₄;

R₁₃ is hydrogen, C₁₋₆alkyl, OR₁₄, SR₁₄ or N(R₁₄)₂;

Each R₁₄ is independently selected from hydrogen or C₁₋₃alkyl;

R₁₅ is C₁₋₆alkyl, NH₂, NH(C₁₋₃alkyl) or N(C₁₋₃alkyl)₂, OR₂₃ or SR₂₃;

R₁₆ is hydroxy, C₁₋₃alkoxy, SH, SC₁₋₃alkyl, halo, C(O)R₃₁, C(R₂₄)₃, CN, aryl or heterocyclyl;

R₁₇ is selected from hydrogen, C₁₋₂₀alkyl, C₂₋₂₀alkenyl, C₂₋₂₀alkynyl, (CR₂₆R₂₆)ₛR₂₇, C(O)R₂₅, CO₂R₂₅, C(S)R₂₅, C(S)OR₂₅, S(O)R₂₅, S(O)₂R₂₅, [C(O)CH(R₂₉)NH]ᵣ—R₂₃ or [sugar]ᵣ;

R₁₈ and R₁₉ are independently selected from hydrogen, C₁₋₂₀alkyl, C₂₋₂₀alkenyl, C₂₋₂₀alkynyl, (CR₂₆R₂₆)ₛR₂₇, C(O)R₂₅, C(S)R₂₅, S(O)R₂₅, S(O)₂R₂₅, [C(O)CH(R₂₉)NH]ᵣ—R₂₃, [sugar]ᵣ, C(═NR₂₃)NH₂ or NH—C(═NR₂₃)NH₂;

R₂₀ is selected from hydrogen, C₁₋₂₀alkyl, C₂₋₂₀alkenyl, C₂₋₂₀alkynyl, OR₂₈, SR₂₈, N(R₂₈)₂, [NH—CHR₂₉C(O)]ᵣ—OR₂₃, [sugar]ᵣ or (CR₂₆R_{26′})ₛR₂₇;

R₂₁ is OR₂₈, SR₂₈, halo or N(R₂₅)₂;

R₂₂ is halo, CO₂H, SO₃H, NO₂, NH₂, CO₂C₁₋₃alkyl, SO₃C₁₋₃alkyl or C(R₂₄)₃;

R23 is hydrogen or C₁₋₃alkyl;

Each R₂₄ is independently selected from hydrogen, Cl or F;

Each R₂₅ is independently selected from hydrogen, C₁₋₂₀alkyl, C₂₋₂₀alkenyl, C₂₋₂₀alkynyl, aryl or (CR₂₆R₂₆)ₛR₂₇;

Each R₂₆ and R_{26′}is independently selected from hydrogen, C₁₋₆alkyl, C₂₋₆alkenyl, C₂₋₆alkynyl, halogen, hydroxy, C₁₋₃alkoxy, CO₂H, CO₂C₁₋₃alkyl, NH₂, NH(C₁₋₃alkyl), N(C₁₋₃alkyl)₂, CN, NO₂, aryl or heteroaryl;

R₂₇ is hydroxy, C₁₋₃alkoxy, SH, SC₁₋₃alkyl, halo, NH₂, NH(C₁₋₃alkyl), N(C₁₋₃alkyl)₂, C(O)R₃₁, aryl or heterocyclyl;

Each R₂₈ is independently selected from hydrogen, C₁₋₂₀alkyl, C₂₋₂₀alkenyl, C₂₋₂₀alkynyl or (CR₂₆R_{26′})R₃₀;

R₂₉ is the characterising group of an amino acid;

R₃₀ is halogen, hydroxy, C₁₋₃alkoxy, NH₂, NH(C₁₋₃alkyl), N(C₁₋₃alkyl)₂, C(O)R₃₁, aryl or heterocyclyl;

R₃₁ is C₁₋₃alkyl, OH, C₁₋₃alkoxy, aryl, aryloxy, heterocyclyl or heterocyclyloxy;,

q is 0, 1, 2 or 3;

n is 0, 1, 2 or 3;

m is 0 or 1 to 20;

r is 1 to 5;

s is 1 to 10; and

t is 1 or 2;

wherein an alkyl, alkenyl, alkynyl, alkyloxy, aryl or heterocyclyl group may be optionally substituted one or more times.

In a preferred embodiment, one or more of the following definitions apply:

Y is O, NH, NC₁₋₆alkyl, or S(O)_{q} wherein q is 0, 1, 2 or 3;

R₁ is hydrogen, C₁₋₆alkyl, (CH₂)ₙOH, (CH₂)ₙNH₂, (CH₂)ₙSH, (CH₂)ₙCF₃, (CH₂)ₙCO₂H, (CH₂)ₙCO₂C₁₋₃alkyl, (CH₂)ₙC(O)NH₂, (CH₂)ₙC(O)NHC₁₋₃alkyl, (CH₂)ₙC(O)N(C₁₋₃alkyl)₂, (CH₂)ₙSO₃H or (CH₂)ₙSO₃C₁₋₃alkyl, where n is 0, 1, 2 or 3; preferably H, CO₂H or CO₂C₁₋₃alkyl;

R₂ is selected from C₂₋₂₀alkyl, C₁₋₂₀alkenyl, (CR₁₀R_{10′})ₘOH, (CR₁₀R_{10′})ₘOC₁₋₂₀alkyl, (CR₁₀R_{10′})ₘOC₂₋₂₀alkenyl, (CR₁₀R_{10′})ₘOC(O)C₁₋₂₀alkyl, (CR₁₀R_{10′})ₘOC(O)C₂₋₂₀alkenyl, (CR₁₀R_{10′})ₘOC(O)aryl, (CR₁₀R_{10′})ₘO[C(O)CH(R₂₉)NH]ᵣ—H, (CR₁₀R_{10′})ₘO[sugar]ᵣ, (CR₁₀R_{10′})ₘNHC₁₋₂₀alkyl, (CR₁₀R_{10′})ₘN(C₁₋₂₀alkyl)₂, (CR₁₀R_{10′})ₘNHC₂₋₂₀alkenyl, (CR₁₀R_{10′})ₘN(C₂₋₂₀alkenyl)₂, (CR₁₀R_{10′})ₘN(C₁₋₂₀alkyl)(C₂₋₂₀alkenyl), (CR₁₀R_{10′})ₘNHC(O)C₁₋₂₀alkyl, (CR₁₀R_{10′})ₘNHC(O)C₂₋₂₀alkenyl, (CR₁₀R_{10′})ₘNHC(O)aryl, (CR₁₀R_{10′})ₘNH[C(O)CH(R₂₉)NH]ᵣ—H, (CR₁₀R_{10′})ₘNH-[sugar]ᵣ, (CR₁₀R_{10′})ₘSO₃H, (CR₁₀R_{10′})ₘSO₃C₁₋₂₀alkyl, (CR₁₀R_{10′})ₘSO₃C₂₋₂₀alkenyl, (CR₁₀R_{10′})ₘC(O)C₁₋₂₀alkyl, (CR₁₀R_{10′})ₘC(O)C₂₋₂₀alkenyl, (CR₁₀R_{10′})ₘCO₂H, (CR₁₀R_{10′})ₘCO₂C₁₋₂₀alkyl, (CR₁₀R_{10′})ₘCO₂₋₂₀alkenyl, (CR₁₀R_{10′})ₘC(O)NHC₁₋₂₀alkyl, (CR₁₀R_{10′})ₘC(O)N(C₁₋₂₀alkyl)₂, (CR₁₀R_{10′})ₘC(O)NHC₂₋₂₀alkenyl, (CR₁₀R_{10′})ₘC(O)N(C₂₋₂₀alkenyl)₂, (CR₁₀R_{10′})ₘC(O)N(C₁₋₂₀alkyl)(C₂₋₂₀alkenyl), (CR₁₀R_{10′})ₘC(O)[NHCH(R₂₉)C(O)]ᵣ—OH, (CR₁₀R_{10′})ₘC(O)[sugar]ᵣ, (CR₁₀R_{10′})ₘhalo, (CR₁₀R_{10′})ₘCN, (CR₁₀R_{10′})ₘheterocyclyl, (CR₁₀R_{10′})ₘaryl, (CR₁₀R_{10′})ₘNHC(═NH)NH₂, (CR₁₀R_{10′})ₘSO₂NHC₁₋₂₀alkyl, (CR₁₀R_{10′})ₘC(O)O(CH₂)₁₋₁₀CO₂H or (CR₁₀R_{10′})ₘC(O)O(CH₂)₁₋₁₀CO₂C₁₋₃alkyl; wherein each R₁₀ and R_{10′} is independently selected from hydrogen, C₁₋₆alkyl, C₂₋₆alkenyl,

C₂₋₆alkynyl, halogen, OH, OC₁₋₆alkyl, CO₂H, CO₂C₁₋₃alkyl, NH₂, NHC₁₋₃alkyl, —N(C₁₋₃alkyl)₂, CN, NO₂, aryl or heterocyclyl; R₂₉ is the characterising group of an amino acid, m is 0 or an integer from 1 to 20 and r is an integer from 1 to 5;

R₃ is selected from hydrogen, halo, NH₂, OH, OC₁₋₃alkyl, SH or SC₁₋₃alkyl, preferably hydrogen, OH or OC₁₋₃alkyl;

R₄ is selected from hydrogen, halogen, C₁₋₃alkyl, (CH₂)ₙNH₂, (CH₂)ₙNHC₁₋₃alkyl, (CH₂)ₙNH(C₁₋₃alkyl)₂, (CH₂)ₙOH or (CH₂)ₙOC₁₋₃alkyl, preferably hydrogen, C₁₋₃alkyl, (CH₂)ₙNH₂, (CH₂)ₙOH or (CH₂)ₙOC₁₋₃alkyl;

R₅ is selected from hydrogen, halogen, (CH₂)ₙNH₂, (CH₂)ₙOH, (CH₂)ₙOC₁₋₃alkyl, (CH₂)ₙSH or (CH₂)ₙSC₁₋₃alkyl; preferably hydrogen, (CH₂)ₙOH or (CH₂)ₙOC₁₋₃alkyl;

R₆ is selected from hydrogen, C₁₋₃alkyl, C(O)C₁₋₃alkyl, C(O)NH(C₁₋₃alkyl), C(O)N(C₁₋₃alkyl)₂, C(S)NH(C₁₋₃alkyl) or C(S)N(C₁₋₃alkyl)₂; or R₅ and R₆Y taken together form —X—(CH₂)ₜ-Z- wherein X and Z are independently selected from O and S and t is 1 or 2;

R₇ is selected from hydrogen, C₁₋₃alkyl, (CH₂)ₙSO₃H, (CH₂)ₙNO₂, (CH₂)ₙOH, (CH₂)ₙCO₂H, (CH₂)ₙNH₂, (CH₂)ₙhalo, (CH₂)CH₂halo, (CH₂)ₙCH(halo)₂ or (CH₂)ₙC(halo)₃, preferably hydrogen, (CH₂)ₙSO₃H, (CH₂)ₙNO₂, (CH₂)NH₂, or (CH₂)ₙhalo;

R₈ is selected from hydrogen, C₁₋₃alkyl, or (CH₂)ₙR₂₂, wherein R₂₂ is halo, CH₂halo, CH(halo)₂ or C(halo)₃ and n is 0, 1, 2 or 3; preferably hydrogen;

At least one of R₁₀ and R_{10′} in each (CR₁₀R_{10′}) is hydrogen and wherein the number of (CR₁₀R_{10′}) as designated by n is greater than 2, preferably less then 2 of R₁₀ and R_{10′} are other than hydrogen, and wherein the number of (CR₁₀R_{10′}) as designated by m is greater than 5, preferably less than 5 of R₁₀ and R_{10′} are other than hydrogen; preferably (CR₁₀R_{10′})ₙ and (CR₁₀R_{10′})ₘ represent an unsubstituted alkylene chain with n or m designating the number of methylene groups in the chain.

At least one of R₂₆ and R₂₆ is hydrogen in each (CR₂₆R_{26′}) and wherein the number of (CR₂₆R_{26′}) as designated by s is greater than 5, preferably less than 5 of R₂₆ and R_{26′} are other than hydrogen, more preferably (CR₂₆R_{26′})ₛ represents an unsubstituted alkylene chain with s designating the number of methylene groups in the chain.

In certain preferred forms of the invention, the compounds of formula (I) comprise:

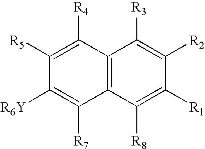

wherein

Y is O, NR₉ or S(O)_{q};

R₁ is hydrogen, C₁₋₆alkyl, —(CH₂)ₙC(O)R₁₃, —(CH₂)ₙS(O)₃R₁₁, —(CH₂)ₙNH₂, —(CH₂)ₙOH, —(CH₂)ₙSH or —(CH₂)ₙCF₃, where R₁₁ and R₁₃ are defined above;

R₂ is selected from hydrogen, C₁₋₂₀alkyl, C₂₋₂₀alkenyl, C₂₋₂₀alkynyl, —(CR₁₀R_{10′})ₘOR₁₇, —(CR₁₀R_{10′})ₘSR₁₇, —(CR₁₀R_{10′})ₘNR₁₈R₁₉, —(CR₁₀R_{10′})ₘS(O)R₂₀, —(CR₁₀R_{10′})ₘS(O)₂R₂₀, —(CR₁₀R_{10′})ₘC(O)R₂₀, —(CR₁₀R_{10′})ₘC(S)R₂₀, —(CR₁₀R_{10′})ₘC(C═NR₁₁)R₁₅ or —(CR₁₀R_{10′})ₘR₁₆, where m, R₁₀, R_{10′}, R₁₁, R₁₅, R₁₆, R₁₇, R₁₈, R₁₉, R₂₀ are as defined above;

R₃ is selected from hydrogen, halo, amino, OH, OC₁₋₃alkyl or SH;

R₄ is selected from hydrogen, halogen, C₁₋₃alkyl, (CH₂)ₙNH₂, (CH₂)ₙNHC₁₋₃alkyl, (CH₂)ₙNH(C₁₋₃alkyl)₂, (CH₂)ₙOH or (CH₂)ₙOC₁₋₃alkyl;

R₅ is selected from hydrogen, halogen, (CH₂)ₙNH₂, (CH₂)ₙOH, (CH₂)ₙOC₁₋₃alkyl, (CH₂)ₙSH or (CH₂)ₙSC₁₋₃alkyl;

R₆ is hydrogen, C₁₋₃alkyl, CH₂halo, C(O)NH(C₁₋₃alkyl), C(O)N(C₁₋₃alkyl)₂, C(S)NH(C₁₋₃alkyl), C(S)N(C₁₋₃alkyl)₂, CH₂OH or CH₂SH;

or R₅ and YR₆ together form X—(CH₂)ₜ-Z wherein X and Z are independently selected from O and S;

R₇ is selected from hydrogen, C₁₋₃alkyl, or (CH₂)ₙSO₃H, (CH₂)ₙNO₂, (CH₂)ₙOH, (CH₂)ₙCO₂H, (CH₂)ₙNH₂, (CH₂)ₙhalo, (CH₂)ₙCH₂halo, (CH₂)ₙCH(halo)₂ or (CH₂)ₙC(halo)₃,

R₈ is hydrogen, C₁₋₃alkyl or (CH₂)ₙhalo, and

q and n are 0, 1, 2 or 3.

More preferably, the compounds of formula (I) comprise:

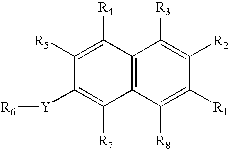

wherein

Y is O, NR₉ or S(O)_{q};

R₁ is hydrogen, (CH₂)ₙCO₂H, (CH₂)ₙCO₂C₁₋₃alkyl, (CH₂)ₙSO₃H, (CH₂)ₙNH₂, C₁₋₃alkyl (CH₂)ₙOH or (CH₂)ₙCF₃;

R₂ is selected from hydrogen, C₁₋₂₀alkyl, C₂₋₂₀alkenyl, C₂₋₂₀alkynyl, —(CR₁₀R_{10′})ₘOR₁₇, —(CR₁₀R_{10′})ₙSR₁₇, —(CR₁₀R_{10′})ₘNR₁₈R₁₉, —(CR₁₀R_{10′})ₘS(O)R₂₀, —(CR₁₀R_{10′})ₘS(O)₂R₂₀, —(CR₁₀R_{10′})ₘC(O)R₂₀, —(CR₁₀R_{10′})ₘC(S)R₂₀, —(CR₁₀R_{10′})ₘC(═NR₁₁)R₁₅ or —(CR₁₀R_{10′})ₘR₁₆, where m, R₁₀, R_{10′}, R₁₁, R₁₅, R₁₆, R₁₇, R₁₈, R₁₉, R₂₀ are as defined above;

R₃ is selected from hydrogen, OH or OC₁₋₃alkyl,

R₄ is selected from hydrogen, C₁₋₃alkyl, (CH₂)ₙNH₂, (CH₂)ₙOH or (CH₂)ₙOC₁₋₃alkyl;

R₅ is hydrogen, (CH₂)ₙOH or (CH₂)ₙOC₁₋₃alkyl;

R₆ is hydrogen, C₁₋₃alkyl, CH₂halo, C(O)NH(C₁₋₃alkyl), C(O)N(C₁₋₃alkyl)₂, C(S)NH(C₁₋₃alkyl), C(S)N(C₁₋₃alkyl)₂, CH₂OH or CH₂SH;

or R₅ and R₆Y are taken together to form —O—(CH₂)ₜ—O where t is 1 or 2;

R₇ is selected from hydrogen, (CH₂)ₙSO₃H, (CH₂)ₙNO₂, (CH₂)ₙNH₂, or (CH₂)ₙhalo

R₈ is hydrogen, CH₃, CF₃ or CCl₃;

and q and n are 0, 1, 2 or 3.

More preferably, the compounds of formula (I) comprise:

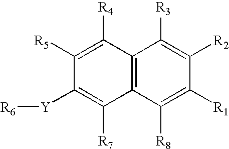

wherein

Y is O, NR₉ or S(O)_{q};

R₁ is hydrogen, (CH₂)ₙCO₂H, (CH₂)ₙCO₂C₁₋₃alkyl, (CH₂)ₙSO₃H, (CH₂)ₙNH₂, C₁₋₃alkyl, (CH₂)ₙOH or (CH₂)ₙCF₃;

R₂ is selected from hydrogen, C₁₋₂₀alkyl, C₂₋₂₀alkenyl, —(CR₁₀R_{10′})ₘOH, —(CR₁₀R_{10′})ₘNHC₁₋₂₀alkyl, —(CR₁₀R_{10′})ₘNH[C(O)CH(R₂₉)NH]—H, —(CR₁₀R_{10′})ₘSO₃H, —(CR₁₀R_{10′})ₘSO₃C₁₋₂₀alkyl, —(CR₁₀R_{10′})ₘC(O)C₁₋₂₀alkyl, —(CR₁₀R_{10′})ₘCO₂H, —(CR₁₀R_{′})ₘCO₂C₁₋₂₀alkyl, —(CR₁₀R_{10′})ₘCN, —(CR₁₀R_{10′})ₘhalo, —CR₁₀R_{10′})ₘaryl, —(CR₁₀R_{10′})ₘheterocyclyl, —(CR₁₀R_{10′})ₘNHC(═NH)NH₂, —(CR₁₀R_{10′})ₘSO₂NHC₁₋₂₀alkyl, CO₂(CH₂)₁₋₁₀CO₂H or CO₂(CH₂)₁₋₁₀CO₂C₁₋₃alkyl, where m, R₁₀ and R_{10′} are as defined above;

R₃ is selected from hydrogen, OH or OC₁₋₃alkyl,

R₄ is selected from hydrogen, C₁₋₃alkyl, (CH₂)ₙNH₂, (CH₂)ₙOH or (CH₂)ₙOC₁₋₃alkyl;

R₅ is hydrogen, (CH₂)ₙOH or (CH₂)ₙOC₁₋₃alkyl;

R₆ is hydrogen, C₁₋₃alkyl, CH₂halo, C(O)NH(C₁₋₃alkyl), C(O)N(C₁₋₃alkyl)₂, C(S)NH(C₁₃alkyl) or C(S)N(C₁₋₃alkyl)₂, CH₂OH or CH₂SH;

or R₅ and R₆ are taken together to form —O—(CH₂)ₜ—O where t is 1 or 2;

R₇ is selected from hydrogen, (CH₂)ₙSO₃H, (CH₂)ₙNO₂, (CH₂)ₙNH₂, or (CH₂)ₙhalo;

R₈ is hydrogen, CH₃, CF₃ or CCl₃;

and q and n are 0, 1, 2 or 3.

Yet further preferred compounds of formula (I) are those of formula (II) or a pharmaceutically acceptable salts or prodrugs thereof:

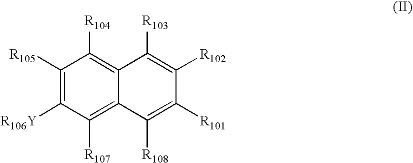

wherein Y is selected from —O—, —NH—, —NC₁₋₃alkyl- or —S(O)_{q}—;

R₁₀₁ is selected hydrogen, C₁₋₆alkyl, CO₂H or CO₂C₁₋₆alkyl;

R₁₀₂ is selected from C₁₋₂₀alkyl, C₂₋₂₀alkenyl, CO₂H, CO₂C₁₋₂₀alkyl, CO₂C₂₋₂₀alkenyl, CO₂(CH₂)ₘR₁₀₉, SO₃H, SO₃C₁₋₂₀alkyl, SO₃C₂₋₂₀alkenyl, SO₃(CH₂)ₘR₁₀₉, C(O)C₁₋₂₀alkyl or (CH₂)ₘR₁₁₀;

R₁₀₃ is selected from hydrogen, hydroxy, methoxy or C₁₋₃alkyl;

R104 is selected from hydrogen, C₁₋₃alkyl, NH₂, NH(C₁₋₃alkyl), N(C₁₋₃alkyl)₂ or (CH₂)ₙOH;

R₁₀₅ is selected from hydrogen, (CH₂)ₙOH or (CH₂)ₙOC₁₋₃alkyl;

R₁₀₆ is selected from hydrogen, C₁₋₃alkyl, C(O)NH₂, C(O)NH(C₁₋₃alkyl), C(O)N(C₁₋₃alkyl)₂, C(S)NH₂, C(S)NH(C₁₋₃alkyl) or C(S)N(C₁₋₃alkyl)₂;

R₁₀₇ is selected from hydrogen, hydroxy, halo, amino, nitro, cyano, SO₃H or CO₂H;

R₁₀₈ is selected from hydrogen or methyl;

R₁₀₉ is selected from halogen, hydroxy, C₁₋₃alkoxy, NH₂, NH(C₁₋₃alkyl), N(C₁₋₃alkyl)₂, CO₂H or CO₂C₁₋₃alkyl;

R₁₁₀ is selected from hydroxy, C₁₋₃alkyl, halo, CO₂H, CO₂C₁₋₃alkyl, CN, NH₂, NH(C₁₋₃alkyl) or N(C₁₋₃alkyl)₂;

n is 0 or an integer from 1 to 3;

m is 0 or an integer from 1 to 20; and

wherein an alkyl, alkenyl or alkyloxy, group may be optionally substituted one or more times.

Examples of suitable compounds for use in the invention may include:

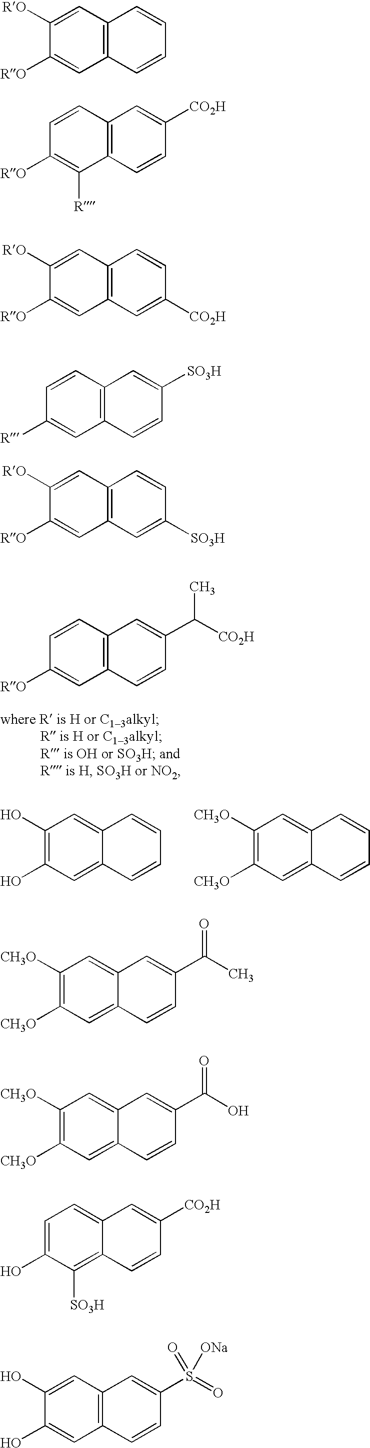

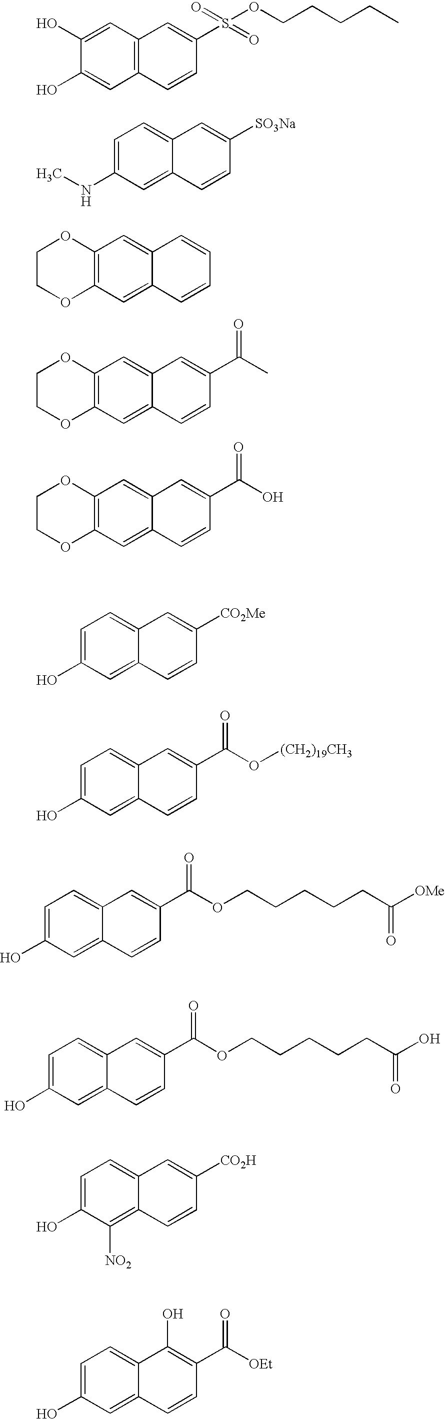

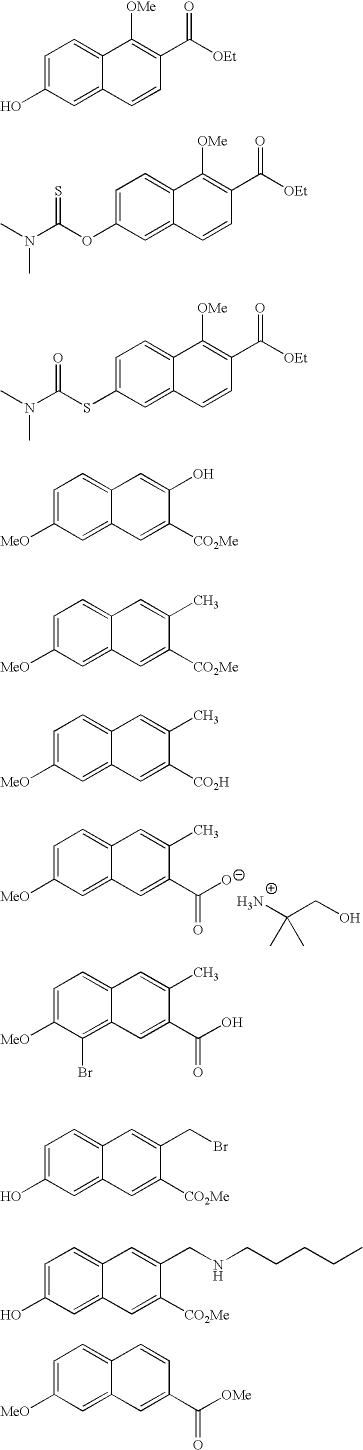

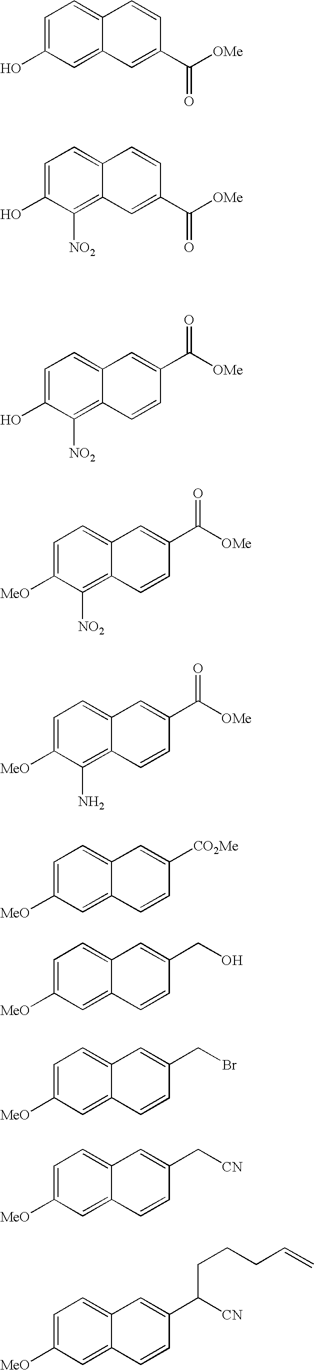

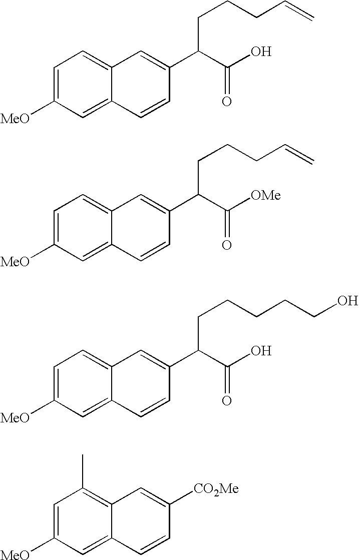

Compounds of formula (I) may be prepared using the methods depicted or described herein or known in the art for example (12). It will be understood that minor modifications to methods described herein or known in the art may be required to synthesize particular compounds of formula (I). General synthetic procedures applicable to the synthesis of compounds may be found in standard references such as Comprehensive Organic Transformations, R. C. Larock, 1989, VCH Publishers and Advanced Organic Chemistry, J. March, 4th Edition (1992), Wiley InterScience, and references therein, and may include Friedel Crafts acylation and/or electrophilic aromatic substitution of the naphthalene nucleus followed, where appropriate, by synthetic conversion (using standard procedures) to the desired groups. It will also be recognised that certain reactive groups may require protection and deprotection during the synthetic process. Suitable protecting and deprotecting methods for reactive functional groups are known in the art for example in Protective Groups in Organic Synthesis, T. W. Green & P. Wutz, John Wiley & Son, 3^{rd} Edition, 1999. Thus, for certain embodiments of the invention, compounds of formula (I), where R₁ or R₂ is CO₂H, can be prepared in accordance with the exemplified general methods or steps depicted in any of Schemes 1-3. Suitable starting materials can be obtained commercially or prepared using methods known in the art. Methodology relating to Schemes 1 and 2 can be found in (13) and (14) respectively. Methods for derivatizing NH₂, SH and OH to provide further compounds of formula I are known in the art.

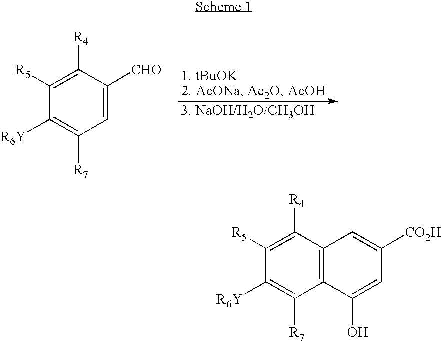

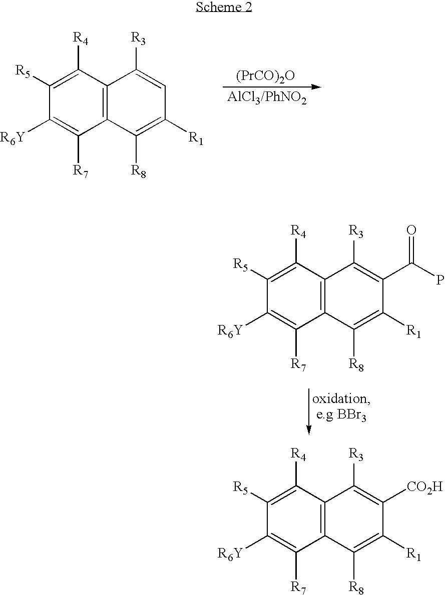

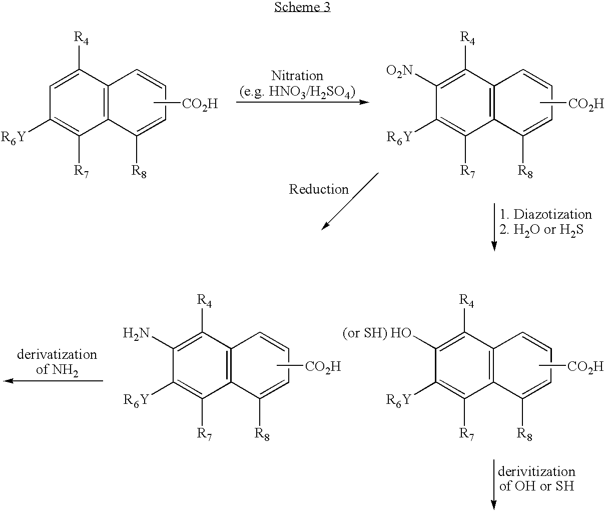

Conversion of a CO₂H group to the amide (CONH₂) can be carried out using standard procedures in the art. Conversion of the amide to C═NH(NH₂) can be achieved by aminolysis eg NH₃/dry methanol.

A methylene group can be inserted between the naphthalene nucleus and the carboxylic acid group by Amdt-Eistert synthesis, eg by conversion of the carboxylic acid to an acyl halide and conversion to the diazoketone. Rearrangement of the diazoketone (eg with silver oxide and water) affords access to the CH₂—CO₂H group. Repeating these steps allows for further incorporation of methylene groups. The CO₂H group can be converted as above.

In other embodiments, compounds of formula (I), where R₁ or R₂ is a substituted methyl group, can be prepared by conversion of R₁ or R₂ being a methyl substituent into a halomethyl substituent (eg by treatment with a N-halosuccinimide such as NBS) followed by nucleophilic substitution by an appropriate nucleophile and/or insertion of additional methylene groups by, for example, Wittig reaction (see Scheme 4 where R* can be (CH₂)ₘOH, (CH₂)ₘSH, (CH₂)ₘNH₂ (CH₂)ₘC(O)C₁₋₆alkyl, (CH₂)ₘOC(O)C₁₋₆alkyl, (CH₂)ₘOC₁₋₆alkyl, (CH₂)ₘOphenyl, (CH₂)ₘObenzyl, (CH₂)ₘNHC₁₋₆alkyl, (CH₂)ₘN(C₁₋₆alkyl)₂, (CH₂)ₘNHphenyl, (CH₂)ₘNHbenzyl, (CH₂)ₘSC₁₋₆alkyl, (CH₂)ₘSC(O)C₁₋₆alkyl, (CH₂)ₘSphenyl, (CH₂)ₘSbenzyl, (CH₂)ₘNHsugar, (CH₂)ₘSsugar, (CH₂)ₘOsugar, (CH₂)ₘNHC(O)C₁₋₆alkyl, (CH₂)ₘNHC(O)phenyl, (CH₂)ₘNHC(O)benzyl, (CH₂)ₘNHCO₂C₁₋₆alkyl, (CH₂)ₘNHCO₂phenyl, or (CH₂)ₘNHCO₂benzyl, where m is 0 or 1 to 20).

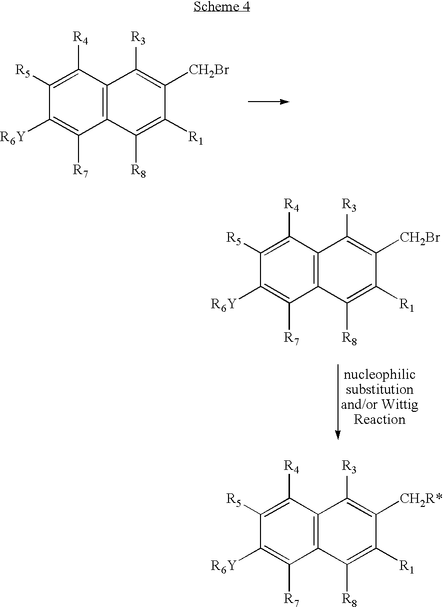

In other embodiments, compounds where an O, S or N atom is directly bonded to the naphthalene nucleus can be prepared by suitable substitution (derivatization) of the corresponding OH, SH or NH₂ group on the naphthalene nucleus eg by standard alkylating or acylating methodology.

In other embodiments, compounds where R₁ or R₂ is CH₂halo can be prepared by reaction of a suitable naphthalene carboxylic acid derivative with a reducing agent such as LiAlH₄, followed by halogenation, eg treatment with thionyl chloride.

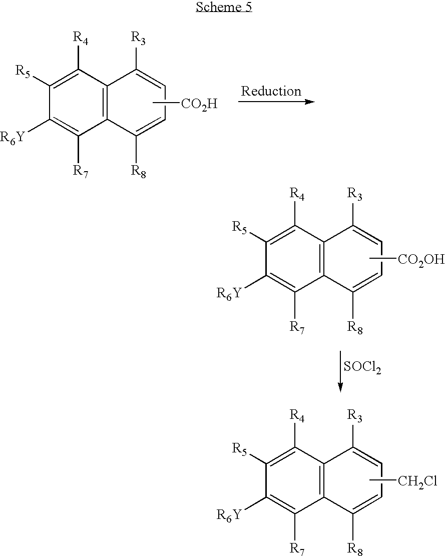

Coupling of compounds wherein R₁ or R₂ is CH₂halo with a C₁₋₆alkylhalide, halo(CH₂)_{n/m}heterocyclyl in the presence of CuLi affords the corresponding compounds where the R₁ and/or R₂ substituent is C₁₋₆alkyl, (CH₂)_{n/m}heterocyclyl.

Reaction of CH₂halo with NH₂—NH—C(═NH)—NH₂ in the presence of base affords access to compounds wherein R₁/R₂ is CH₂—NH—NH—C(═NH)—NH₂. Alternatively, reaction of the CH₂halo group with halo(CH₂)ₙNH—NH—C(═NH)—NH₂ (where n is 1 or 2), affords the group (CH₂)ₙNH—NH—C(═NH)—NH₂ where n is 2 or 3.

The term “salt, or prodrug” includes any pharmaceutically acceptable salt, ester, solvate, hydrate or any other compound which, upon administration to the recipient is capable of providing (directly or indirectly) a compound of formula (I) as described herein. The term “pro-drug” is used in its broadest sense and encompasses those derivatives that are converted in vivo to the compounds of the invention. Such derivatives would readily occur to those skilled in the art, and include, for example, compounds where a free hydroxy group is converted into an ester, such as an acetate, or where a free amino group is converted into an amide. Procedures for acylating hydroxy or amino groups of the compounds of the invention are well known in the art and may include treatment of the compound with an appropriate carboxylic acid, anhydride or acylchloride in the presence of a suitable catalyst or base.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will also be recognised that some compounds of formula (I) may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

In another aspect, the invention provides a method of treating, preventing or diagnosing a disease or condition wherein MIF cytokine or biological activity is implicated comprising the administration of a treatment, prevention or diagnostic effective amount of a compound of formula (I) or a pharmaceutically acceptable salt or prodrug thereof to a subject in need thereof.

In a further aspect, there is provided the use of a compound of formula (I) or a pharmaceutically acceptable salt or prodrug thereof in the manufacture of a medicament for the treatment, prevention or diagnosis of a disease or condition wherein MIF cytokine or biological activity is implicated.

In yet a further aspect, there is provided an agent for the treatment, prevention or diagnosis of a disease or condition where MIF cytokine or biological activity is implicated comprising a compound of formula (I) or a pharmaceutically acceptable salt or prodrug thereof.

As used herein, MIF includes human or other animal MIF and derivatives and naturally occurring variants thereof which at least partially retain MIF cytokine or biological activity. Thus, the subject to be treated may be human or other animal such as a mammal. Non-human subjects include, but are not limited to primates, livestock animals (eg sheep, cows, horses, pigs, goats), domestic animals (eg dogs, cats), birds and laboratory test animals (eg mice rats, guinea pigs, rabbits). MIF is also expressed in plants (thus “MIF” may also refer to plant MIF) and where appropriate, compounds of formula (I) may be used in botanical/agricultural applications such as crop control.

Reference herein to “cytokine or biological activity” of MIF includes the cytokine or biological effect on cellular function via autocrine, endocrine, paracrine, cytokine, hormone or growth factor activity, or via intracellular effects.

In particular, the invention provides a method of treating, diagnosing or preventing autoimmune diseases, solid or haemopoeitic tumours, or chronic or acute inflammatory diseases, including a disease or condition selected from the group comprising:
Rheumatic diseases (including but not limited to rheumatoid arthritis, osteoarthritis, psoriatic arthritis, polymyalgia rheumatica) spondyloarthropathies (including but not limited to ankylosing spondylitis, reactive arthritis, Reiter's syndrome), crystal arthropathies (including but not limited to gout, pseudogout, calcium pyrophosphate deposition disease), Lyme disease, connective tissue diseases (including but not limited to systemic lupus erythematosus, systemic sclerosis, polymyositis, dermatomyositis, Sjögren's syndrome), vasculitides (including but not limited to polyarteritis nodosa, Wegener's granulomatosis, Churg-Strauss syndrome), glomerulonephritis, interstitial nephritis, inflammatory bowel disease (including but not limited to ulcerative colitis, Crohn's disease), peptic ulceration, gastritis, oesophagitis, liver disease (including but not limited to cirrhosis, hepatitis), autoimmune diseases (including but not limited to diabetes mellitus, thyroiditis, myasthenia gravis, sclerosing cholangitis, primary biliary cirrhosis), pulmonary diseases (including but not limited to diffuse interstitial lung diseases, pneumoconioses, fibrosing alveolitis, asthma, bronchitis, bronchiectasis, chronic obstructive pulmonary disease, adult respiratory distress syndrome), cancers whether primary or metastatic (including but not limited to colon cancer, lymphoma, lung cancer, melanoma, prostate cancer, breast cancer, stomach cancer, leukemia, cervical cancer, multiple myeloma and metastatic cancer), atherosclerosis (eg ischaemic heart disease, myocardial infarction, stroke, peripheral vascular disease), disorders of the hypothalamic-pituitary-adrenal axis, brain disorders (eg dementia, Alzheimer's disease, multiple sclerosis, demyelinating diseases), corneal disease, iritis, iridocyclitis, cataracts, uveitis, sarcoidosis, diseases characterised by modified angiogenesis (eg diabetic retinopathy, rheumatoid arthritis, cancer), endometrial function (menstruation, implantation, parturition, endometriosis), psoriasis, endotoxic (septic) shock, exotoxic (septic) shock, infective (true septic) shock, other complications of infection, pelvic inflammatory disease, transplant rejection, allergies, allergic rhinitis, bone diseases (eg osteoporosis, Paget's disease), atopic dermatitis, UV(B)-induced dermal cell activation (eg sunburn, skin cancer), malarial complications, diabetes mellitus, pain, inflammatory consequences of trauma or ischaemia, testicular dysfunctions and wound healing,
   comprising the administration of a treatment, diagnosis or prevention effective amount of a compound of formula (I) or a pharmaceutically acceptable salt or prodrug thereof to a subject in need thereof.

In a preferred embodiment, the invention provides a method of treating, diagnosing or preventing autoimmune diseases, solid or haemopoeitic tumours, or chronic or acute inflammatory diseases, including a disease or condition selected from the group comprising rheumatic diseases (including but not limited to rheumatoid arthritis, osteoarthritis, psoriatic arthritis, polymyalgia rheumatica) spondyloarthropathies (including but not limited to ankylosing spondylitis, reactive arthritis, Reiter's syndrome), crystal arthropathies (including but not limited to gout, pseudogout, calcium pyrophosphate deposition disease), connective tissue diseases (including but not limited to systemic lupus erythematosus, systemic sclerosis, polymyositis, dermatomyositis, Sjögren's syndrome), glomerulonephritis, interstitial nephritis, inflammatory bowel disease (including but not limited to ulcerative colitis, Crohn's disease), peptic ulceration, gastritis, oesophagitis, liver disease (including but not limited to cirrhosis, hepatitis), autoimmune diseases (including but not limited to diabetes mellitus, thyroiditis, myasthenia gravis), pulmonary diseases (including but not limited to diffuse interstitial lung diseases, asthma, bronchitis, chronic obstructive pulmonary disease, adult respiratory distress syndrome), cancers whether primary or metastatic (including but not limited to colon cancer, lymphoma, lung cancer, melanoma, prostate cancer, breast cancer, stomach cancer, leukemia, cervical cancer, multiple myeloma and metastatic cancer), atherosclerosis (eg ischaemic heart disease, myocardial infarction), brain disorders (eg multiple sclerosis, demyelinating diseases), iritis, iridocyclitis, uveitis, sarcoidosis, diseases characterised by modified angiogenesis (eg diabetic retinopathy, rheumatoid arthritis, cancer), psoriasis, endotoxic (septic) shock, exotoxic (septic) shock, infective (true septic) shock, pelvic inflammatory disease, transplant rejection, allergies, allergic rhimtis, bone diseases (including but not limited to osteoporosis, Paget's disease), atopic dermatitis, malarial complications, diabetes mellitus, pain, inflammatory consequences of trauma or ischaemia, and wound healing, comprising the administration of a treatment, diagnosis or prevention effective amount of a compound of formula (I) or a pharmaceutically acceptable salt or prodrug thereof to a subject in need thereof.

In yet another preferred embodiment of the invention there is provided a method of treating, diagnosing or preventing autoimmune diseases, solid or haemopoeitic tumours, or chronic or acute inflammatory diseases, including a disease or condition selected from the group comprising rheumatic diseases (including but not limited to rheumatoid arthritis, osteoarthritis, psoriatic arthritis, polymyalgia rheumatica) spondyloarthropathies (including but not limited to ankylosing spondylitis, reactive arthritis), crystal arthropathies (including but not limited to gout, pseudogout, calcium pyrophosphate deposition disease), connective tissue diseases (including but not limited to systemic lupus erythematosus, systemic sclerosis, polymyositis, dermatomyositis), glomerulonephritis, interstitial nephritis, inflammatory bowel disease (including but not limited to ulcerative colitis, Crohn's disease), liver disease (including but not limited to cirrhosis, hepatitis), autoimmune diseases (including but not limited to diabetes mellitus, thyroiditis, myasthenia gravis,), pulmonary diseases (including but not limited to asthma, chronic obstructive pulmonary disease, adult respiratory distress 'syndrome), cancers whether primary or metastatic (including but not limited to colon cancer, lymphoma, lung cancer, leukemia, cervical cancer, multiple myeloma and metastatic cancer), atherosclerosis (eg ischaemic heart disease, myocardial infarction), brain disorders (eg multiple sclerosis, demyelinating diseases), uveitis, sarcoidosis, diseases characterised by modified angiogenesis (eg diabetic retinopathy, rheumatoid arthritis, cancer), psoriasis, endotoxic (septic) shock, exotoxic (septic) shock, infective (true septic) shock, transplant rejection, allergies, allergic rhinitis, bone diseases (including but not limited to osteoporosis, Paget's disease), atopic dermatitis, malarial complications, diabetes mellitus, pain, inflammatory consequences of trauma or ischaemia, and wound healing, comprising the administration of a treatment, diagnosis or prevention effective amount of a compound of formula (I) or a pharmaceutically acceptable salt or prodrug thereof to a subject in need thereof.

In yet another preferred embodiment, the invention provides a method of treating, diagnosing or preventing autoimmune diseases, solid or haemopoeitic tumours, or chronic or acute inflammatory diseases, including a disease or condition selected from the group comprising rheumatic diseases (including but not limited to rheumatoid arthritis, osteoarthritis, psoriatic arthritis, polymyalgia rheumatica) spondyloarthropathies (including but not limited to ankylosing spondylitis, reactive arthritis), connective tissue diseases (including but not limited to systemic lupus erythematosus, systemic sclerosis,), glomerulonephritis, interstitial nephritis, inflammatory bowel disease (including but not limited to ulcerative colitis, Crohn's disease), liver disease (including but not limited to cirrhosis, hepatitis), autoimmune diseases (including but not limited to diabetes mellitus, thyroiditis, myasthenia gravis,), pulmonary diseases (including but not limited to asthma, chronic obstructive pulmonary disease, adult respiratory distress syndrome), cancers whether primary or metastatic (including but not limited to colon cancer, lymphoma, lung cancer, leukemia, cervical cancer, multiple myeloma and metastatic cancer), atherosclerosis (eg ischaemic heart disease, myocardial infarction), brain disorders (eg multiple sclerosis, demyelinating diseases), psoriasis, transplant rejection, allergies, allergic rhinitis, atopic dermatitis, and wound healing, comprising the administration of a treatment, diagnosis or prevention effective amount of a compound of formula (I) or a pharmaceutically acceptable salt or prodrug thereof to a subject in need thereof.

In a further preferred embodiment, the invention provides a method of treating, diagnosing or preventing autoimmune diseases, or chronic or acute inflammatory diseases, including a disease or condition selected from the group comprising rheumatic diseases (including but not limited to rheumatoid arthritis, osteoarthritis, psoriatic arthritis, polymyalgia rheumatica) spondyloarthropathies (including but not limited to ankylosing spondylitis, reactive arthritis,), connective tissue diseases (including but not limited to systemic lupus erythematosus, systemic sclerosis,), glomerulonephritis, interstitial nephritis, inflammatory bowel disease (including but not limited toulcerative colitis, Crohn's disease), liver disease (including but not limited to cirrhosis, hepatitis), autoimmune diseases (including but not limited to diabetes mellitus, thyroiditis, myasthenia gravis,), pulmonary diseases (including but not limited to asthma, chronic obstructive pulmonary disease, adult respiratory distress syndrome), atherosclerosis (eg ischaemic heart disease, myocardial infarction), brain disorders (eg multiple sclerosis, demyelinating diseases), psoriasis, transplant rejection, allergies, allergic rhinitis, atopic dermatitis, and wound healing, comprising the administration of a treatment, diagnosis or prevention effective amount of a compound of formula (I) or a pharmaceutically acceptable salt or prodrug thereof to a subject in need thereof.

In yet a further preferred embodiment, the invention provides a method of treating, diagnosing or preventing autoimmune diseases, or chronic or acute inflammatory diseases, including a disease or condition selected from the group comprising rheumatic diseases (including but not limited to rheumatoid arthritis, psoriatic arthritis, polymyalgia rheumatica), spondyloarthropathies (including but not limited to ankylosing spondylitis,), connective tissue diseases (including but not limited to systemic lupus erythematosus), glomerulonephritis, interstitial nephritis, inflammatory bowel disease (including but not limited to ulcerative colitis, Crohn's disease), liver disease (including but not limited to cirrhosis, hepatitis), autoimmune diseases (including but not limited to diabetes mellitus, thyroiditis, myasthenia gravis,), pulmonary diseases (including but not limited to asthma, chronic obstructive pulmonary disease, adult respiratory distress syndrome), atherosclerosis (eg ischaemic heart disease, myocardial infarction), brain disorders (eg multiple sclerosis, demyelinating diseases), psoriasis, transplant rejection, allergic rhinitis, and atopic dermatitis, comprising the administration of a treatment, diagnosis or prevention effective amount of a compound of formula (I) or a pharmaceutically acceptable salt or prodrug thereof to a subject in need thereof.

In yet a further preferred embodiment, the invention provides a method of treating, diagnosing or preventing autoimmune diseases, or chronic or acute inflammatory diseases, including a disease or condition selected from the group comprising rheumatic diseases (including but not limited to rheumatoid arthritis, psoriatic arthritis, polymyalgia rheumatica), spondyloarthropathies (including but not limited to ankylosing spondylitis), connective tissue diseases (including but not limited to systemic lupus erythematosus), glomerulonephritis, inflammatory bowel disease (including but not limited to ulcerative colitis, Crohn's disease), pulmonary diseases (including but not limited to asthma, chronic obstructive pulmonary disease, adult respiratory distress syndrome), atherosclerosis (eg ischaemic heart disease, myocardial infarction), brain disorders (eg multiple sclerosis, demyelinating diseases), psoriasis, and transplant rejection, comprising the administration of a treatment, diagnosis or prevention effective amount of a compound of formula (I) or a pharmaceutically acceptable salt or prodrug thereof to a subject in need thereof.

A further aspect of the invention provides for the use of a compound of formula (I) or a pharmaceutically acceptable salt or prodrug thereof in the manufacture of a medicament for the treatment of a disease or condition as above.

As used herein, the term “effective amount” relates to an amount of compound which, when administered according to a desired dosing regimen, provides the desired MIF cytokine inhibiting or treatment or therapeutic activity, or disease/condition prevention. Dosing may occur at intervals of minutes, hours, days, weeks, months or years or continuously over any one of these periods. A cytokine or biological activity inhibiting amount is an amount which will at least partially inhibit the cytokine or biological activity of MIF. A therapeutic, or treatment, effective amount is an amount of the compound which, when administered according to a desired dosing regimen, is sufficient to at least partially attain the desired therapeutic effect, or delay the onset of, or inhibit the progression of or halt or partially or fully reverse the onset or progression of a particular disease condition being treated. A prevention effective amount is an amount of compound which when administered according to the desired dosing regimen is sufficient to at least partially prevent or delay the onset of a particular disease or condition. A diagnostic effective amount of compound is an amount sufficient to bind to MIF to enable detection of the MIF-compound complex such that diagnosis of a disease or condition is possible.

Suitable dosages may lie within the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another preferred embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage.

Suitable dosage amounts and dosing regimens can be determined by the attending physician or veterinarian and may depend on the desired level of inhibiting activity, the particular condition being treated, the severity of the condition as well as the general age, health and weight of the subject.

The active ingredient may be administered in a single dose or a series of doses. While it is possible for the active ingredient to be administered alone, it is preferable to present it as a composition, preferably as a pharmaceutical composition.

In a further aspect of the invention, there is provided a pharmaceutical composition comprising a compound of formula (I) or a pharmaceutically acceptable salt or prodrug thereof together with a pharmaceutically acceptable carrier, diluent or excipient.

The formulation of such compositions is well known to those skilled in the art. The composition may contain pharmaceutically acceptable additives such as carriers, diluents or excipients. These include, where appropriate, all conventional solvents, dispersion agents, fillers, solid carriers, coating agents, antifungal and antibacterial agents, dermal penetration agents, surfactants, isotonic and absorption agents and the like. It will be understood that the compositions of the invention may also include other supplementary physiologically active agents.

The carrier must be pharmaceutically acceptable in the sense of being compatible with the other ingredients of the composition and not injurious to the subject. Compositions include those suitable for oral, rectal, inhalational, nasal, transdermal, topical (including buccal and sublingual), vaginal or parenteral (including subcutaneous, intramuscular, intraspinal, intravenous and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Depending on the disease or condition to be treated, it may or may not be desirable for a compound of formula (I) to cross the blood/brain barrier. Thus the compositions for use in the present invention may be formulated to be water or lipid soluble.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg inert diluent, preservative, disintegrant (eg. sodium starch glycolate, cross-linked polyvinyl pyrrolidone, cross-linked sodium carboxymethyl cellulose)) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum; pastilles comprising the active ingredient in an inert basis such as gelatin and glycerin, or sucrose and acacia gum; and mouthwashes comprising the active ingredient in a suitable liquid carrier.

The compounds of formula (I) may also be administered intranasally or via inhalation, for example by atomiser, aerosol or nebulizer means.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal devices, such as patches, may also be used to administer the compounds of the invention.

Compositions for rectal administration may be presented as a suppository with a suitable carrier base comprising, for example, cocoa butter, gelatin, glycerin or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage compositions are those containing a daily dose or unit, daily sub-dose, as herein above described, or an appropriate fraction thereof, of the active ingredient.

It should be understood that in addition to the active ingredients particularly mentioned above, the compositions of this invention may include other agents conventional in the art having regard to the type of composition in question, for example, those suitable for oral administration may include such further agents as binders, sweeteners, thickeners, flavouring agents, disintegrating agents, coating agents, preservatives, lubricants and/or time delay agents. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

It will be recognised that other therapeutically active agents such as anti-inflammatory (eg steroids such as glucocorticoids) or anti-cancer agents may be used in conjunction with a compound of formula (I). Compounds of formula (I) when administered in conjunction with other therapeutically active agents may exhibit an additive or synergistic effect. These may be administered simultaneously, either as a combined form (ie as a single composition containing the active agents) or as discrete dosages. Alternatively, the other therapeutically active agents may be administered sequentially or separately with the compounds of the invention. Thus, the invention also relates to kits and combinations, comprising a compound of formula (I) and one or more other therapeutically active ingredients for use in the treatment of diseases or conditions described herein.

Without being limiting, examples of agents which could be used in combination with a compound of formula (I) include: glucocorticoids, antirheumatic drugs (including but not limited to methotrexate, leflunomide, sulphasalazine, hydroxycholorquine, gold salts); immunosuppressive drugs (including but not limited to cyclosporin, mycophenyllate mofetil, azathioprine, cyclophosphamide); anti-cytoline therapies (including but not limited to antagonists of, antibodies to, binding proteins for, or soluble receptors for tumor necrosis factor, interleukin 1, interleukin 3, interleukin 5, interleukin 6, interleukin 8, interleukin 12, interleukin 18, interleukin 17, and other pro-inflammatory cytokines as may be found relevant to pathological states); antagonists or inhibitors of mitogen-activated protein (MAP) kinases (including but not limited to antagonists or inhibitors of extracellular signal-regulated kinases (ERK), the c-Jun N-terminal kinases/stress-activated protein kinases (JNK/SAPK), and the p38 MAP kinases, and other kinases or enzymes or proteins involved in MAP kinase-dependent cell activation); antagonists or inhibitors of the nuclear factor kappa-B (NF-κB) signal transduction pathway (including but not limited to antagonists or inhibitors of NF-κB-kinase, interleukin receptor activated kinase, and other kinases or enzymes or proteins involved in NF-κB-dependent cell activation); antibodies, protein therapeutics, or small molecule therapeutics interacting with adhesion molecules and co-stimulatory molecules (including but not limited to therapeutic agents directed against intercellular adhesion molecule-1, CD40, CD40-ligand, CD28, CD4, CD-3, selectins such as P-selectin or E-selectin); bronchodilators such as β-adrenoceptor agonists or anti-cholinergics; antagonists of eicosanoid synthesis pathways such as non-steroidal anti-inflammatory drugs, cyclooxygenase-2 inhibitors, thromboxane inhibitors, or lipoxygenase inhibitors; antibodies or other agents directed against leukocyte surface antigens (including but not limited to antibodies or other agents directed against CD3, CD4, CD5, CD19, CD20, HLA molecules); agents used for the treatment of inflammatory bowel disease (including but not limited to sulphasalazine, mesalazine, salicylic acid derivatives); anti-cancer drugs (including but not limited to cytotoxic drugs, cytolytic drugs, monoclonal antibodies).

In another aspect, the invention provides a method of treating or preventing a disease or condition wherein MIF cytokine or biological activity is implicated comprising administering to a mammal a compound of formula (I) or a pharmaceutically acceptable salt or prodrug thereof and a second therapeutic agent.

In a preferred embodiment of the invention, the second therapeutic agent is a glucocorticoid compound. The mechanism through which MIF antagonises the effects of glucocorticoids has not been fully eludicated. Glucocorticoid effects on inflammation are dependent upon the transactivation of genes which exert inhibitory effects on cell activation, or on the transrepression of genes which exert stimulatory effects on cell activation. Transrepression effects are in part mediated via effects on intra-cellular signal transduction pathways such as the nuclear factor κB (NF-κB) and mitogen activated protein kinase (MAPK) pathways.

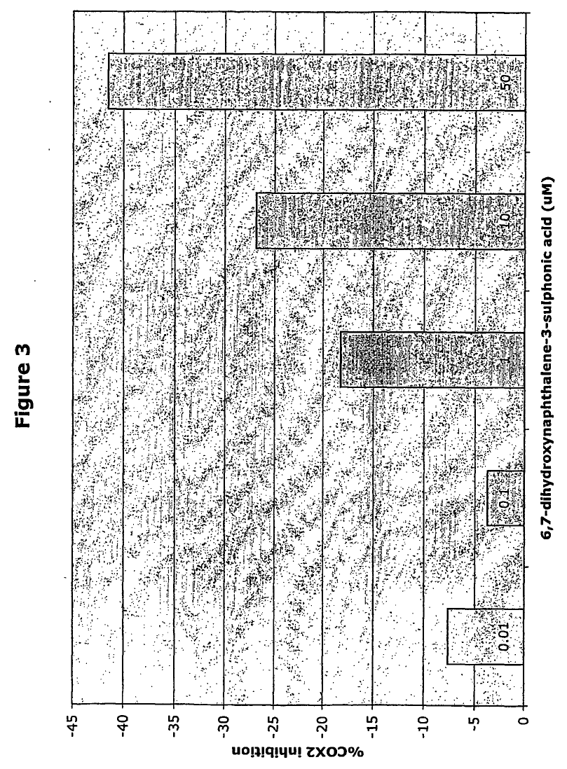

Without wishing to be bound by theory, it is possible that suppression of activation of signal transduction pathways by a MIF inhibitor may allow a glucocorticoid to be more effective. The ability of glucocorticoids to inhibit the activation of MAPK pathways is uncertain. Glucocorticoids have been variously reported either to suppress, or to be unable to suppress, MAPK activation under various conditions (15-17). Activation of the MAPK pathway known as ERK (extracellular signal regulated kinase, also known as p44/42 MAP kinase), as measured by the phosphorylation of ERK protein detected with a phospho-specific antibody, is increased by stimuli such as interleukin-1 (IL-1) (FIG. 3). The ERK pathway is also known to be activated by MIF (18). In experiments using human dermal fibroblasts, the glucocorticoid dexamethasone does not inhibit ERK pathway activation by IL-1. The combination of dexamethasone with a compound that inhibits the cytokine or biological activity of MIF, however, was able to inhibit ERK activation (FIG. 3).

Notwithstanding the incomplete understanding of the interacting pathways involved, it is possible that administration of a compound which inhibits the cytokine or biological activity of MIF in combination with a glucocorticoid exerts inhibitory effects on signal transduction pathways that are greater than the effects of the glucocorticoid alone. Where these signal transduction pathways are known to be important in the regulation of cell activation in conditions such as inflammatory diseases, it is likely that this greater effect would permit the use of lower doses of the glucococorticoid in a given patient; that is, the compound which inhibits the cytokine or biological activity of MIF would have a “steroid-sparing” effect.

In another aspect, the present invention provides a method of prophylaxis or treatment of a disease or condition for which treatment with a glucocorticoid is indicated, said method comprising administering to a mammal a glucocorticoid and a compound of formula (I) or a pharmaceutically acceptable salt or prodrug thereof.

In yet another aspect, the present invention provides a method of treating steroid-resistant diseases comprising administering to a mammal a glucocorticoid and a compound of formula (I) or a pharmaceutically acceptable salt or prodrug thereof.

In a further aspect, the present invention provides a method of enhancing the effect of a glucocorticoid in mammals comprising administering a compound of formula (I) or a pharmaceutically acceptable salt or prodrug thereof simultaneously, separately or sequentially with said glucocorticoid.

In yet a further aspect, the present invention provides a composition comprising a glucocorticoid and a compound of formula (I) or a pharmaceutically acceptable salt or prodrug thereof.

In a further aspect of the invention there is provided a use of a glucocorticoid in the manufacture of a medicament for administration with a compound of formula (I) or a pharmaceutically acceptable salt or prodrug thereof for the treatment or prophylaxis of a disease or condition for which treatment with a glucocorticoid is indicated.

In yet a further aspect of the invention there is provided a use of a compound of formula (I) or a pharmaceutically acceptable salt or prodrug thereof in the manufacture of a medicament for administration with a glucocorticoid for the treatment or prophylaxis of a disease or condition for which treatment of a glucocorticoid is indicated.

In yet a further aspect of the invention there is provided a use of a glucocorticoid and a compound of formula (I) or a pharmaceutically acceptable salt or prodrug thereof in the manufacture of a medicament for the treatment or prophylaxis of a disease or condition for which treatment with a glucocorticoid is indicated.

Preferably the amount of glucocorticoid used in the methods, uses and compositions of the invention is less than the amount which would be effective in the absence of the compound of formula (I). In the treatment of steroid-resistant diseases or conditions which are not responsive to glucocorticoids, any amount of glucocorticoid which is effective in combination with a compound of formula (I) is considered less than the amount which would be effective in the absence of a compound formula (I). Accordingly, the invention provides a steroid-sparing therapy.

In preferred embodiments of the invention, the glucocorticoid and the compound of formula (I) are used to treat or prevent a disease or condition in a mammal, preferably in a human subject.

The term “disease or condition for which treatment with a glucocorticoid is indicated” refers to diseases or conditions which are capable of being treated by administration of a glucocorticoid including but not limited to autoimmune diseases, solid or haemopoitic tumours, or chronic or acute inflammatory diseases. Examples of such diseases or conditions include:
Rheumatic diseases (including but not limited to rheumatoid arthritis, osteoarthritis, psoriatic arthritis, polymyalgia rheumatica) spondyloarthropathies (including but not limited to ankylosing spondylitis, reactive arthritis, Reiter's syndrome), crystal arthropathies (including but not limited to gout, pseudogout, calcium pyrophosphate deposition disease), Lyme disease, connective tissue diseases (including but not limited to systemic lupus erythematosus, systemic sclerosis, polymyositis, dermatomyositis, Sjögren's syndrome), vasculitides (including but not limited to polyarteritis nodosa, Wegener's granulomatosis, Churg-Strauss syndrome), glomerulonephritis, interstitial nephritis, inflammatory bowel disease (including but not limited to ulcerative colitis, Crohn's disease), peptic ulceration, gastritis, oesophagitis, liver disease (including but not limited to cirrhosis, hepatitis), autoimmune diseases (including but not limited to diabetes mellitus, thyroiditis, myasthenia gravis, sclerosing cholangitis, primary biliary cirrhosis), pulmonary diseases (including but not limited to diffuse interstitial lung diseases, pneumoconioses, fibrosing alveolitis, asthma, bronchitis, bronchiectasis, chronic obstructive pulmonary disease, adult respiratory distress syndrome), cancers whether primary or metastatic (including but not limited to colon cancer, lymphoma, lung cancer, melanoma, prostate cancer, breast cancer, stomach cancer, leukemia, cervical cancer, multiple myeloma and metastatic cancer), atherosclerosis (eg ischaemic heart disease, myocardial infarction, stroke, peripheral vascular disease), disorders of the hypothalamic-pituitary-adrenal axis, brain disorders (eg dementia, Alzheimer's disease, multiple sclerosis, demyelinating diseases), corneal disease, iritis, iridocyclitis, cataracts, uveitis, sarcoidosis, diseases characterised by modified angiogenesis (eg diabetic retinopathy, rheumatoid arthritis, cancer), endometrial function (menstruation, implantation, parturition, endometriosis), psoriasis, endotoxic (septic) shock, exotoxic (septic) shock, infective (true septic) shock, other complications of infection, pelvic inflammatory disease, transplant rejection, allergies, allergic rhinitis, bone diseases (eg osteoporosis, Paget's disease), atopic dermatitis, UV(B)-induced dermal cell activation (eg sunburn, skin cancer), malarial complications, diabetes mellitus, pain, inflammatory consequences of trauma or ischaemia, testicular dysfunctions and wound healing.

These diseases or conditions may also include steroid-resistant diseases or conditions where treatment with a glucocorticoid is indicated, but where the glucocorticoid is ineffective or is not as effective as expected.

Compounds of formula (I) may be particularly useful in combination with a glucocorticoid, for the treatment of a disease or condition selected from autoimmune diseases, or chronic or acute inflammatory diseases, including rheumatic diseases (including but not limited to rheumatoid arthritis, osteoarthritis, psoriatic arthritis, polymyalgia rheumatica) spondyloarthropathies (including but not limited to ankylosing spondylitis, reactive arthritis, Reiter's syndrome), crystal arthropathies (including but not limited to gout, pseudogout, calcium pyrophosphate deposition disease), connective tissue diseases (including but not limited to systemic lupus erythematosus, systemic sclerosis, polymyositis, dermatomyositis, Sjögren's syndrome), vasculitides (including but not limited to polyarteritis nodosa, Wegener's granulomatosis, Churg-Strauss syndrome), glomerulonephritis, interstitial nephritis, inflammatory bowel disease (including but not limited to ulcerative colitis, Crohn's disease), liver disease (including but not limited to cirrhosis, hepatitis), autoimmune diseases (including but not limited to diabetes mellitus, thyroiditis, myasthenia gravis, sclerosing cholangitis, primary biliary cirrhosis), pulmonary diseases (including but not limited to diffuse interstitial lung diseases, fibrosing alveolitis, asthma, bronchitis, bronchiectasis, chronic obstructive pulmonary disease, adult respiratory distress syndrome), cancers whether primary or metastatic (including but not limited to myeloma, lymphoma, lung cancer, leukemia, cervical cancer and metastatic cancer), atherosclerosis (eg ischaemic heart disease, myocardial infarction, stroke, peripheral vascular disease), disorders of the hypothalamic-pituitary-adrenal axis, brain disorders (including but not limited to multiple sclerosis, demyelinating diseases), corneal disease, iritis, iridocyclitis, uveitis, sarcoidosis, psoriasis, endotoxic (septic) shock, exotoxic (septic) shock, infective (true septic) shock, other complications of infection, transplant rejection, allergies, allergic rhinitis, bone diseases (including but not limited to osteoporosis), atopic dermatitis, malarial complications, inflammatory consequences of trauma or ischaemia, and wound healing.

The combination of glucocorticoid and compound of formula (I) may be particularly useful when used in a steroid-sparing manner. The term “steroid-sparing” refers to a combination therapy method that allows a reduction in the amount of glucocorticoid administered while still providing an effective therapy for the disease or condition being treated or prevented.

Steroid-resistant diseases or conditions are diseases or conditions for which treatment with a glucocorticoid is indicated, but where the glucocorticoid is ineffective or is not as effective as expected. This term encompasses diseases or conditions for which the effective dose of glucocorticoid results in unacceptable side effects and/or toxicity. Some steroid-resistant diseases or conditions may require a dosage of glucocorticoid so large that they are considered non-responsive and therefore are not able to be successfully treated with glucocorticoids. Some steroid-resistant diseases or conditions may require a large dosage of glucocorticoid to achieve only a small effect on the symptoms of the disease or condition. Furthermore, some patients, diseases or conditions present with symptoms that do not respond to treatment with a glucocorticoid, or may become less sensitive to glucocorticoid treatment over time. Examples of diseases which may commonly exhibit features of steroid-resistance include asthma, chronic obstructive pulmonary disease, rheumatoid arthritis, glomerulonephritis, systemic lupus erythematosus, inflammatory bowel disease and transplant rejection.

Glucocorticoids are a group of steroid hormones, which are used to treat or prevent a wide range of diseases or conditions. Suitable glucocorticoids may be synthetic or naturally occurring and include but are not limited to prednisolone, prednisone, cortisone acetate, beclamethasone, fluticasone, hydrocortisone, dexamethasone, methyl prednisolone, triamcinolone, budesonide and betamethasone. A person skilled in the art would be able to identify other suitable glucocorticoids that may benefit from being used in a combination treatment with a MIF antagonist.

In preferred embodiments of the invention, the glucocorticoid used is selected from prednisone, prednisolone, hydrocortisone, fluticasone, beclamethasone, betamethasone, methyl prednisolone, budesonide, triamcinolone, dexamethasone and cortisone. Most preferably, the glucocorticoid is selected from prednisone, prednisolone, methyl prednisolone, fluticasone and beclamethasone. Beclamethasone and fluticasone are particularly preferred for treating asthma. Prednisone, prednisolone and methyl prednisolone are particularly preferred in the treatment of systemic or local inflammatory diseases.

The amounts of glucocorticoid and compound of formula (I) are selected such that in combination they provide complete or partial treatment or prophylaxis of a disease or condition for which a glucocorticoid is indicated. The amount of compound formula (I) is preferably an amount that will at least partially inhibit the cytoline or biological activity of MIF. The amount of glucocorticoid is preferably less than the amount required in the absence of the compound of formula (I). The amounts of glucocorticoid and compound of formula (I) used in a treatment or therapy are selected such that in combination they at least partially attain the desired therapeutic effect, or delay onset of, or inhibit the progression of, or halt or partially or fully reverse the onset or progression of the disease or condition being treated. The amounts of glucocorticoid and compound of formula (I) used in the prophylaxis of a disease or condition are selected such that in combination they at least partially prevent or delay the onset of the disease or condition. Dosing may occur at intervals of minutes, hours, days, weeks, months or years or continuously over any one of these periods.

Suitable doses of a compound of formula (I) may lie within the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another preferred embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage.

Suitable dosage amounts of glucocorticoids will depend, in part, on the mode of administration and whether the dosage is being administered in a single, daily or divided dose, or as a continuous infusion. When administered orally, intravenously, intramuscularly, intralesionally or intracavity (eg. intra-articular, intrathecal, intrathoracic), dosages are typically between 1 mg to 1000 mg, preferably 1 mg to 100 mg, more preferably 1 mg to 50 mg or 1 mg to 10 mg per dose. When administered topically or by inhalation as a single, daily or divided dose, dosages are typically 1 ng to 1 μg, 1 ng to 1 mg or 1 pg to 1 μg.

Suitable dosage amounts and dosing regimens can be determined by the attending physician or veterinarian and may depend on the desired level of inhibiting activity, the particular condition being treated, the severity of the condition as well as the general age, health and weight of the subject.

The glucocorticoid and compound of formula (I) may be administered simultaneously or sequentially. The active ingredients may be administered alone but are preferably administered as a pharmaceutically acceptable composition or separate pharmaceutically acceptable compositions.

The formulation of such compositions is well known to those skilled in the art and are described above in relation to compounds of formula (I). The composition or compositions may contain pharmaceutically acceptable additives such as carriers, diluents or excipients. These include, where appropriate, all conventional solvents, dispersion agents, fillers, solid carriers, coating agents, antifungal and antibacterial agents, dermal penetration agents, surfactants, isotonic and absorption agents and the like. It will be understood that the compositions of the invention may also include other supplementary physiologically active agents.

Preferred unit dosage compositions are those containing a daily dose or unit, daily sub-dose, as herein above described, or an appropriate fraction thereof, of the glucocorticoids and/or compound of formula (I) which inhibit the cytokine or biological activity of MIF.

In one preferred aspect of the invention, the compounds of formula (I) may be administered together with, simultaneously or sequentially, glucocorticoids. In such a therapy, the amount of glucocorticoid required may be significantly reduced.

The compounds of formula (I), either as the only active agent or together with another active agent, eg. a glucocorticoid may also be presented for use in veterinary compositions. These may be prepared by any suitable means known in the art. Examples of such compositions include those adapted for:

(a) oral administration, external application (eg drenches including aqueous and non-aqueous solutions or suspensions), tablets, boluses, powders, granules, pellets for admixture with feedstuffs, pastes for application to the tongue;

(b) parenteral administration, eg subcutaneous, intramuscular or intravenous injection as a sterile solution or suspension; and

(c) topical application eg creams, ointments, gels, lotions, etc.

By virtue of their ability to bind to or antagonise MIF, compounds of formula (I) or salts or derivatives thereof may be used as laboratory or diagnostic or in vivo imaging reagents. Typically, for such use the compounds would be labelled in some way, for example, radio isotope, fluorescence or calorimetric labelling, or be chelator conjugated. In particular, compounds of formula (I) could be used as part of an assay system for MIF or as controls in screens for identifying other inhibitors. Those skilled in the art are familiar with such screens and could readily establish such screens using compounds of formula (I). Those skilled in the art will also be familiar with the use of chelate conjugated molecules for in vivo diagnostic imaging.

In yet a further aspect of the invention, there is provided a compound of formula (II) or a pharmaceutically acceptable salt or prodrug thereof:

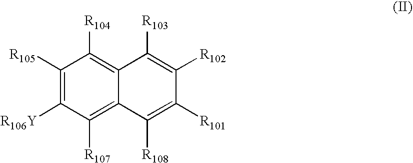

Wherein Y is selected from —O—, —NH—, —NC₁₋₃alkyl or —S(O)_{q}—

R₁₀₁ is selected hydrogen, C₁₋₆alkyl, CO₂H or CO₂C₁₋₆alkyl;

R₁₀₂ is selected from C₁₋₂₀alkyl, C₂₋₂₀alkenyl, CO₂H, CO₂C₁₋₂₀alkyl, CO₂C₂₋₂₀alkenyl, CO₂(CH₂)ₘR₁₀₉, SO₃H, SO₃C₁₋₂₀alkyl, SO₃C₂₋₃₀alkenyl, SO₃(CH₂)ₘR₁₀₉, C(O)C₁₋₂₀alkyl or (CH₂)ₘR₁₁₀;

R₁₀₃ is selected from hydrogen, hydroxy, methoxy or C₁₋₃alkyl;

R₁₀₄ is selected from hydrogen, C₁₋₃alkyl, NH₂, NH(C₁₋₃alkyl), N(C₁₋₃alkyl)₂ or (CH₂)ₘOH;

R₁₀₅ is selected from hydrogen, (CH₂)ₙOH or (CH₂)ₙOC₁₋₃alkyl;

R₁₀₆ is selected from hydrogen, C₁₋₃alkyl, C(O)NH₂, C(O)NH(C₁₋₃alkyl), C(O)N(C₁₋₃alkyl)₂, C(S)NH₂, C(S)NH(C₁₋₃alkyl) or C(S)N(C₁₋₃alkyl)₂;

R₁₀₇ is selected from hydrogen, hydroxy, halo, amino, nitro, cyano, SO₃H or CO₂H;

R₁₀₈ is selected from hydrogen or methyl;

R₁₀₉ is selected from halogen, hydroxy, C₁₋₃alkoxy, NH₂, NH(C₁₋₃alkyl), N(C₁₋₃alkyl)₂, CO₂H or CO₂C₁₋₃alkyl;

R₁₁₀ is selected from hydroxy, C₁₋₃alkyl, halo, CO₂H, CO₂C₁₋₃alkyl, CN, NH_{2,} NH(C₁₋₃alkyl) or N(C₁₋₃alkyl)₂;

n is 0 or an integer from 1 to 3;

m is 0 or an integer from 1 to 20; and

wherein an alkyl, alkenyl or alkyloxy, group may be optionally substituted one or more times.

Preferably the compounds of formula (II) are those in which at least one or more of the following definitions apply:

Y is selected from —O—, —S—, —NH—or SO₃;

R₁₀₁ is selected from hydrogen, CO₂H or CO₂C₁₋₃alkyl;

R₁₀₂ is selected from from C₁₋₂₀alkyl, C₂₋₂₀alkenyl, CO₂H, CO₂C₁₋₂₀alkyl, CO₂C₂₋₂₀alkenyl, CO₂(CH₂)ₘCO₂H, SO₃H, SO₃C₁₋₂₀alkyl, SO₃C₂₋₃₀alkenyl, SO₃(CH₂)ₘCO₂H, (CH₂)ₘhydroxy, (CH₂)ₘNH₂, (CH₂)ₘCN or (CH₂)ₘhalo;

R₁₀₃ is selected from hydrogen, hydroxy or methoxy;

R₁₀₄ is selected from hydrogen, hydroxy, methyl, NH₂ or CH₂OH;

R₁₀₅ is selected from hydrogen, hydroxy or methoxy,

R₁₀₆ is selected from hydrogen, C₁₋₃alkyl, C(O)NH₂, C(O)NH(C₁₋₃alkyl), C(O)N(C₁₋₃alkyl)₂, C(S)NH₂, C(S)NH(C₁₋₃alkyl) or C(S)N(C₁₋₃alkyl)₂;

R₁₀₇ is selected from hydrogen, hydroxy, halo, cyano, NH₂, nitro or SO₃H;

R₁₀₈ is hydrogen.

Preferred compounds of formula (I) include
6,7-dimethoxy-2-acetonoaphthone
2-carboxy-6-hydroxynaphthalene-5-sulfonic acid
Pentyl 6,7-dihydroxy-2-naphthalenesulfonate
2,3-dihydronaphtho[2,3-b][1,4]dioxine-7-carboxylic acid
Methyl 6-hydroxy-2-naphthoate
dodecanyl-6-hydroxy-2-naphthoate
[(6-hydroxy-2-naphthyl)carbonyl]oxyhexanoic acid
(6-methoxy-6-oxohexyl)-6-hydroxy-2-naphthoate
6-hydroxy-5-nitro-2-naphthoic acid
Ethyl 1,6-dihydroxy-2-naphthoate
Ethyl 6-[(dimethylamino)carbonyl]sulfanyl-1-methoxy-2-naphthoate
Ethyl 6-hydroxy-1-methoxy-2-naphthoate
Ethyl 6-[(dimethylamino)thiocarbonyl]oxy-1-methoxy-2-naphthoate
7-methoxy-3-hydroxy-2-naphthoic acid
Methyl 7-methoxy-3-hydroxy-2-naphthoate
Methyl 7-methoxy-3-methyl-2-naphthoate
7-methoxy-3-methyl-2-naphthoic acid
5-bromo-6-methoxy-2-methyl-3-naphthoic acid
6-hydroxy-[2-(1-pentylamino )methyl]-3-naphthoic acid
Methyl 3-bromomethyl-7-hydroxy-2-naphthoate
Methyl 7-methoxy-2-naphthoate
Methyl 7-hydroxy-2-naphthoate
Methyl 7-hydroxy-8-nitro-2-naphthoate
Methyl 6-hydroxy-5-nitro-2-naphthoate
Methyl 6-methoxy-5-nitro-2-naphthoate
Methyl 5-amino-6-methoxy-2-naphthoate
Methyl 6-methoxy-2-naphthoate.
2-hydroxymethyl-6-methoxynaphthalene
2-bromomethyl-6-methoxy-naphthalene
2-cyanomethyl-6-methoxynaphthalene
2-(1-cyano-1-hex-5-enyl)-6-methoxynaphthalene
2-(6-methoxy-2-naphthyl)hept-6-enoic acid
Methyl 2-(6-methoxy-2-naphthyl)hept-6-enoate
7-hydroxy-2-(6-methoxy-2-naphthyl)heptanoic acid
Methyl 6-methoxy-8-methyl-2-naphthoate.

Unless the context indicates otherwise, reference to any prior art in this specification is not, and should not be taken as, an acknowledgment or any form of suggestion that that prior art forms part of the common general knowledge in Australia.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

The invention will now be described with reference to the following examples which are included for the purpose of illustration only and are not intended to limit the generality of the invention hereinbefore described.

### EXAMPLES

Synthesis of compounds of Formula (I).

### Example 1

### 6 7-Dimethoxy-2-naphthalene

### 2,3-Dimethoxynaphthalene

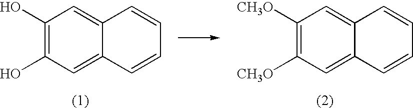

A suspension of 2,3-dihydroxynaphthalene (5.00g, 0.0312 mol) in water (25 mL) in a three-necked round-bottomed flask was cooled in an ice-bath. Two pressure equilibrating funnels were set up and these charged with dimethyl sulphate (7.20 mL, 9.57 g, 0.0759 mol) and aqueous potassium hydroxide (5.57 g, 0.0993 mol in 17.0 mL of water) respectively. Both of these were added together dropwise over 10 minutes resulting in the suspension first dissolving and then a precipitate forming. The reaction was left overnight at room temperature. The solid was then filtered off, washed with water until the washings were neutral (5×200 mL), and dried to give 2,3-dimethoxynaphthalene (4.09 g, 70% yield) as a white powder;

R_{f}: 0.71 (19:1 CHCl₃:MeOH), 0.82 (9:1 CHCl₃:MeOH), mp: 112-113° C., lit.mp:113-116° C.;

¹H NMR (CDCl₃/TMS): δ 4.01 (s, 6 H, 2×OCH₃), 7.13 (s, 2H), 7.33-7.36 (m, 2H), 7.68-7.71(m, 2H); LRESI mass spectrum: m/z 189 (100%, MH⁺).

### Example 2

### 6,7-Dimethloxy-2-acetonaphthone

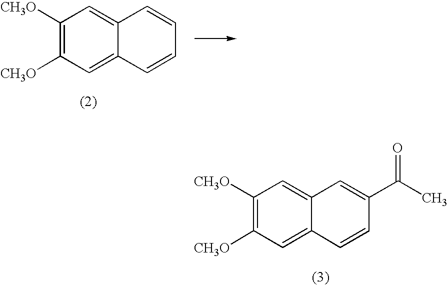

A suspension of aluminium chloride (6.02g, 0.0451 mol) in sieve-dried nitrobenzene (10 mL) was cooled in an ice-bath and acetyl chloride (3.57 mL, 3.93 g, 0.0501 mol) added over 5 minutes. 2,3-Dimethoxynaphthalene (7.52 g, 0.0400 mol) in nitrobenzenie (25 mL) was then added over 10 minutes. The reaction was stirred for a further 60 minutes at 0° C. and then left overnight at room temperature. The mixture was poured onto a mixture of ice (60 g) and 10% HCl (100 mL). Chloroform (300 mL) was added and the two phases separated. The aqueous was further extracted with chloroform (2×150 mL) and the combined organics then washed with 5% aqueous sodium hydroxide (3×100 mL) and water (2×100 mL), dried (anhydrous Na₂SO₄), filtered and evaporated under vacuo to give a brown oil. This was flash column chromatographed (silica gel, chloroform) to give 6,7-dimethoxy-2-acetonaphthone (8.51 g. 93% yield) as an orange solid. A sample was further recrystallised from ethanol to give fine orange needles;

R_{f}: 0.36 (CHCl₃), 0.62 (25:1 CHCl₃:MeOH), mp: 100-102° C., lit. mp: 113-116° C.;

¹H NMR (CDCl₃/TMS): δ 2.69 (s, 3H, COCH₃), 4.02 (s, 3H, OCH₃), 4.03 (s, 3H, OCH₃), 7.14 (s, 1H, H-8), 7.22 (s, 1H, H-5), 7.72 (d, 1H, J_{4,3} 8.4 Hz, H-4), 7.89 (dd, 1H, J_{3,1} 1.7 Hz, H-3), 8.33 (bs, 1H, H-1); LRESI mass spectrum: m/z 231 (100%, MH⁺).

### Example 3

### 6,7-Dimethoxy-2-naphthoic acid

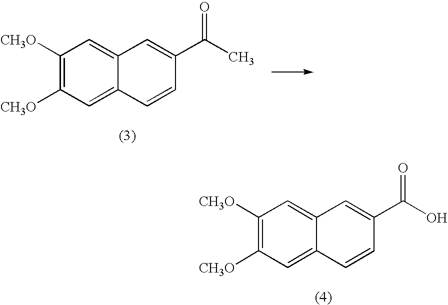

Sodium hypochlorite (55 mL, 12.5% w/v) was first added to sodium hydroxide (1.80 g, 0.0450 mole) dissolved in water (5.5 mL). This solution was gently heated to 45° C. and 6,7-dimethoxy-2-acetonaphthone (2.50 g. 0.0187 mole) then added. Heating was gradually increased until the suspension dissolved at a temperature of 85° C. and the solution was maintained at 85° C. for a further 60 minutes. The solution was then allowed to cool to room temperature and filtered to remove a small amount of orange gum. Small quantities of sodium bisulfite (spatulla ends) were then added to the filtrate until it no longer darkened iodine/starch indicator paper. The solution was then cooled in an ice-bath and concentrated Hcl added drop-wise until a pH of 1. The resultant white precipitate was filtered off, washed with cold water (3×20 mL) and dried under vacuum over a desiccant to give 6,7-dimethoxy-2-naphthanoic acid (2.2601 g, 90% yield as a white powder,

R_{f}: 0.36 (9:1 CHCl₃:MeOH), mp: 248-250° C.; lit. mp: 246-248° C.;

¹H NMR (CDCl₃/CD₃OD/TMS): δ 4.02 (s, 3H, OCH₃), 4.03 (s, 3H, OCH₃), 7.19 (s, 1H), H-8), 7.26 (s, 1H, H-5), 7.73 (d, 1H, J_{4,3} 8.5 Hz, H-4), 7.93 (dd, 1H, J_{3,1} 1.7 Hz, H-3), 8.47 (bs, 1H, H-1); LRESI mass spectrum: m/z 233 (41%, MH⁺), 255 (100%, MNa⁺).

### Example 4

### 2-carboxy-6-hydroxynaphthalene-5-sulfonic acid (5)

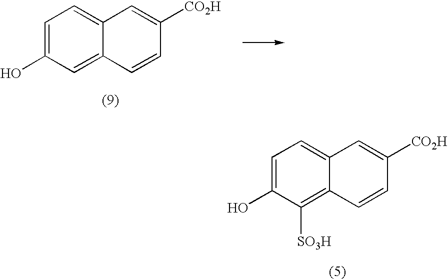

Conc. sulfuric acid (95-98%, 12 ml) was cooled in ice-bath and 6-hydroxy-2-naphthanoic acid (2.83 g; 15.05 mmol) added in small portions. The reaction mixture was stirred at room temperature for 4 hours. The white solid was filtered and recrystallised from water. This gave yield of 62%.

¹H NMR (DMSO-d6):δ 7.09 (d, 1H, Jₒᵣₜₕₒ=8.7 Hz, aromatic), 7.90 (d, 1H, Jₒᵣₜₕₒ=9.3 Hz, aromatic), 7.95 (d, 1H, Jₒᵣₜₕₒ=8.7 Hz, aromatic), 8.41 (s, 1H, aromatic) and 8.66 (d, 1H, Jₒᵣₜₕₒ=9.1 Hz, aromatic). Negative ion mass spectrum 267 m/z (100%).

### Example 5

### Pentyl 6,7-dihydroxy-2-naphthalenesulfonate (10)

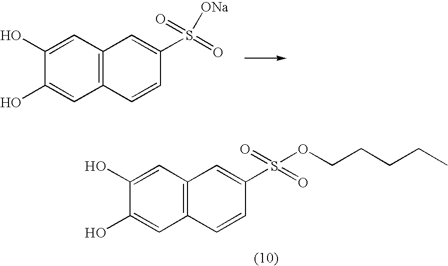

To a solution of the sulfonate (500 mg, 1.91 mmol) in anhydrous 1-pentanol (50 mL) was added Dowex H⁺ resin (500 mg). The mixture was refluxed for 42 hrs before filtering. Concentration of the solvent furnished a black gum. The gum was chromatographed on silica (hexanes/EtAc, 2:1) to furnish the title compound as a light brown solid (183 mg, 31%).

1H NMR (CDCl₃): δ 7.6-7.11 (bm, 5H, ArH), 4.2 (t, 2H, —OCH₂CH₂CH₂CH₂CH₃), 1.9 (m, 2H, —OCH₂CH₂CH₂CH₂CH₃), 1.4 (m, 4H, —OCH₂CH₂CH₂CH₂CH₃), 0.9 (t, 3H, —OCH₂CH₂CH₂CH₂CH₃); LRMS (ESI): m/z 311 [M+H⁺]; C₁₅H₁₈O₅S: 310.37

### Example 6

### 6-methylamino-2-naphthalene sulphonic acid, sodium salt (11)

Prepared by a procedure according to Cory et al. (19).

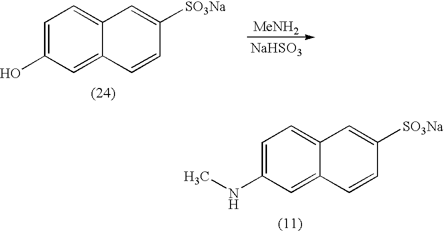

A mixture of sodium 6-hydroxy-2-naphthalene sulfonate 24 (1.00 g; 4.06 mmol), sodium bisulfite (3.6 g; 35 mmol), N-methylamine (2 M in THF; 19.9 mL; 40 mmol) and water (14 mL) was heated at reflux for 3 days before the aqueous phase was filtered through a plug of glass wool. Upon cooling to room temperature, the organic solution was treated with chloroform and the newly formed precipitate was collected by filtration. The white amorphous solid was crystallised from hot 1% aqueous NaOH solution and then recrystallised from water to afford a colourless crystalline solid (179 mg).

¹H NMR (d4-MeOH) δ 2.76 (3 H, d, J=5.0 Hz, CH₃), 6.05 (1H, q, J=5.0 Hz, NH), 6.65 (1H, d, J=2.1 Hz), 6.96 (1H, dd, J=8.8, 2.3 Hz), 7.50-7.57 (2H, m), 7.62 (1H, d, J=8.9 Hz), 7.91 (1H, s);

¹³C NMR δ 29.68 (CH₃), 101.55, 118.51, 124.01, 124.28, 124.77, 124.97, 128.96, 135.20, 140.55, 148.37; νₘₐₓ 3438 vs, 3371 vs, 1633 s, 1169 s, 1101 m, 1036 m cm⁻¹.

### Example 7

### 2,3-dihydronaphtho[2,3-b][1,4]dioxine-7-carboxylic acid (14)

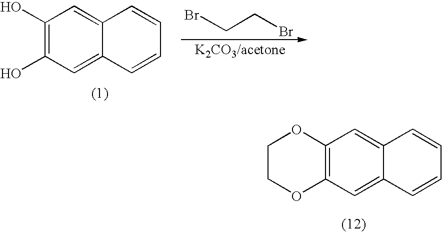

(a) Dry K₂CO₃ (12.17 g) and 1,2-dibromoethane (4.0 mL) were added to a solution of 2,3-dihydroxynaphthalene (5.0 g) in acetone (120 mL). The reaction mixture was heated under reflux for 24 h. The reaction mixture was cooled and diluted with ethyl acetate (100 mL) and the ethyl acetate layer was washed with brine. The organic layer was dried (Na₂SO₄) and evaporated to dryness to give the crude product which was purified by flash chromatography (ethyl acetate/hexane; 20:80). The dihydronaphthodioxin 12 was obtained as a white, shiny solid (3.5 g).

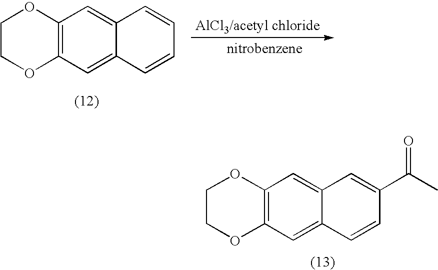

(b) The dihydronaphthodioxin 12 (0.75 g; 4.0 mmol) was dissolved in nitrobenzene (10 mL) and cooled to 0° C. Aluminium chloride (2.14 g; 16.1 mmol) was added portionwise. After 30 min, acetyl chloride (0.32 mL; 4.0 mmol) was added dropwise and stirring was continued for a further 30 minutes at 0° C. before ice-water (30 mL) was added slowly. The product was extracted into ether and the combined extracts were dried (Na₂SO₄) and evaporated to dryness. The nitrobenzene was removed by Kugelrohr distillation (100-110° C./2.5 mm). The resulting crude solid was triturated with ether to give the acetyl derivative 13 as an off-white solid (0.32 g).

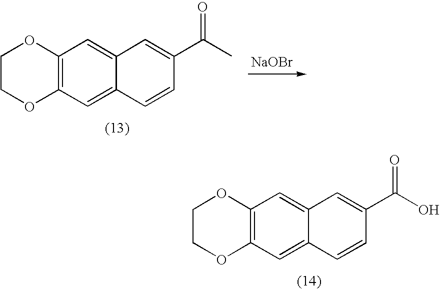

(c) The acid 14 was prepared by a procedure according to Bäckström et al. (20). Bromine (0.32 mL; 6.3 mmol) was added to a solution of NaOH (2.5 M; 8.5 mL) at 0° C. After 5 minutes the resulting solution was warmed to 35° C. and a suspension of the acetylated dioxin 13 (0.32 g; 1.4 mmol) in dioxane (4 mL) was added. Stirring was continued at 35° C. for a further 20 minutes before cooling to room temperature and adding sodium bisulfite (0.4 g) in water (3 mL). After 30 minutes at room temperature water (20 mL) was added and the reaction mixture was extracted with dichloromethane (20 mL). The aqueous layer was acidified to give a white precipitate which was collected by filtration. The acid 14 was obtained by trituration with warm methanol as a white solid (150 mg), m.p. 280-281° C.

¹H NMR (d₄-MeOH) δ 4.32 (4H, s), 7.24 (1H, s), 7.32 (1H, s), 7.62 (1H, d, J=8.5 Hz), 7.83 (1H, d, J=8.5 Hz), 8.32 (1H, s);

13C NMR δ 65.6, 65.8, 113.2, 114.6, 125.3, 126.9, 129.9, 130.1, 131.4, 132.7, 145.9, 146.9, 173.4 (C═O); νₘₐₓ 3430-3000 br s, 1707 s, 1698 s, 1520 m, 1280 s, 1197 s cm^{31 1}.

### Example 8

### Methlyl-6-hydroxy-2-naphthoate (15)

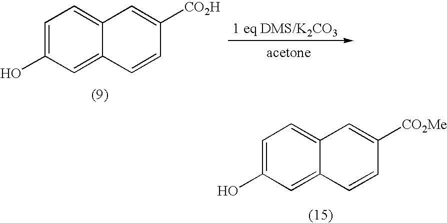

6-Hydroxy-2-naphthoic acid (2.0, 0.01 mol) was dissolved in acetone (100 mL), containing potassium carbonate (3.45 g, 0.0265 mmol) and then dimethyl sulfate (1.10 mL) was added dropwise. The reaction mixture was heated to reflux under nitrogen for 40 minutes and then cooled. Ammonium chloride (4%, 50 mL) was added. The aqueous layer was extracted with dichloromethane (3×40 mL) and the combined organic extracts were washed with ammonia solution (25%, 40 mL) and dried (Na₂SO₄). Evaporation of the solvent gave the crude ester 15 and this was triturated with 5% ethyl acetate/hexane and dichloromethane added dropwise, to give 15 as a white solid (1.75 g).

### Example 9

### Dodecanyl-6-hydroxy-2-naphthoate (19)

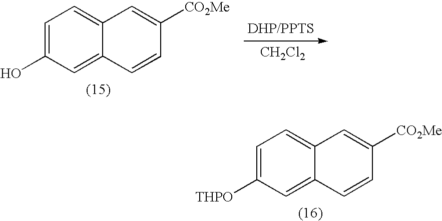

(a) The hydroxy ester 15 (0.5 g, 2.5 mmol) was dissolved in CH₂Cl₂ (15 mL) and cooled to 0° C. PPTS (20 mg) was added followed by DBP (0.25 mL, 2.7 mmol) added dropwise. The reaction mixture was left to stir at room temperature overnight after which time more DHP (0.25 mL) and PPTS (10 mg) were added. The reaction mixture was heated under reflux for 2 h. Upon cooling, water (40 mL) was added and the product was extracted into dichloromethane. The crude product was purified by flash chromatography (ethyl acetate, 40:60) to give the THP ether 16 as a white crystalline compound (0.8 g).

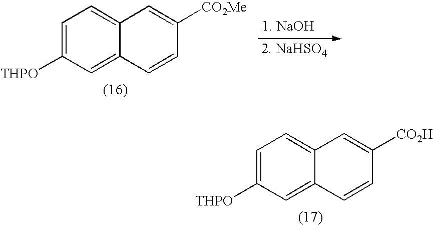

(b) The ester THP ether 16 (800 mg, 2.8 mmol) was dissolved in DME (40 mL) and cooled in an ice bath. KOH (1 M, 15 mL) was added slowly and the reaction mixture warmed to room temperature. Stirring was continued for 18 hours and then water (50 mL) was added before extracting with ether (50 mL) to remove impurities. The aqueous layer was cooled in ice and carefully neutralised with 1M NaHSO₄ (ca. 10 mL). The acid precipitated and was extracted with ethyl acetate (4×40 mL). The extracts were dried (Na₂SO₄), and evaporated to give the acid THP ether 17 as a white powder of sufficient purity to be used in the next step.

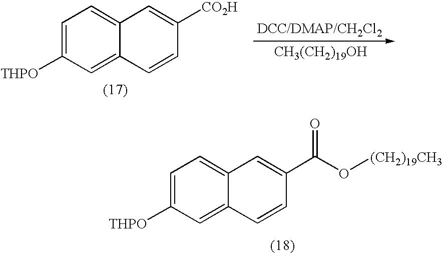

(c) A solution of the acid TBP ether 17 (0.20 g, 0.73 mmol), 1-eicosanol (0.20 g, 0.73 mmol) and DMAP (9 mg, 0.073 mmol) in dichloromethane (5 mL) was cooled to 0° C. A solution of DCC (0.17 g, 0.8 mmol), in CH₂Cl₂ (0.5 mL) was added dropwise. The reaction mixture was left to stir for 5 minutes and then allowed to warm to room temperature. Stirring was continued for 17 hours and then the reaction mixture was filtered and the dicyclohexylurea by-product was washed with dichloromethane. The filtrate was concentrated and the crude product purified by flash chromatography (ether/hexane, 60:40) to give the ester 18 as a white solid (250 mg).

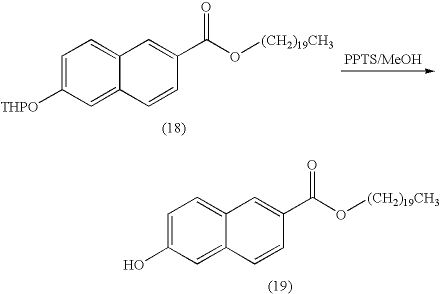

(d) To a solution of the ester THP ether 18 (0.23 g) in methanol (7 mL) was added PPTS (10 mg). The reaction mixture was heated under reflux for 2.5 h. The MeOH was removed by evaporation, water (15 mL) and dichloromethane (20 mL) were added and the whole was shaken. The organic layer was separated and the aqueous layer was was extracted into dichloromethane (3×10 mL). The combined extracts were dried (Na₂SO₄) and evaporated to dryness. The crude product was purified by flash chromatography (ether/hexane, 70:30) and trituration with ether/hexane (30:70) to give 19 as a white solid (130 mg), m.p. 103-104° C.;

1H NMR (CDCl₃) δ 0.88 (3H, t, J=7.0 Hz), 1.23-1.60 (34H, m), 1.81 (2H, quin, J=6.7 Hz), 4.36 (2H, t, J=6.7 Hz), 5.56 (1H, br s, W_{h/2}=7.5 Hz), 7.13-7.19 (2H, m), 7.69 (1H, d, J=8.6 Hz), 7.86 (1H, d, J=8.6 Hz), 8.01 (1H, dd, J=8.6, 1.6 Hz), 8.52 (1H, s);

¹³C NMR δ 14.1, 22.7, 26.1, 28.8, 29.3, 29.4, 29.5, 29.6 (10° C), 29.7, 31.9, 65.2, 109.4, 118.6, 125.6, 126.0, 126.4, 127.9, 130.9, 131.5, 137.1, 155.5, 167.1 (C═O); νₘₐₓ (KBr) 3402 s, 1684 s, 1297 m, 1210 m cm⁻¹.

### Example 10

### [6-hydroxy-2-naphtyl)carbonyl]oxyhexatioic acid (22)

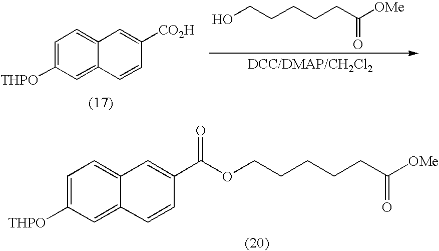

(a) A solution of the acid THP ether 17 (0.20 g, 0.73 mmol), methyl 6-hydroxyhexanoate (0.11 g, 0.73 mmol), and DMAP (9 mg, 0.073 mmol) in dichloromethane (5 mL) was cooled to 0° C. and then a solution of DCC (0.17 g, 0.8 mmol) in CH₂Cl₂ (0.5 mL) was added dropwise. The reaction mixture was left to stir for 5 minutes before warming to room temperature. After 17 hours the dicyclohexylurea was filtered off washing with dichloromethane. The filtrate was concentrated and the crude product purified by flash chromatography (ether/hexane, 60:40) to give the ester 20 as a white solid (160 mg).

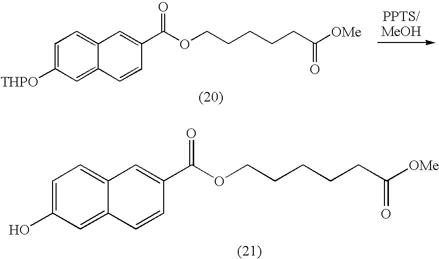

(b) To a solution of the ester THP ether 20 (0.14 g) in methanol (7 mL) was added PPTS (10 mg). The reaction mixture was heated under reflux for 2.5 h. The MeOH was removed by evaporation, water (15 mL) and dichloromethane (20 mL) were added and the whole was shaken. The organic layer was separated and the aqueous layer was extracted into dichloromethane (3×10 mL). The combined extracts were dried (Na₂SO₄) and evaporated to dryness. The crude product trituration with ether/hexane (30:70) to give (6-methoxy-6-oxohexyl)-6-hydroxy-2-naphthoate 21 as a white solid (100 mg).

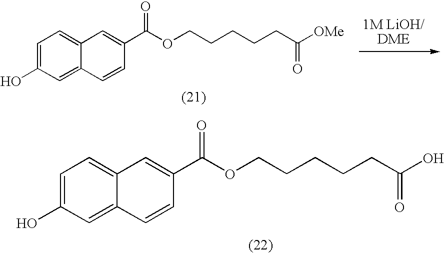

c) The hydroxy ester (80 mg) was dissolved in DME (8 mL) and treated with lithium hydroxide (2 mL, 1 M) dropwise at room temperature. Stirring was continued for 4 hours whereupon water (5 mL) was added and the reaction mixture was acidified to pH 4/5, with HCl (1 M). The product was then extracted into dichloromethane, dried (Na₂SO₄) and evaporated to dryness. The crude product was purified by flash chromatography (ether/hexane/CH₃COOH, 85:15: 1), to give 22 as a white solid (50 mg), m.p. 133-134° C.;

¹H NMR (d₄-MeOH) δ 1.50-1.87 (6H, m), 2.35 (2H, t, J=7.2 Hz), 4.35 (2H, t, J=6.5 Hz), 7.11-7.16 (2H, m), 7.69 (1H, d, J=8.7 Hz), 7.84-7.88 (1H, m), 7.92 (1H, dd, J=8.7, 1.7 Hz), 8.47 (1H, s);

¹³C NMR δ 25.8, 26.8, 29.6, 34.9, 65.9, 109.9, 120.4, 125.7, 126.4, 127.4, 128.6, 131.9, 132.2, 139.1, 159.1, 168.6 (C═O), 177.7 (C═O); νₘₐₓ 3068 w, 3053 w, 1689 w, 1614 m, 1587 m, 1510 s, 1477s, 1290s, 1245 s cm⁻¹.

### Example 11

### 6-hydroxy-5-nitro-2-napthoic acid (23)

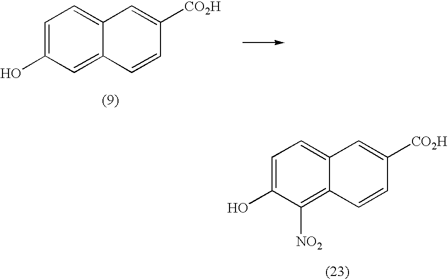

A solution of conc. sulfuric acid (0.27 ml) and water (0.80 ml) was cooled in an ice-bath and sodium nitrate (300 mg, 0.004 mole) added. The solution was left to stir until no solid was observed, then 6-hydroxy-2-naphthoic acid (400 mg, 0.002 mmole) was added. The solution was firstly stirred for 10 minutes in ice and then stirred for a further 3 hr at room temperature. Water (20 ml) was added and the solid filtered off. The compound was chromatographed over silica gel and eluted with 4:1 CHCl₃ and MeOH to yield 349 mg of 23.

Negative ion ESI MS: M/Z 232.024609 (MH).

### Example 12

### Ethyl 1,6-dihydroxy-2-naphthaote (28)

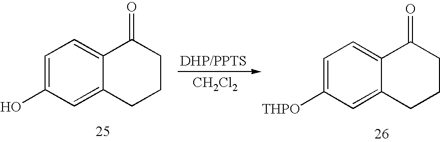

(a) 6-Hydroxytetralone 25 (2.0 g; 12.3 mmol) was suspended in dichloromethane (100 mL) and stirred in the presence of 3,4-dihydropyran (3.11 g; 37 mmol; 3.38 mL) and PPTS (100 mg) for 3.5 days. The organic layer was washed with water and brine and dried (Na₂SO₄). The solid remaining after removing the solvent was purified by flash chromatography (ether/hexane; 20:80) to give the tetrahydropyran 26 as an off-white solid (2.67 g; 88%).

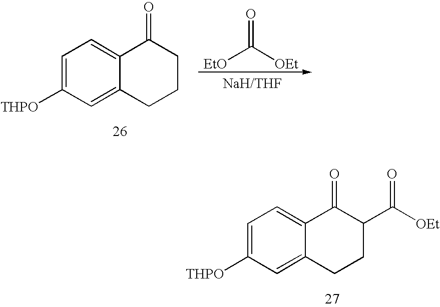

(b) The tetrahydropyran 26 (0.90 g; 3.7 mmol) was dissolved in TIF (10 mL) together with diethyl carbonate (0.86 g; 7.3 mmol; 0.88 mL). Sodium hydride (0.39 g; 16 mmol; 60% dispersion in oil) was added portionwise with stirring at room temperature and then the reaction mixture was heated under reflux for a further 17 hours. The resulting brown mixture was cooled, treated with acetic acid (17 M, 0.6 mL) and extracted with ether. The ether extracts were washed with brine and dried.(Na₂SO₄). Evaporation of the solvents left an orange viscous oil. This was purified by flash chromatography (ether/hexane; 40:60) to give the ketoester 27 as a yellow waxy solid (1.0 g).

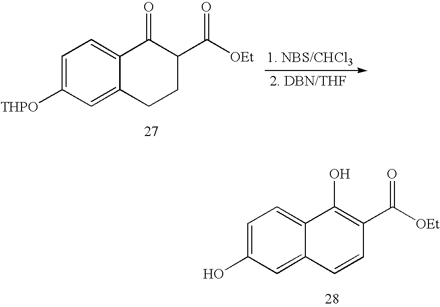

(c) Aromatisation was conducted according to a literature procedure (21). The ketoester 27 0.53 g; 1.66 mmol) was dissolved in chloroform (5 mL) and then N-bromosuccinimide (0.32 g; 1.83 mmol) and a few crystals of AIBN were added. The reaction mixture was heated under reflux for 40 minutes before being allowed to cool and diluting it with hexane (5 μL). Succinimide precipitated out and was removed by filtration. The filtrate was evaporated to dryness and then the residue was redissolved in anhydrous THF (2.5 mL). While stirring under a slow stream of nitrogen, DBN (0.40 mL; 3.32 mmol) was added dropwise and then the resulting solution was stirred overnight. During this time a precipitate formed. The reaction mixture was cooled in ice, diluted with ether, treated with acetic acid (17 M; 0.3 mL) and extracted with ether. The combined extracts were dried (Na₂SO₄) and evaporated to dryness to give a brown oil. The dihydroxynaphthoate 28 was obtained by flash chromatography (ether/hexane; 20:80) as a white solid (76 mg).

¹H NMR (CDCl₃/d₄-MeOH, 5:1) δ 1.43 (3H, t, J=7.1 Hz), 4.42 (2H, q, J=7.1 Hz), 7.06-7.11 (3H, m), 7.68 (1H, d, J=8.9 Hz), 8.26 (1H, d, J=8.9 Hz);

¹³C NMR δ 14.09, 61.05, 103.53, 109.36, 117.11, 117.31, 118.81, 124.82, 125.72, 139.32, 157.91, 160.79, 171.05 (C═O); νₘₐₓ 3386-3485 br m, 1684 m, 1653 s, 1559 s, 1507 s, 1273 s cm⁻¹.

### Example 13

### Ethyl-6-[(dimethylamino)carbonyl]sulfanyl-1-methoxy-2-naphthoate (33)

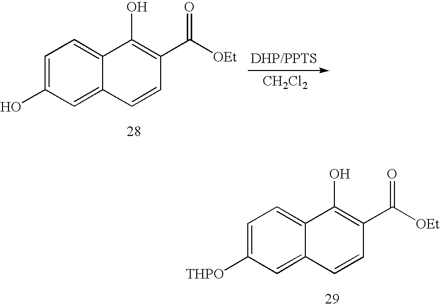

(a) The dihydroxynaphthoate 28 (76 mg; 0.33 mmol) was dissolved in dichloromethane (3 mL) and treated with dihydropyran (45 μL; 0.49 mmol) and a few crystals of PPTS. The reaction mixture was stirred for 3 days, diluted with ether and washed with water. The aqueous layer was further extracted with ether and the combined extracts were dried (Na₂SO₄) and evaporated to dryness. Flash chromatography (ether/hexane; 10:90) afforded the THP ether 29 (55 mg). This was used immediately in the next step.

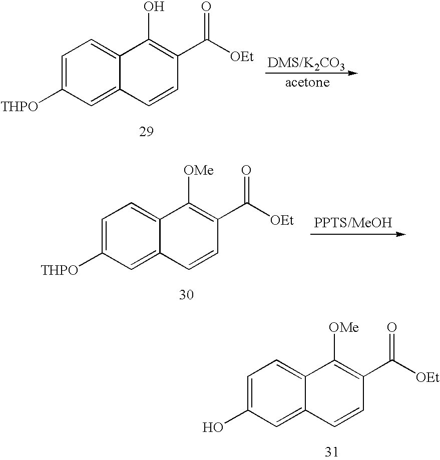

(b) The THP ether 29 (55 mg; 0.17 mmol) was dissolved in acetone (5 mL) and heated under reflux with dimethyl sulfate (25 μL; 0.26 mmol) and potassium carbonate (48 mg; 0.35 mmol) for 2.5 hours. The reaction mixture was allowed to cool, poured onto 25% ammonia solution and extracted with ether. The ether extracts were dried (Na₂SO₄) and evaporated to dryness. The crude methyl ether 30 was immediately submitted to hydrolysis conditions by dissolving in methanol (5 mL) and heating under reflux in the presence of catalytic PPTS for 3 hours. The reaction mixture was diluted with water and extracted with ether. Drying and evaporation of the solvent left the hydroxymethoxynaphthoate 31 as a white solid (38 mg) which was not purified.

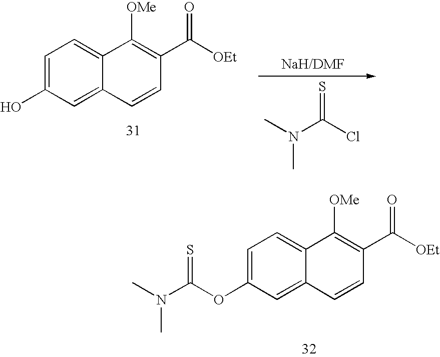

(c) Introduction of latent thiol functionality at the 6-position was conducted according to a literature procedure (22). The hydroxymethoxynaphthoate 31 (38 mg; 0.15 mmol) was dissolved in anhydrous DMF, cooled in ice and treated all at once with sodium hydride (42 mg; 0.17 mmol; 60% dispersion in oil). After H₂ evolution had ceased the yellow mixture was stirred for a further 15 minutes and then while cooling, dimethylcarbamoyl chloride (0.214 g; 0.17 mmol) was added all at once. The reaction mixture was stirred at ca. 30° C. for 1 hour becoming green in colour and then blue. The reaction mixture was quenched with water while cooling in ice and extracted with ether. The ether extracts were dried (Na₂SO₄) and evaporated to dryness. Flash chromatography (ether/hexane; 50:50) afforded the O-aryl thiocarbamate 32 as a white crystalline solid (25 mg).

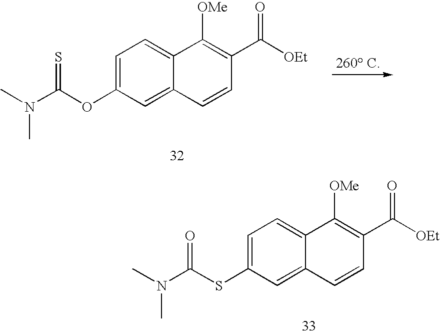

(d) The O-aryl thiocarbamate 32 (25 mg) in a 25 mL round bottom flask was submerged in a sand bath heated to 260° C. for 2 hours while a slow stream of nitrogen was passed over it. Rearrangement proceeded to greater than 50% conversion to give 33 as a compound more polar than 32. Chromatography (ether/hexane; 1:1) afforded the S-aryl thiocarbamate 33 as an oil that crystallised on standing (11 mg).

¹H NMR (CDCl₃) δ1.45 (3H, t, J=7 Hz), 3.05 (3H, br s), 3.14 (3H, br s), 4.05 (3H, s), 4.45 (2H, q, J=7 Hz), 7.58 (1H, d, J=8.6 Hz), 7.62 (1H, dd, J=8.8, 1.7 Hz), 7.87 (1H, d, J=8.6 Hz), 8.02 (1H, d, J=1.7 Hz), 8.26 (1H, d, J=8.8 Hz);

¹³C NMR δ 14.32, 36.99 (2C), 61.22, 63.47, 119.99, 120.51, 123.53, 124.15, 127.41, 128.50, 129.37, 132.80, 134.81, 136.69, 157.99, 166.13 (C═O), 166.43 (C═O); νₘₐₓ 1699 s, 1654 s, 1333 m, 1272 m, 1249 m, 1137 m cm⁻¹.

### Example 14

### 7-methoxy-3-methyl-2-naphthoic acid: 2-amino-2-methylpropan-1-ol salt (39)

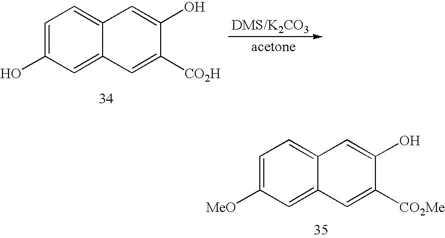

(a) The following represents an improvement on a previously reported procedure (23). Dimethyl sulfate (11.1 g; 88 mmol; 8.4 mL) was added slowly to a stirred suspension of 2,6-dihydroxy-3-naphthoic acid 34 (9.0 g; 44 mmol) and potassium carbonate (12.0 g; 92.4 mmol) in acetone (150 mL). The reaction mixture was heated under reflux for 21 hour with more dimethyl sulfate being added after 2 hours (2.1 mL; 11 mmol) and after 4 hours (2.1 mL; 11 mmol). The reaction mixture was poured onto water and extracted into dichloromethane. The dichloromethane layer was dried (Na₂SO₄) and evaporated to dryness to give a yellow solid. This was recrystallised from methanol (300 mL) to give methyl 2-hydroxy-6-methoxy-3-naphthoate 35 (24) as yellow needles (6.19 g). A second crop (1.94 g) was obtained from the mother liquor.

¹H NMR (CDCl₃) δ 3.8 (3H, s), 4.01 (3H, s), 7.07 (1H, d, J=2.4 Hz), 7.19 (1H, dd, J=9.0, 2.6 Hz), 7.26 (1H, s),7.58 (1H, d, J=9.0 Hz), 8.36 (1H, s), 10.26 (1H, s);

¹³C NMR δ 52.6, 55.4, 106.4, 112.0, 114.4, 122.8, 127.9 (2C), 130.7, 133.8, 155.1, 156.3, 170.5 (C═O); νₘₐₓ 3500-3100 br s, 1687 s, 1679s, 1519 s, 1292 vs, 1236 vs, 1081s, 1028 s cm⁻¹.

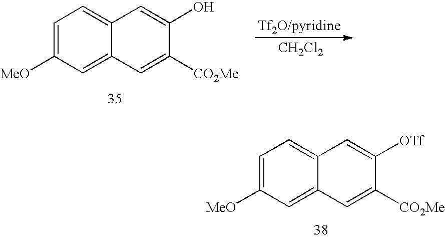

(b) Methyl 2-hydroxy-6-methoxy-3-naphthoate 35 (3.5 g; 15 mmol) was dissolved in pyridine (10 mL) and treated slowly with trifluoromethanesulfonic anhydride (4.7 g; 16.5 mmol; 2.8 mL) at 0° C. Stirring was continued at this temperature for a further 30 minutes and then the reaction mixture was allowed to warm to room temperature. After 4.5 hours water (50 mL) was added and the mixture was extracted with ether. The combined extracts were dried (Na₂SO₄) and the solvent removed by evaporation. The pyridine was removed under high vacuum and the resulting viscous oil was crystallised in the freezer overnight. The brown crystals so obtained were triturated with hexane/ethyl acetate to give the triflate 36 as a pale yellow solid (4.2 g).

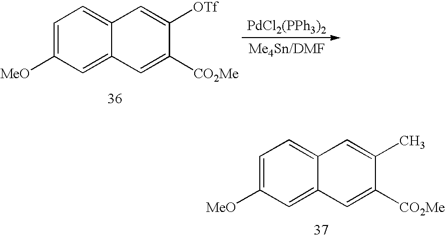

(c) The 2-methyl substituted naphthalene was prepared by modification of a related procedure (25). The triflate 36 (0.5 g; 1.4 mmol), anhydrous lithium chloride (0.49 g; 11.5 mmol), triphenylphosphine (0.216 g; 0.82 mmol), PdCl₂(PPh₃)₂ (60 mg; 0.86 mmol) and a few crystals of BHT were stirred in anhydrous degassed DMP (7 mL) under argon at 85° C. Tetramethyltin (0.736 g; 4.12 mmol; 0.57 mL) was added dropwise. After 2.5 hours more tetramethyltin (0.736 g; 4.12 mmol; 0.57 mL) was added. Stirring was continued for a total of 21 hours ensuring that the reaction temperature was maintained at 80-90° C. After this time the reaction mixture was cooled, diluted with water and extracted with dichloromethane. The organic layer was washed with saturated potassium fluoride, and brine, and dried (Na₂SO₄). Evaporation of the solvent left a crude solid that was purified by flash chromatography (ethyl acetate/hexane, 10:90). Methyl 6-methoxy-2-methyl-3-naphthoate 37 was obtained as white crystals (180 mg).

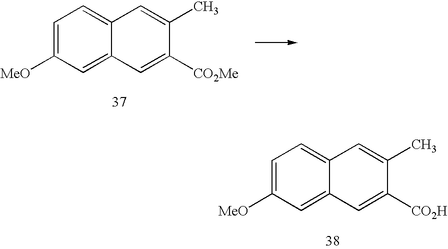

(d) 1 M NaOH (2 mL) was added slowly to a stirred solution of the ester 37 (210 mg; 0.912 mmol) in acetonitrile (7 mL). The reaction mixture was then heated under reflux for 2.5 hours and stirred at room temperature for a further 15 hours. Water (20 mL) was added and the whole was extracted with dichloromethane (20 mL). The aqueous layer was acidified with 3 M HCl and then the product was extracted into dichloromethane (3×30 mL). The combined extracts were dried (Na₂SO₄) and evaporated to dryness to give the acid 38 as a white solid (185 mg) that did not require further purification.

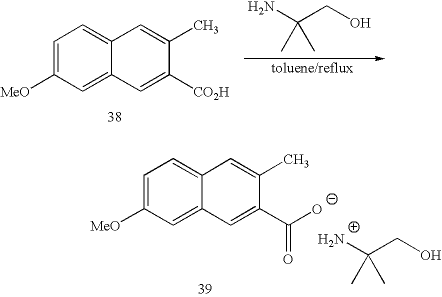

(e) The acid 38 (350 mg; 1.62 mmol) and 2-amino-2-methyl-1-propanol (231 μL; 2.43 mmol) were heated in toluene (10 mL) under reflux for 16 hours and then the toluene was removed under reduced pressure. The resulting solid was triturated with hexane/ether to give the ammonium salt 39 (465 mg) as an off-white solid.

¹H NMR (d₄-MeOH) 1.27 (6H, s,), 2.56 (3H, s), 3.46 (2H, s), 3.87 (3H, s, OMe),7.06 (1H, dd, J=8.9, 2.5 Hz), 7.18 (1H, d, J=2.5 Hz), 7.52 (1H, s), 7.62 (1H, d, J=8.9 Hz), 7.83 (1H, s);

¹³C NMR δ 20.72, 22.81 (2C), 55.70, 55.91, 68.20, 106.77, 119.87, 126.15, 128.93, 129.36, 130.34, 131.69, 134.09, 141.16, 158.73, C═O not visible; νₘₐₓ 3200-2000 br vs, 1607 m, 1560 s, 1542 s, 1363 s, 1227 m, 1200 m cm⁻¹.

### Example 15

### 5-bromo-6-methoxy-2-methyl-3-naphthaoic acid (40)

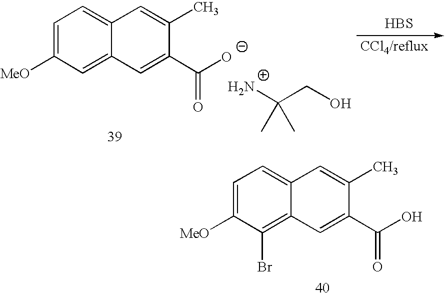

The ammonium salt 39 (200 mg; 0.66 mmol), N-bromosuccinimide (150 mg; 0.85 mmol) and dibenzoyl peroxide (2 mg) were heated in carbon tetrachloride (10 mL) under reflux for 4 hours. Upon cooling the resulting solid was filtered off and found to contain the product and succinimide, with more product being in the filtrate. The solid was triturated with ether/hexane and methanol added dropwise to give the bromide as an off-white solid 40 (80 mg). Further purification was achieved by flash chromatography (ethyl acetate/hexane, 45:55).

¹H NMR (d₆-DMSO) δ 2.63 (3H, s), 3.99 (3H, s), 7.60 (1H, d, J=9.0 Hz) 7.83 (1H, s), 7.96 (1H, d, J=9.0 Hz), 8:60 (1H, s);

¹³C NMR (CDCl₃) δ 21.0, 56.95, 107.30, 116.50, 128.24, 128.37, 129.97, 130.07, 130.56, 131.13, 133.05, 153.62, 168.52 (C═O); νₘₐₓ 3200-2000 br vs, 1684 s, 1259 s cm⁻¹.

### Example 16

### 6-hydroxy-[2-(1-pentylamino)methyl]-3-naphthoic acid (43)

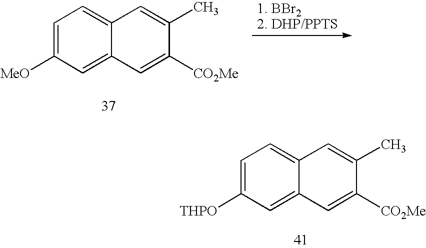

(a) The methoxy ester 37 (280 mg; 1.22 mmol) in dichloromethane (7 mL) was cooled in ice and treated with BBr₃ (2.43 mL; 2.43 mmol; 1 M in hexane) dropwise. After 30 minutes water (20 mL) was added and the reaction mixture was extracted with dichloromethane. The combined extracts were dried (Na₂SO₄) and evaporated to dryness. The resulting solid was triturated with hexane and ether added dropwise to give methyl 6-hydroxy-2-methylnaphthoate as a white solid (170 mg; 0.79 mmol). This was heated under reflux with DHP (0.16 mL; 1.75 mmol) and PPTS (10 mg) in dichloromethane (7 mL) for 15 hours. After this time water (20 mL) was added and the mixture was extracted with dichloromethane. The combined extracts were dried (Na₂SO₄) and evaporated to dryness to give the crude THP ether. The ester TBP ether 41 was isolated by flash chromatography (ethyl acetate/hexane, 15:85) as a colourless oil that solidified on standing (100 mg).

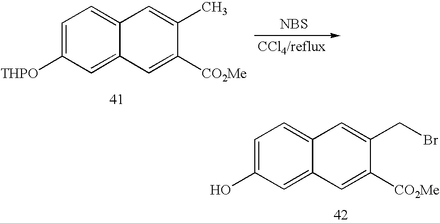

(b) The ester THP ether 41 (100 mg; 0.33 mmol), N-bromosuccinimide (71 mg; 0.40 mmol) and dibenzoyl peroxide (1 mg) were heated in carbon tetrachloride (5 mL) under reflux for 4 hours. After this time the reaction mixture was diluted with dichloromethane (30 mL) and washed with water (30 mL). The aqueous layer was extracted with dichloromethane and the combined extracts were dried (Na₂SO₄) and evaporated to dryness. The 2-bromomethyl derivative 42, resulting from concomitant benzylic bromination and deprotection of the 6-hydroxyl, was isolated by flash chromatography (ethyl acetate/hexane, 30:70) as a white solid (40 mg).

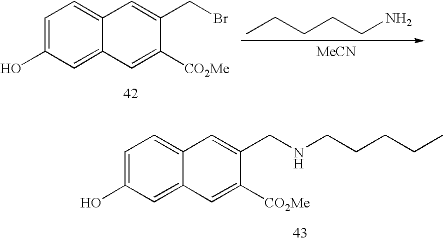

(c) The bromide 42 (26 mg; 0.088 mmol) and pentylamine (400 μL) were dissolved in anhydrous acetonitrile (2.5 mL) and heated at 60° C. for 3 days. After this time the solvent was removed under reduced pressure and the crude solid was purified by flash chromatography (ethyl acetate/hexane, 40:60) to give and amine 43 as a white solid (23 mg).

¹H NMR (CDCl₃) δ 0.80-0.98 (3H, m), 1.32-1.45 (4H, m), 1.57-1.70 (2H, m), 3.44 (2H, q, J=6.8 Hz), 3.89 (3H, s), 4.62 (2H, d, J=6.0 Hz), 4.70-4.80 (1H, m), 6.70-6.85 (1H, m), 7.08 (1H, d, J=2.5 Hz), 7.19 (1H, dd, J=8.9, 2.5 Hz), 7.62 (1H, s), 7.67 (1H, d, J=8.9 Hz), 7.85 (1H, s);

¹³C NMR δ 14.12, 22.49, 29.27, 29.37, 40.42, 55.46, 65.02, 106.15, 120.67, 126.92, 129.34, 129.66 (2C), 133.48, 134.34, 134.54, 158.49, 170.45 (C═O); νₘₐₓ 3360-3140 br s, 3140-3000 br s, 1624 vs, 1559 s, 1206 s, 1031 m, 1016 m cm⁻¹.

### Example 17

### Methyl 7-methoxy-2-naphthoate ester (44)

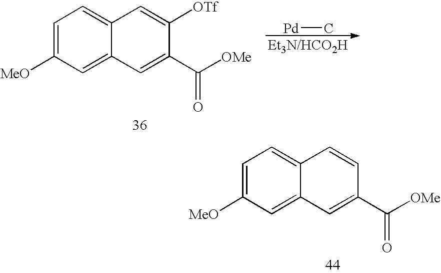

Ester (44) was prepared according to a related literature procedure (26). To a stirred solution of the aryl triflate 36 (0.5 g; 1.37 mmol) in anhydrous DMF (7 mL) under argon were added sequentially, triethylamine (0.765 mL; 5.49 mmol), formic acid (0.207 mL; 5.49 mmol), PPh₃ (72 mg; 0.27 mmol), and Pd(OAc)₂ (15.4 mg; 0.069 mmol). The reaction mixture was heated at 60° C. (bath) for 3.5 hours, after which time dichloromethane (40 mL) was added and the whole was washed with 5% HCl (2×20 mL) until pH 7, and water (30 mL). The organic layer was dried (Na₂SO₄) and evaporated to dryness. The crude product was isolated by flash chromatography (ether/hexane, 40:60) to give methyl 7-methoxy-2-naphthoate 44 as a yellow solid (190 mg);

¹H NMR (CDCl₃) δ 3.93 (3H, s), 3.97 (3H, s), 7.23 (1H, s), 7.24 (1H, d, J=8.5 Hz), 7.77 (1H, d, J=8.5 Hz), 7.80 (1H, d, J=8.6 Hz), 7.92 (1H, dd, J=8.5, 1.6 Hz), 8.50 (1H, s);

¹³C NMR δ 52.3, 55.5, 107.0, 121.4, 123.2, 128.0, 129.3, 129.9, 131.3, 133.9, 158.3, 167.5 (C═O) (note: one 4° aromatic carbon obscured), νₘₐₓ 1717 s, 1608 m, 1517 m, 1286 s, 1220 s, 1099 m cm⁻¹.

### Example 18

### Methyl 7-hydroxy-2-naphanoate ester (45)

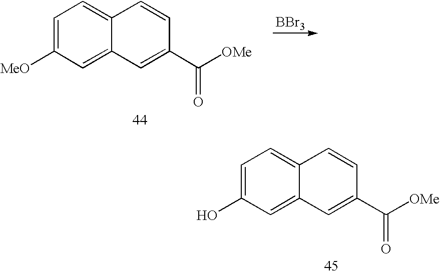

The ester 44 (0.39 g; 1.80 mmol) in dichloromethane (10 mL) was cooled to 0° C. and treated with BBr₃ (7.21 mL; 7.21 mmol, 1 M in dichloromethane) dropwise. Stirring was continued at this temperature for 1 hour and then water (30 mL) was added. The reaction mixture was extracted with dichloromethane and the combined extracts were dried (Na₂SO₄) and evaporated to dryness. The crude product was purified by flash chromatography (ether/hexane, 60:40) thereby affording the hydroxy ester 45 as a white solid (140 mg).

¹H NMR (CDCl₃) δ 3.98 (3H, s), 7.21 (1H, dd, J=8.8, 2.6 Hz), 7.26 (1H, br s), 7.79 (1H, d, J=8.8 Hz), 7.81 (1H, d, J=8.6 Hz), 7.91 (1H, dd, J=8.6, 1.7 Hz), 8.45 (1H, br s); νₘₐₓ 3500-3200 br s, 1722 m, 1693 s, 1606 s, 1274 s, 1213 s, 1129 m, 1103 m cm⁻¹.

### Example 19

### Methyl 7-hydroxy-8-nitro-2-naphthoate ester (46)

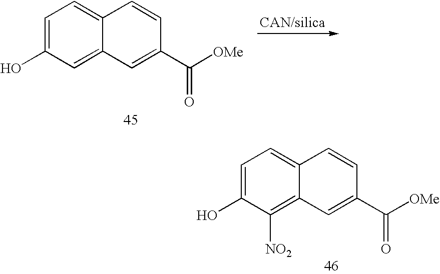

The nitro group was introduced according to a related procedure (27). The hydroxy ester 45 (140 mg; 0.69 mmol) and ceric ammonium nitrate (0.42 g; 0.77 mmol) were separately dissolved in acetonitrile (0.56 mL each) and these solutions were individually mixed to form a slurry with silica gel (0.28 g and 0.70 g respectively). Both slurries were dried under reduced pressure with vigorous stirring for more than 2 hours. Once dry both were combined in a conical flask and stirred vigorously for 40 minutes. The mixture was then applied to a prepacked column of silica (benzene/hexane, 10:90) using a glass rod to remove air bubbles from the top of the column. The column was eluted with the following solvents: benzene/hexane (10:90, 200 mL), benzene/hexane (30:70, 200 mL), benzene/hexane (40:60, 200 mL), benzene/hexane (60:40, 100 mL), benzene (100 mL), ether/hexane (10:90, 100 mL). The 8-nitro derivative 46 was obtained as a yellow solid (50 mg).

¹H NMR (CDCl₃) δ 4.02 (3H, s), 7.38 (1H, d, J=9.1 Hz), 7.88 (1H, d, J=8.4 Hz), 8.05 (1H, d, J=9.1 Hz), 8.12 (1H, dd, J=8.4, 1.6 Hz), 9.62 (1H, s), 12.08 (1H, s, OH).

### Example 20

### Methyl 6-hydroxy-5-nitro-2-naphthoate ester (47)

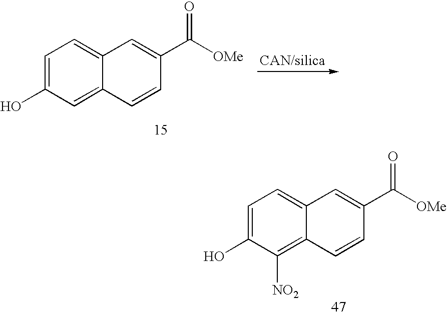

The hydroxy ester 15 (1.5 g; 7.42 mmol) in acetonitrile (6 mL) and ceric ammonium nitrate (4.47 g; 8.16 mmol) in acetonitrile (6 mL) were each slurried with silica (3 g and 7.5 g respectively). The slurries were dried under reduced pressure over ca. 2 hours and then combined in a conical flask. The mixture was stirred vigorously for 60 minutes and applied to a silica column as described above. Gradient elution of the column with benzene/hexane (10:90), benzene/hexane (50:50), ether/hexane (10:90), ether/hexane (50:50) and methanol afforded the 5-nitro derivative 47 as a yellow solid (0.94 g);

¹H NMR (CDCl₃) δ 3.99 (3H, s), 7.33 (1H, d, J=9.1 Hz), 8.10 (1H, d, J=9.1 Hz), 8.30 (1H, dd, J=9.2, 1.9 Hz), 8.53 (1H, d, J=1.8 Hz), 8.96 (1H, d, J=9.2 Hz), 12.20 (1H, br s, OH);

¹³C NMR δ 52.59, 120.60, 123.62, 127.35, 128.13, 129.66, 130.59, 131.71, 139.96, 160.07, 166.34 (C═O) (note: one 4° aromatic carbon obscured); νₘₐₓ 3500-3100 br vs, 1683 s, 1527 s, 1304 vs, 1288 s, 1203 s, 1151 m, 1110 m cm⁻¹.

### Example 21

### Methyl 6-methoxy-5-nitro-2-napthoate ester (48)

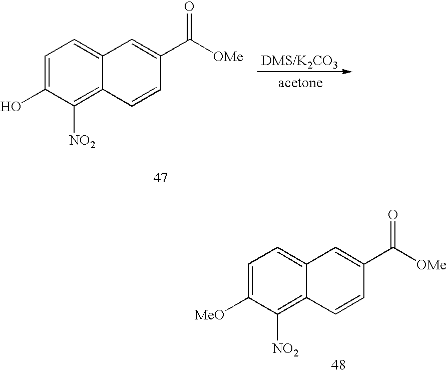

The nitro compound 47 (1.0 g; 4.05 mmol) in acetone (40 mL) was heated under reflux in the presence of K₂CO₃ (2.10 g; 16.2 mmol) and dimethyl sulfate (0.92 mL; 9.7 mmol) for 3 hours. Saturated ammonium chloride (40 mL) was added and then the aqueous layer was extracted with dichloromethane (3×40 mL). The combine extracts were washed with ammonia solution (25%, 30 mL) and dried (Na₂SO₄). Evaporation of the solvent afforded the crude product which was triturated with hexane/ether added dropwise to give the methyl ether 48 as an off-white solid (1.25 g).

### Example 22

### Methyl 5-amino-6-methoxy-2-naphthoate ester (49)

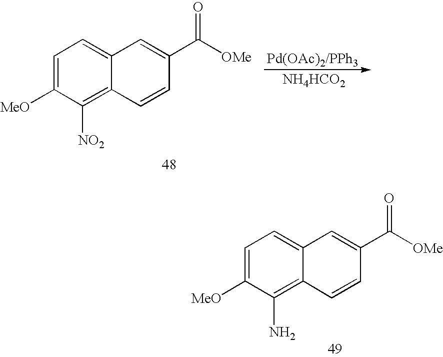

The amine 49 was prepared according to a literature procedure (28). A mixture of the nitro compound 48 (500 mg; 1.91 mmol) and 10% Pd—C (125 mg) in dry degassed methanol (10 mL) under argon was treated with anhydrous ammonium formate (555 mg; 8.81 mmol) which was added in one portion. The reaction mixture was stirred at room temperature for 1.5 hours. The catalyst was removed by filtration through a celite pad, washing with methanol (6×3 mL). The filtrate was evaporated to dryness and then the residue was treated with water (10 mL) and the mixture was extracted with dichloromethane and dried (Na₂SO₄). Evaporation of the solvents left a solid that was purified by flash chromatography (ether/hexane, 80:20) thereby affording the amine 49 as a yellow solid (210 mg).

¹H NMR (CDCl₃) δ 3.96 (3H, s), 3.99 (3H, s), 4.25 (2H, br s), 7.28 (1H, d, J=8.9 Hz), 7.44 (1H, d, J=8.8 Hz), 7.78 (1H, J=8.9 Hz), 7.98 (1H, dd, J=8.9, 1.7 Hz), 8.52 (1H, d, J=1.7 Hz);

¹³C NMR δ 52.06, 56.44, 113.63, 120.22, 120.57, 124.19, 125.03, 125.55, 128.22, 129.55, 131.89, 144.44, 167.44 (C═O); νₘₐₓ 3474 s, 3380 s, 1696 s, 1617 s, 1292 s, 1278 s, 1221 s cm⁻¹.

### Example 23

### Methyl 6-methoxy-2-naphthoate ester (51)

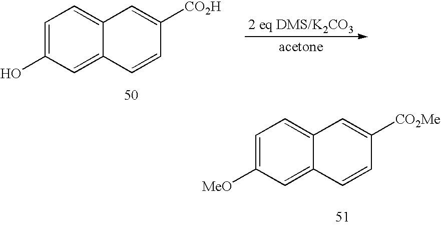

6-Hydroxy-2-napthoic acid 50 (2.0g, 0.01 mol) was dissolved in acetone (100 mL), containing potassium carbonate (6.90 g, 0.0532 mol) and then dimethyl sulfate (4.0 g; 5.40 mL; 0.032 mol) was added, dropwise. The reaction mixture was heated to reflux under nitrogen for 2.5 hours during which time all of the starting material was consumed. The reaction mixture was cooled, and then ammonium chloride (4%; 50 mL) was added. The aqueous layer was extracted with dichloromethane (3×40 mL) and the combined organic extracts washed with ammonia solution (25%, 40 mL) and dried (Na₂SO₄). Evaporation of the solvent gave the methoxy methyl ester 51. The crude product was triturated with 5% ethyl acetate/n-pentane and dichloromethane dropwise, to give a white solid (2.1 g).

### Example 24

### 2-hydroxymethyl-6-methoxynaphthalene (52)

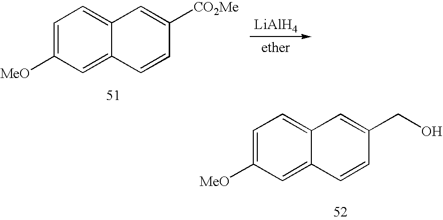

The methoxy methyl ester 51 (3.14 g; 14.5 mmol) was dissolved in dry ether (100 mL) and treated with LiAlH₄ (14.5 mL; 14.5 mmol; 1 M in THF) dropwise while cooling in ice. On completion of the addition the reaction mixture was warmed to room temperature and stirring was continued for a further 50 minutes. The reaction mixture was then cooled in ice and treated sequentially with ethyl acetate (5 mL), water (5 mL) and excess sodium sulfate until a dry solid was formed. The solid was filtered off and washed with dichloromethane. The filtrate was evaporated to dryness to give the alcohol 52 as a pale pink crystalline solid (1.95 g) after drying under high vacuum.

### Example 25

### 2-bromomethyl-6-methoxy-naphthalene (53)

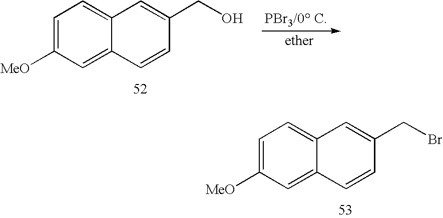

The bromide 53 was prepared according to a literature procedure (29). The alcohol 52 (1.95 g; 10.4 mmol) was partially dissolved in dry ether (150 mL) and cooled in an ice/salt/water bath. A solution of PBr₃ (1.13 mL; 11.9 mmol) in ether (20 mL) was added slowly to the stirred solution of 52 to give a white suspension. The reaction mixture was stirred with slow warming to room temperature over 2 hours at which point all solids went into solution. The resulting solution was cooled in ice and treated with 5% NaHCO₃. The ether layer was separated and washed with more 5% NaHCO₃ and dried (Na₂SO₄). Removal of the solvent left the bromide 53 as a white crystalline solid (2.05 g).

### Example 26

### 2-cyanomethyl-6-methoxy-naphthalene (54)

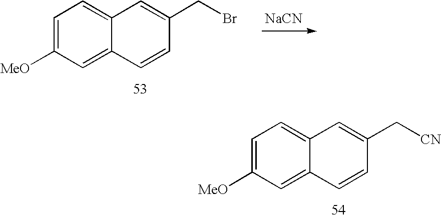

The bromide 53 (2.05 g; 8.2 mmol) was dissolved in dichloromethane (30 mL) and treated with tetrabutylammonium bromide (0.53 g; 1.63 mmol) and then a solution of sodium cyanide (1.20 g; 24.5 mmol) in water (12 mL). The reaction mixture was stirred at 50° C. for 29 hours and then diluted with ether (150 mL). The organic layer was washed with brine and dried (Na₂SO₄). Evaporation of the solvent left a solid (1.61 g) that was recrystallised from ethanol. The nitrile 54 was obtained as plates (1.19 g). The aqueous layer was treated with one volume of 1 M NaOH and 2 volumes of calcium hyochlorite overnight followed by neutralisation to decompose excess NaCN.

### Example 27

### 2-(1-cyano-1-hex-5-enyl)-6-methoxy-naphthalene (55)

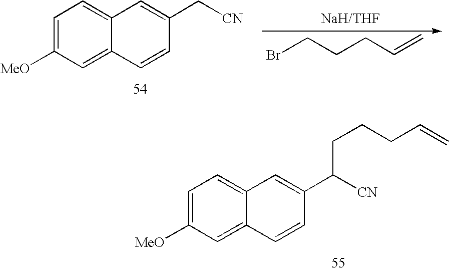

Sodium hydride (0.207 g; 8.62 mmol; 60% dispersion in oil) was added in one portion to an ice cold stirred solution of the nitrile 54 (0.85 g; 4.31 mmol) in dry DMF (10 mL). This gave rise to an intense red precipitate. After 30 minutes 4-pentenyl bromide (0.77g; 5.17 mmol; 0.61 mL) was added dropwise causing the intensity of the red colour to diminish. The reaction mixture was stirred overnight with slow warming to room temperature and after 15 hours the clear red-orange solution was poured onto ethyl acetate (100 mL) and water (50 mL) and shaken in a separating funnel. The yellow organic layer was washed with brine and dried (Na₂SO₄). Evaporation of the solvent left a yellow oil that was fractionated by flash chromatography (ether/hexane, 10:90) to give the monoalkenylated nitrile 55 as the third fraction and as a clear oil (270 mg).

### Example 28

### 2-(6-methoxy-2-naphthyl)hept-6-enoic acid (56)

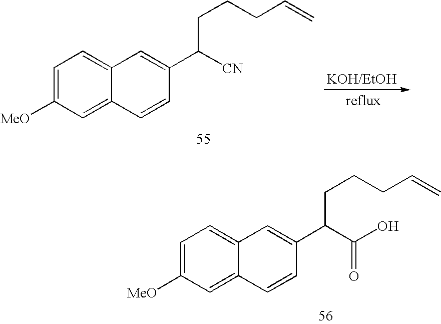

The nitrile 55 (258 mg; 0.97 mmol) was dissolved in saturated KOH in ethanol (2 mL) and allowed to stand overnight for 16.hours thereby forming a thick solid. Water (0.43 mL) was added then the whole was heated under reflux for 3 hours. The reaction mixture was cooled, diluted with water (10 mL) and extracted with ether (5 mL) to remove sideproducts and a small amount of unreacted starting material. The aqueous layer was acidified causing the required acid to precipitate. The acid was extracted with ether, the extracts were dried (Na₂SO₄) and the solvent was removed. The acid 56 was thereby obtained as a crystalline solid (67 mg).

### Example 29

### Methyl 2-(6-methoxy-2-naphthyl)hept-6-enoate ester (57)

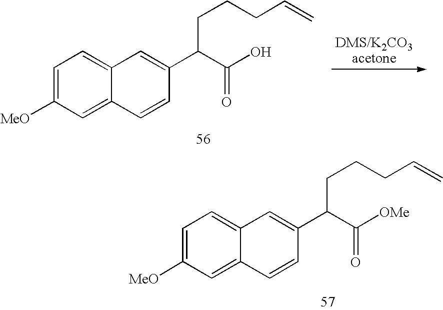

The acid 56 (67 mg; 0.24 mmol) was dissolved in acetone (5 mL) and treated with potassium carbonate (49 mg; 0.35 mmol) and dimethyl sulfate (32.8 mg; 0.26 mmol; 24.6 μL). The mixture was heated under reflux for 3 hours, cooled, diluted with 25% ammonia solution and extracted with ether. The combined extracts were dried (Na₂SO₄) and evaporated to dryness to give methyl ester 57 (66 mg).

### Example 30

### 7-hydroxy-2-(6-methoxy-2-naphthyl)heptanoic acid (58)

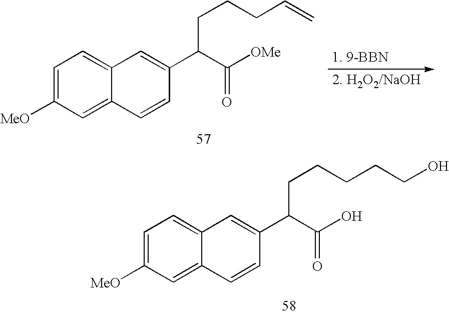

The methyl ester 57 (66 mg; 0.22 mmol) was dissolved in dry TBF(1.5 mL) and treated dropwise with 9-BBN (0.48 mL; 0.24 mmol; 0.5 M in THF) at room temperature. The reaction mixture was stirred for 3 hours and then treated sequentially with ethanol (1 mL), 6 M NaOH (0.3 mL) and then 30% H₂O₂ (0.6 mL). The whole was heated at 50° C. for 1.5 hours and then kept in the refrigerator overnight. The reaction mixture was acidified and extracted into ether. The ether extracts were dried (Na₂SO₄) and evaporated to dryness. The product mixture was fractionated by flash chromatography (ether, then MeOH/CH₂Cl—₂; 5:95-10:90) to give the hydroxyacid 58 as the most polar fraction and as a white solid (16.2 mg).

¹H NMR (CDCl₃/d₄-MeOH, 5:1) δ 1.25-2.24 (8H, m), 3.54 (2H, t, J=6.5 Hz), 3.65 (1H, t, J=7.6 Hz), 3.91 (3H, s), 7.12-7.15 (2H, m), 7.43 (1H, dd, J=8.5, 1.3 Hz), 7.68 (1H, s), 7.70 (1H, d, J=9.1 Hz);

¹³C NMR δ 25.30, 27.15, 32.02, 35.76, 51.52, 55.06, 61.99, 105.43, 118.59, 126.28, 126.37, 126.84, 128.75, 129.03, 133.52, 134.53, 157.33, 176.91 (C═O); νₘₐₓ 3500-3200 br s, 1699 s, 1605 s, 1267 s, 1028 s, cm⁻¹.

### Example 31

### Methyl 6-methoxy-8-methyl-2-napthoate ester (65)

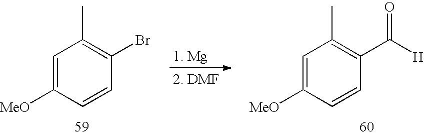

(a) The aldehyde was prepared according to a literature procedure (30). To a stirred solution of 4-bromo-3-methylanisole 59 10.0 g; 49.7 mmol; 7.02 mL) in dry THF (130 mL) in a flame-dried round-bottomed flask under nitrogen was added magnesium turnings (4.84 g; 199 mmol) and iodine (4.04 g; 15.9 mmol). The reaction mixture was heated under reflux for 4 hours before cooling to 0° C. The cloudy white solution was treated with DMF (15.4 mL; 199 mmol) and stirring was continued at 0° C. for a further 1.5 hours before warming to room temperature. The reaction was quenched with saturated NH₄Cl and the product was extracted into ether. The combined extracts were dried (MgSO₄) and evaporated to dryness to give 4-methoxy-2-methylbenzaldehyde 60 as a yellow oil (7.23 g).

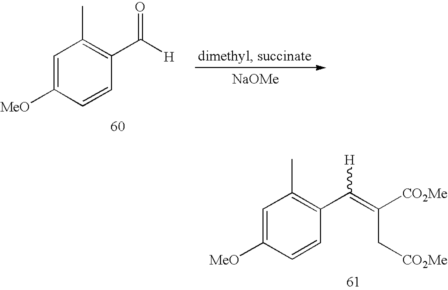

(b) Further steps (steps b-d) were carried out with some modification of a related procedure (31). To a stirred solution of the aldehyde 60 (1.50 g; 10.0 mmol) and dimethyl succinate (1.49 mL; 11.4 mmol) in methanol (26 mL) was added a solution of sodium methoxide (3.3 mL; 10.5 mmol; 3.2 M in methanol). The reaction mixture was heated under reflux for 2 hours before cooling to room temperature. The reaction volume was reduced by half under reduced pressure and the remaining solution was cooled in ice and acidified with 6 M HCl and then diluted with water (100 mL). The product was extracted into chloroform (200 mL) and the extract was dried (MgSO₄) and evaporated to give an orange oil. Flash chromatography (ethyl acetate/hexane, 25:75) afforded the monoester as a viscous oil (419 mg). This was dissolved in acetone (15 mL) and treated with anhydrous K₂CO₃ (543 mg; 3.93 mmol) and dimethyl sulfate (373 μL; 3.93 mmol). The whole was heated under reflux for 2.5 hours before cooling to room temperature and quenching with saturated ammonium chloride solution. The product was extracted into dichloromethane (3×50 mL) and the combined extracts were washed with 25% ammonia solution and dried (MgSO₄). Evaporation of the solvent gave the diester 61 as a yellow oil (485 mg). This was used in the next step without purification.

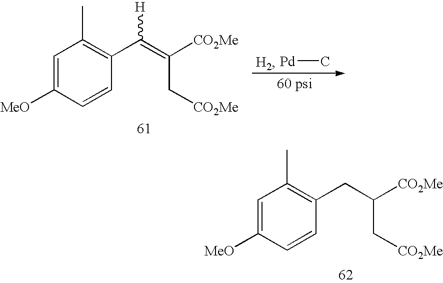

(c) The diester 61 (321 mg; 1.15 mmol) was dissolved in ethyl acetate and hydrogenated in the presence of 10% Pd—C (75 mg) on a Parr medium pressure hydrogenator at 60 psi for 20 hours. The reaction mixture was filtered through Celite and evaporated to dryness to give the saturated diester 62 as a pale yellow oil (177 mg). This was used without further purification in the next step.

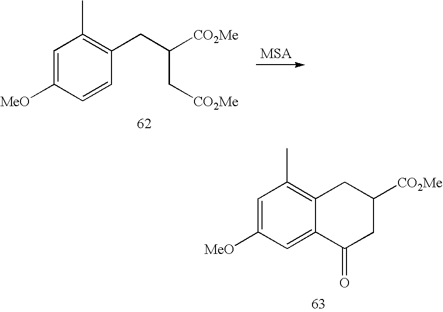

(d) The saturated diester 62 (155 mg; 0.55 mmol) in methanesulfonic acid (10 mL) was heated under reflux for 2 hours. The reaction was quenched by pouring onto ice/water (50 mL) and the product was extracted into chloroform (100 mL). The extract was dried (MgSO₄) and evaporated to dryness to give a mixture (146 mg) of the keto ester 63 and the keto acid. This mixture was treated with dimethyl sulfate (150 μL; 1.55 mmol) and K₂CO₃ (214 mg; 1.55 mmol) in boiling acetone (6 mL) as previously described (step b). Workup gave a brown oil that was purified by flash chromatography (ethyl acetate/hexane, 50:50) thereby affording the keto ester 63 as a colourless viscous oil (144 mg).

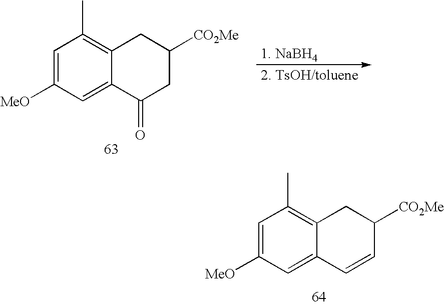

(e) The following steps (steps e and f) involve carbonyl removal and aromatisation of the A-ring. A related procedure has been reported (32). The keto ester 63 (144 mg; 0.58 mmol) was treated with sodium borohydride (20 mg) in methanol (10 mL) at 0° C. over 3 hours. The reaction was quenched with saturated ammonium chloride solution and the product was extracted with ethyl acetate. The combined extracts were washed with brine and dried (MgSO₄). Evaporation of the solvent left the hydroxy acid as a colourless oil (85 mg). This was heated under reflux in toluene (3 mL) in the presence of a few crystals of p-toluenesulfonic acid for 4 hours. After this time the reaction mixture was diluted with ethyl acetate and washed with water and brine. The organic layer was dried (MgSO₄) and evaporated to dryness to give the 1,2-dihydronaphthoate 64 as a pale yellow oil (40 mg) that was used directly in the next step without purification.

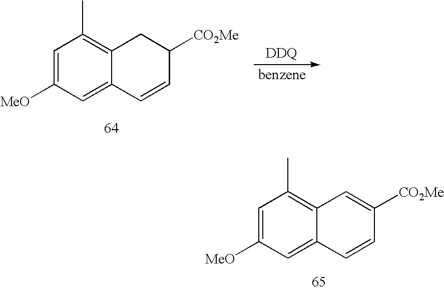

(f) The dihydronaphthoate 64 (40 mg; 0.17 mmol) was heated under reflux in benzene (4.8 mL) in the presence of DDQ (30 mg; 0.17 mmol) for 14 hours. The reaction mixture was partitioned between water and ethyl acetate and the organic layer was washed with brine and dried (MgSO₄). Evaporation of the solvent left a dark brown oil that was purified by flash chromatography (ethyl acetate/hexane, 33:67) thereby affording the naphthoate 65 as a solid (18.3 mg).

¹H NMR (d₄-MeOH) δ 2.61 (3H, 2), 3.88 (3H, s), 3.93 (3H, s), 7.00 (1H, br s), 7.05 (1H, br s), 7.72 (1H, d, J=12.9 Hz), 7.91 (1H, dd, J=12.9, 2.5 Hz), 8.56 (1H, s);

¹³C NMR δ 19.03, 52.52, 55.73, 105.12, 121.19, 126.38, 127.88, 128.64.

Biological Testing

6-Hydroxy-2-naphthalene-sulfonic acid (compound 24) was obtained commercially from Merck. Sodium-6,7-dihydroxynaphthalene-sulfonate (compound 6) was also commercially available. 6,7-dihydroxynaphthalene-2-sulfonic acid (compound 6) (cat. No. 21, 896-0), S-(+)-6-methoxy-α-methyl-2-naphthalene acetic acid (compound 8). (cat. No. 25, 478-5), 2,6-naphthalene disulfonic acid (compound 24) (cat. No. N60-5) and 6-hydroxy-2-naphthanoic acid (compound 9) (cat. No. 46, 915-7) were obtained from Aldrich.

In vitro Assay of MIF Antagonism

The activity of each compound was studied in a bioassay utilising MIF-induced proliferation of human dermal fibroblasts. S112 human dermal fibroblasts were propagated in RPMI/10% foetal calf serum (FCS). Prior to experimentation, cells were seeded at 10⁵ cells/ml in RPMI/0.1% BSA for 18 hours. At time point zero, culture medium was replaced with RPMI/10% FCS and treatments administered. Cells were treated with recombinant human macrophage migration inhibitory factor (MIF) 50 ng/ml (1.353×10⁻⁹ M) and/or the compound at a 1 or 1000 molar ratio to the concentration of MIF. In some experiments the compound was combined with MIF at time point −30 minutes, prior to adding at time point zero. At time point 30 hours, cells were pulsed with 1 μC¹⁻³H-thymidine. At time point 48 hours, cells were harvested using a semi-automated cell harvester. The radioactivity incorporated into DNA was determined by liquid scintillation counting, with results expressed as [³H] thymidine incorporation. The proliferation of untreated cells was expressed as 100% and the effect of MIF and each compound expressed in relative %.

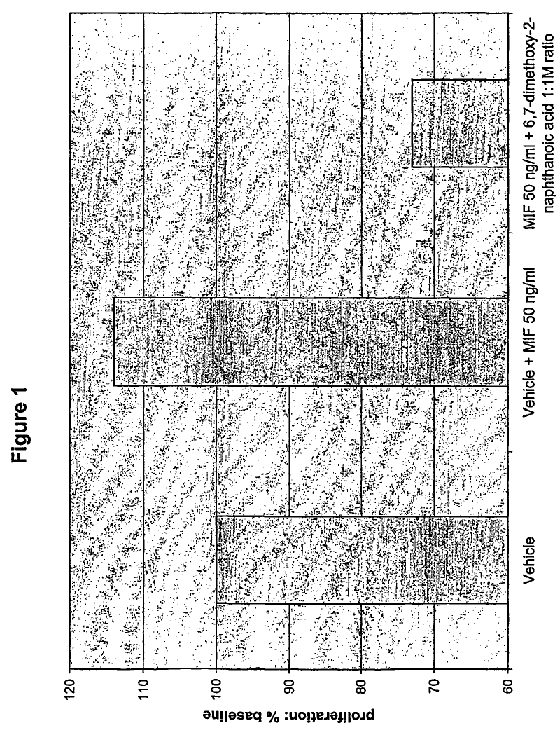

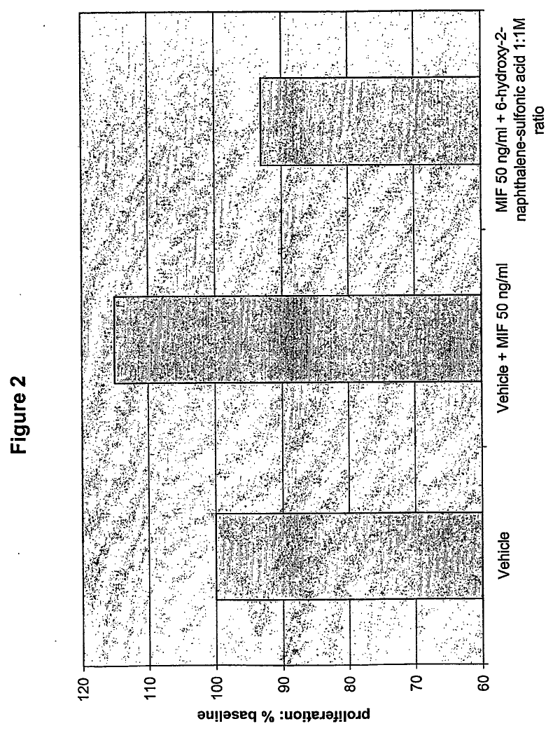

The results for 6,7-dimethoxy-2-naphthanoic acid 4 and 6-hydroxy-2-naphthalene-sulfonic acid 5 are depicted on FIGS. 1 and 2 respectively. The inhibition of MIF-induced proliferation by these compounds is consistent with their acting as inhibitors of the cytokine or biological activity of MIF.

Alternative in vitro Assay of MIF Antagonism

The activity of each compound was studied in a bioassay utilising MIF-dependent activation of human dermal fibroblasts. Sampey et al have shown that induction of the expression of cyclooxygenase-2 (COX-2) by the cytokine interleukin 1 (IL-1) is dependent upon the presence of MIF, i.e. can be prevented using specific anti-MIF monoclonal antibody (33). IL-1-induced COX-2 expression is therefore a MIF-dependent event.

S112 human dermal fibroblasts were propagated in RPMI/10% foetal calf serum (FCS). Prior to experimentation, cells were seeded at 10⁵ cells/ml in RPMI/0.1% BSA for 18 hours. Cells were treated with recombinant human IL-1 (0.1 ng/ml) and with each compound at 1-100 μM. After 6 hours, cells were collected and intracellular COX-2 protein determined by permeabilisation flow cytometry. Cells permeabilised with 0.1% saponin were sequentially labelled with a mouse anti-human COX-2 monoclonal antibody and with sheep-anti-mouse F(ab)2 fragment labelled with fluoroscein isothiocyanate. Cellular fluorescence was determined using a flow cytometer. At least 5000 events were counted for each reading, each of which was performed in duplicate, and the results expressed in mean fluorescence intensity (MFI) after subtraction of negative control-labelled cell fluorescence.

The effect of each compound was determined by subtracting the IL-1+compound-treated cell MFI from the IL-1-treated cell MFI and expressed as % inhibition.

Results are shown in Table 1, below. In each case the % inhibition of IL-1-induced COX2 expression is shown as the mean, or mean±SEM where results are available from multiple experiments.

The results show that these compounds generally exert a powerful inhibitory effect on IL-1-induced COX2 expression, consistent with a significant MIF-inhibitory effect.

TABLE 1

| | | | | number |
|---|---|---|---|---|
| Compound | Effect | Concentration (μM) | | of expts. |
| 65 | 50.40% | 50 | uM | 1 |
| 7 | 32.70% | 1 | uM | 1 |
| 10 | 27.70% | 50 | uM | 1 |
| 6 | 24.4 +/− 6.4% | 100 | uM | 2 |
| 6 | 25.7 +/− 3.6% | 50 | uM | 11 |
| 6 | 21.30% | 25 | uM | 1 |
| 6 | 22.4 +/− 5.4% | 10 | uM | 5 |
| 6 | 16.4 +/− 3.2% | 1 | uM | 5 |
| 14 | 24.1 +/− 17.1% | 50 | uM | 2 |
| 43 | 24.10% | 50 | uM | 1 |
| 15 | 17.30% | 1 | uM | 1 |
| 11 | 13.60% | 25 | uM | 1 |
| 39 | 13.30% | 50 | uM | 1 |
| 2 | 11.60% | 0.1 | uM | 1 |
| 22 | 11.60% | 50 | uM | 1 |
| 44 | 9.80% | 50 | uM | 1 |
| 46 | 9.30% | 50 | uM | 1 |
| 40 | 8.50% | 50 | uM | 1 |
| 24 | 8.20% | 1 | uM | 1 |
| 23 | 8.0 +/− 6.8% | 10 | uM | 2 |
| 28 | 7.80% | 25 | uM | 1 |
| 19 | 7.70% | 10 | uM | 1 |
| 9 | 7.50% | 1 | uM | 1 |
| 33 | 5.30% | 50 | uM | 1 |
| 4 | 2.7% | 1 | uM | 1 |

FIG. 3 shows a dose response curve for 6,7-dihydroxynaphthalene-2-sulphonic acid (compound 6). This compound was tested for IL-1 induced COX-2 expression inhibition, as discussed above at a concentration of 0.01, 0.1, 1.0, 10 and 50 μM. Dose-dependent inhibition of IL1-induced COX-2 expression was observed, consistent with compound 6 exerting an inhibitory effect on the cytokine or biological activity of MIF.

Effect of Glucocoyicoids on MIF Antagonism

In vitro Assay of MIF Antagonism in the Presence of Glucocorticoid

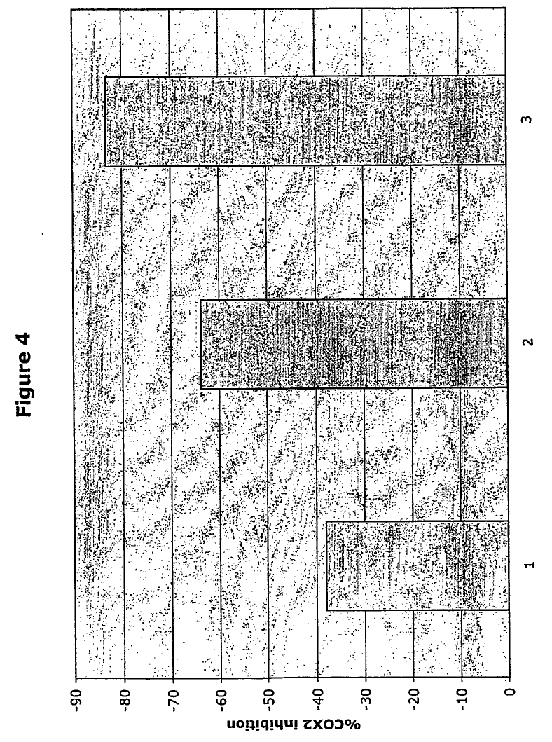

The above alternative in vitro assay for analysing IL-1 induced COX-2 expression was repeated using 6,7-dihydroxynaphthalene-2-sulfonic acid (compound 6) (6) (50 μM) (column 1), dexamethasone (10⁻⁹ M) (column 2) or a combination of dexametaasone (10⁻⁹ M) and 6,7-dihydroxynaphthalene-2-sulfonic acid (50 μM (column 3). The results are shown in Table 2 and FIG. 4. The concentration of compound 6 with dexamethasone resulted in increased effectiveness of the inhibition of IL1-induced COX-2, consistent with an effect of compound 6 on MIF cytokine or biological activity.

TABLE 2

| Experiment | Compound | % inhibition |
|---|---|---|
| 1 | 6,7-dihydroxynaphthalene-2- | 38.0 |
| | sulfonic acid | |
| 2 | dexamethasone | 63.8 |
| 3 | 6,7-dihydroxynaphthalene-2- | 83.3 |
| | sulfonic acid and dexamethasone | |

In vivo Assay of MIF Antagonism

The activity of each compound was studied in the rat adjuvant-induced arthritis (AIA) model of rheumatoid arthritis. This model has been demonstrated to be dependent on MIF (34). Male Sprague-Dawley rats (150±20 g) were used. Adjuvant artritis was induced by intradermal injection at the tail base of 150μ of a 10 mg/ml suspension of heat-inactivated Mycobacterium tuberculosis (Difco, Detroit, Mich.) in squalane. The compound was administered at a dose of 1.0 mg/kg body weight by once daily intraperitoneal injection on each day (treated). Control animals received an identical volume injection of vehicle (control). Joint inflammation in adjuvant arthritis was assessed clinically as follows:
i) Articular index/score: A score of 0 (no observable erythema or swelling) to 4 (severe swelling and erythema) was given for each paw. All four paws were scored, resulting in a maximum possible score of 16 for each animal (34).
ii) Synovial fluid cell number: Joints were exposed by removal of overlying skin, needle arthrocentesis performed and joint space cells obtained by closed needle lavage with 2 ml saline using a 26 gauge needle and syringe. Lavaged cells from both ankle joints were pooled, washed in saline (300 g for 5 minutes), and counted in a hemocytometer (Improved Nebauer, Weber, UK) (34).

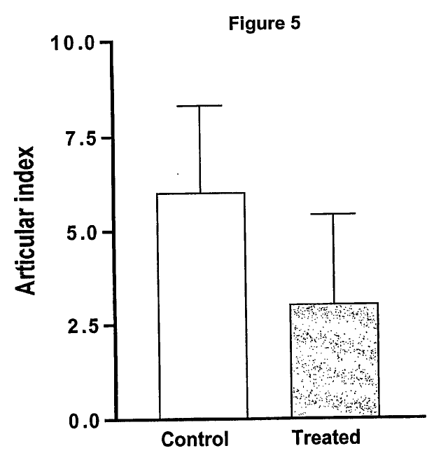

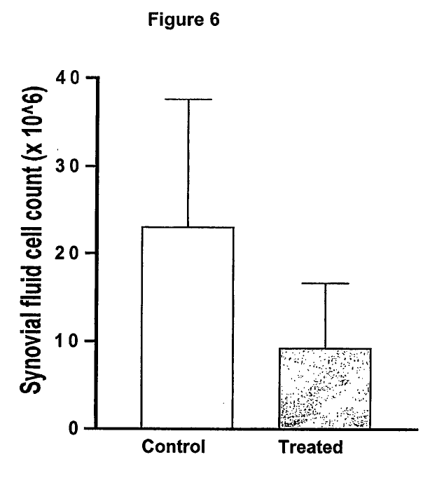

The results for 6,7-dimethoxy-2-naphthanoic acid (compound 2) in relation to i) and ii) are depicted in FIGS. 5 and 6 respectively. Compound 2 administration resulted in a significant inhibition of arthritis severity, consistent with an inhibitory effect on MIF cytokine or biological activity.

Alternative in vivo Assay of MIF Antagonism

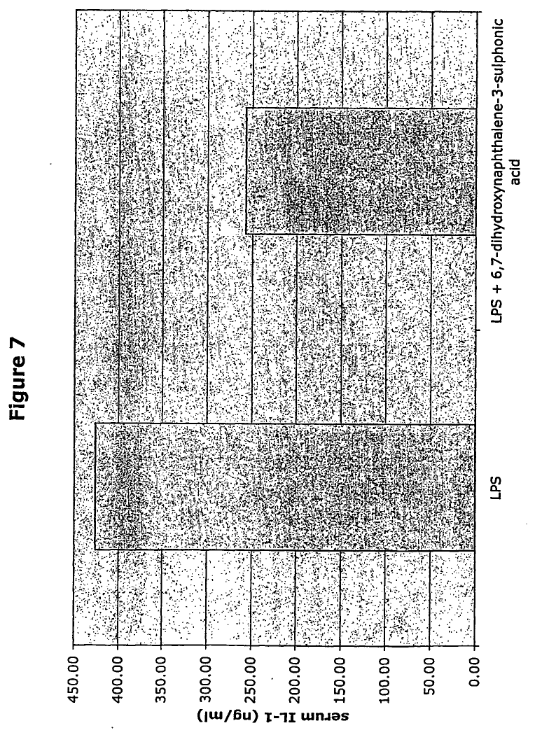

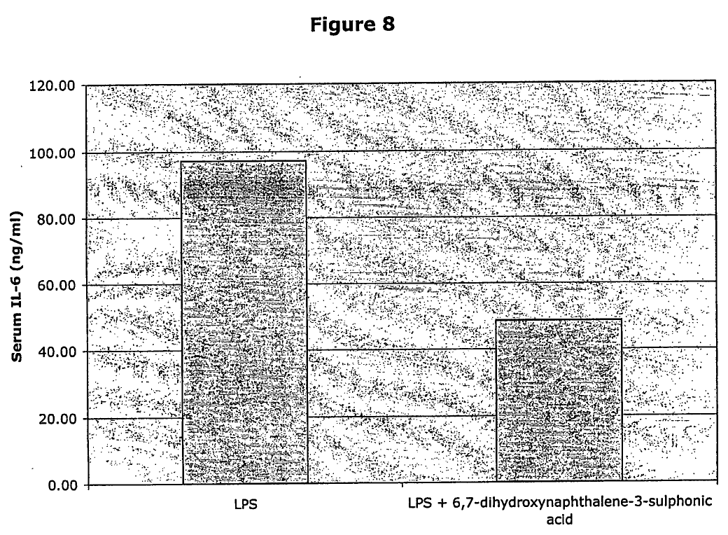

The activity of 6,7-dihydroxynaphthalene-3-sulfonic acid (compound 6) was studied in the murine endotoxic shock model. This model has been previously shown to be dependent on MIF (35). Reductions in the toxic effects of endotoxin were observed in animals treated with anti-MIF antibodies (35). A substance capable of exerting an inhibitory effect on the cytokine or biological effect of MIF may be expected to result in reductions in the serum concentration of cytokines such as interleukin 1 or interleukin 6. Endotoxaemia was induced by intra-peritoneal injection of lipopolysaccharide (LPS) (15 mg/kg) in 400 μl saline. Mice were treated with a saline solution (control) only, a saline solution and LPS, or LPS and 6,7-dihydroxynaphthalene-2-sulfonic acid (compound 6) at a dose of 15 mg/kg body weight by intra-peritoneal injection at 24 hours, 12 hours and 1 hour before intra-peritoneal LPS injection. After 1.5 or 6 hours mice were humanely killed by CO₂ inhalation then neck dislocation. Serum was obtained from blood obtained by cardiac puncture prior to death and measured for cytokines including interleukin 1 (IL-1) and interleukin 6 (IL-6) by ELISA. The production of IL-1 and IL-6 has been previously shown to be dependent on MIF (36). FIG. 7 shows analysis of serum IL-1 (ng/ml) when LPS is administered alone or in combination with 6,7-dihydroxynaphthalene-2-sulfonic acid. FIG. 8 shows analysis of serum IL-6 (ng/ml) when LPS is administered alone or in combination with 6,7-dihydroxynaphthalene-2-sulfonic acid (compound 6).

The effect of compounds was further tested under a variety of conditions in animals exposed to endotoxic shock induced as above by the injection of 15 mg/kg LPS by intraperitoneal injection. In each case, compounds were administered by intraperitoneal injection at a dose of 15 mg/kg. Compound administration was associated with reductions in serum cytokine concentration under a variety of administration regimens. These data, shown in Table 3, suggests that compounds of formula (I) are active inhibitors of the biological or cytokine activity of MIF.

TABLE 3

| | Treatment | | | | LPS plus | |
|---|---|---|---|---|---|---|
| Compound | regimen* | Result | control | LPS | compound | Inhibitory |
| 23 | −16 h | serum IL-1 | 12 ± 5 | 105 ± 29 | 58 ± 13 | Y |
| | | (ng/ml) | | | | |
| 6 | −18 h | Serum TNF | 4263 ± 1399 | 6664 ± 1124 | 3970 ± 2565 | Y |
| | | (pg/ml) | | | | |
| 24 | −24 h, −1 h | Serum IL-6 | | 776 ± 499 | 144 ± 18 | Y |
| | | (ng/ml) | | | | |
| 6 | −24 h, −1 h | Serum IL-6 | | 199 ± 41 | 150 ± 19 | Y |
| | | (ng/ml) | | | | |
| 6 | −24 h, −12 h, | Serum IL-1 | | 484 ± 87 | 257 ± 70 | Y |
| | −1 h | (ng/ml) | | | | |
| 6 | −24 h, −1 h | Serum IL-6 | | 142 ± 6 | 104 ± 11 | Y |
| | | (ng/ml) | | | | |

*times refer to the time points prior to administration of LPS at which compound was administered. All treatments were administred by intra-peritoneal injection.

In vitro Toxicity Assay

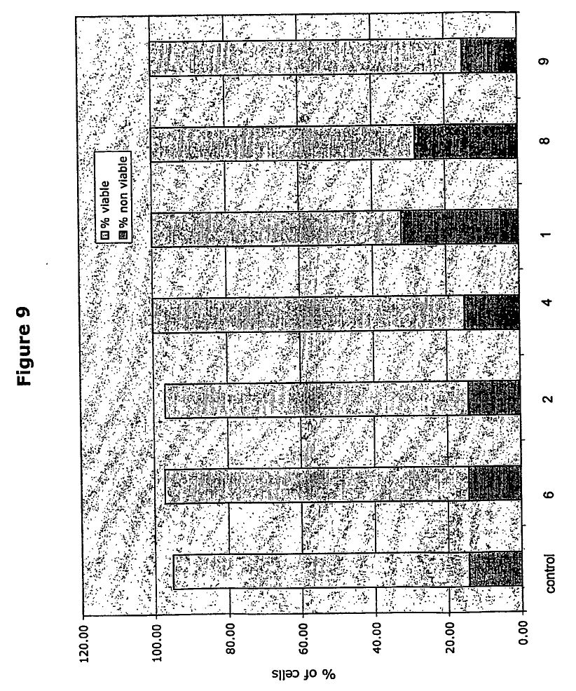

The compounds of formula (I) may have low toxicity towards cells. The toxicity of compounds of formula (I) were examined in vitro to assess cytotoxicity. Human dermal fibroblast cell line (S112) cells were exposed to vehicle (control) or compounds of formula (I) (50 μM) in vehicle. Toxicity was assessed by analysis of apoptosis using flow cytometric detection of cell surface Annexin V binding and propidium iodide staining. At least 5000 events were analysed for each experiment. Cells positive for both Annexin V and propidium iodide were designated as apoptotic and cells negative for both Annexin V and propidium iodide were designated as viable. Results are expressed as the percentage (%) of cells with each of these labels. No compound of formula (I) induced apoptosis at levels above the control. The results for a number of compounds of formula (I) are shown in FIG. 9.

TABLE 4
Key to compounds tested in FIG. 9

| Compound | Name |
|---|---|
| 6 | 6,7-dihydroxynaphthalene-2-sulphonic acid |
| 2 | 6,7-dimethoxynaphthalene |
| 4 | 6,7-dimethoxy-2-naphthanoic acid |
| 1 | 6,7-dihydroxynaphthalene |
| 8 | (S)-(+)-6-methoxy-α-methyl-2-naphthalene acetic acid |
| 9 | 6-hydroxy-2-naphthanoic acid |

In vitro Assay of MIF Antagonism: T Cell Activation

Activation of T lymphocyte responses is a critical event in the development of autoimmune and chronic inflammatory diseases. T lymphocyte activation in vitro and in vivo are known to be dependent upon the presence of bioactive MIF. For example, administration of specific monoclonal antibodies directed against MIF have been shown to inhibit development of T cell activation in vitro and of cutaneous delayed-type hypersensitivity responses in vivo (37) (7). The demonstration that compounds inhibitory of the cytokine and biological activity of MIF are inhibitory of T cell activation in vitro will be seen by those skilled in the art as supportive of the biological and functional antagonism of MIF provided by those compounds.

C57BL6/J male mice, aged 7-10 weeks old, were immunised with 200 μg of methylated bovine serum albumin (MBSA) dissolved in 20 μL of saline, emulsified in 200 μL of Freund's complete adjuvant (FCA) by subcutaneous injection. Seven (7) days later mice received a booster immunisation with 100 μg mBSA in 10 μL saline plus 100 μL FCA by subcutaneous injection. After a further seven (7) days mice were killed and spleens collected aseptically into Hanks buffered saline solution (HBSS). A single cell suspension was prepared in Petri dishes by flushing DMEM through the organ using a 26G needle and 2 mL syringe. The resulting cell suspension was centrifuged for 5-7 minutes and supernatant discarded. Erythrocytes were lysed using a solution containing 0.579% NH₄Cl, 0.000037% EDTA, and 0.1% NaHCO₃ in a 37° C. water bath. Tubes were then filled with DMEM and centrifuged for 5-7 minutes. The cell-containing pellet was then resuspended in DMEM containing 5% fetal calf serum (FCS) and 0.05% 2-mercapto-ethanol at a concentration of 1×10⁶ cells/mL and plated at 1×10⁵ cells/well in 96-well plastic tissue culture plates. Test substances (compound or vehicle) were added and incubated for 1 hour in a 37° C., 5% CO₂ incubator. The specific stimulating antigen, mBSA, was then added at 10-50 μg/mL and plates incubated for 30 hours in a 37° C., 5% CO₂ incubator. Tritiated³H-thymidine was then added at a concentration of 0.5 μCi/well for a further 18 hours. Cells were harvested on a Packard cell harvester, and the harvested material added to 750 μL/tube scintillation fluid. Scintillation counts were read on a Wallac beta-emnission counter. Incorporation of ³H-thymidine into DNA is a measure of cell proliferation, and hence of antigen-specific T cell activation.

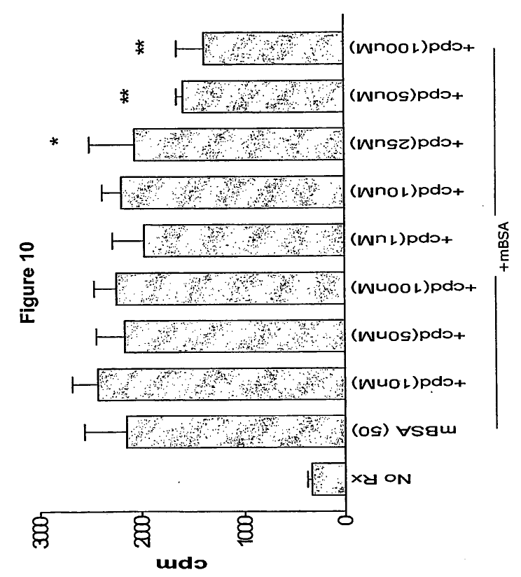

As shown in FIG. 10, T cell proliferation was significant increased in the presence of the specific sensitising antigen, mBSA, at 50 μg/mL. The addition of compound 23 in increasing concentrations exerted a dose-dependent and statistically significant inhibitory effect on antigen-specific T cell activation. In FIG. 10, asterisks signify a statistically significant result (*p<0.05, **p<0.01).

The concentration at which T cell activation was suppressed by 50% compared to vehicle-only-treated cells (EC50) was calculated using Prism® software.

Further compounds were also tested for their ability to inhibit antigen-specific T cell activation as a marker of the inhibition of the cytokine or biological activity of MIF using this assay. Table 5 lists the EC50 for each compound in this assay, performed with concentrations of mBSA of either 50 or 10 μg/ml.

TABLE 5

| | mBSA 50 μg/ml | | mBSA 10 μg/ml | |
|---|---|---|---|---|
| Compound | EC50(μM) | no. expts | EC50(μM) | no. expts |
| 14 | 0.12 | 1 | 10.20 | 1 |
| 35 | 0.22 | 1 | 8.89 | 1 |
| 33 | 0.54 | 1 | 12.50 | 1 |
| 19 | 0.95 | 1 | 10.84 | 1 |
| 28 | 1.18 | 1 | 6.40 | 1 |
| 11 | 3.34 | 1 | 0.02 | 1 |
| 40 | 8.90 | 1 | 2.57 | 1 |
| 49 | 11.91 | 1 | 1.01 | 1 |
| 10 | 16.67 | 1 | 10 | 1 |
| 65 | 17.97 | 1 | Not done | |
| 15 | 21.47 | 3 | 51.74 | 1 |
| 58 | 22.26 | 1 | Not done | |
| 46 | 28.07 | 1 | Not done | |
| 45 | 32.35 | 1 | 14.76 | 1 |
| 43 | 41.49 | 1 | 1.85 | 1 |
| 4 | 49.54 | 3 | 14.84 | 2 |
| 2 | 51.97 | 3 | 18.14 | 1 |
| 9 | 87.71 | 3 | 72.24 | 1 |
| 39 | 89.77 | 1 | 49.27 | 1 |
| 6 | 104.40 | 4 | 21.76 | 2 |
| 23 | >100 | 3 | >100 | 1 |
| 5 | >100 | 3 | 2.39 | 1 |
| 8 | >100 | 3 | 57.67 | 1 |
| 22 | >100 | 1 | 3.5 | 1 |
| 44 | >100 | 1 | 12.72 | 1 |
| 47 | >100 | 1 | 13.13 | 1 |

In vivo Assay of MIF Antagonism: Antigen-Induced Arthritis.

Rheumatoid arthritis is a common, serious, chronic inflammatory disease affecting synovial joints, of which the etiology is unknown. Rheumatoid arthritis is one of the most common autoimmune or chronic inflammatory diseases, and can be seen as a model for other, less common, autoimmune and chronic inflammatory diseases. MIF has been confirmed as an important mediator in several animal models of rheumatoid arthritis, through studies in which antagonism of MIF with a monoclonal anti-MIF antibody exerted significant inhibitory effects on disease (38) (34) (8). Included among the animal models of rheumatoid arthritis in which MIF has been shown to be an essential factor is murine antigen-induced arthritis (8). A compound which inhibits the cytokine of biological activity of MIFF might be expected to inhibit the development of murine antigen-induced arthritis in vivo.

Antigen-induced arthritis was induced in mice. C57BL6/J male mice, aged 7-10 weeks old, were immunized on day 0 with 200 μg methylated BSA (mBSA) emulsified in 200 μl of Freund's complete adjuvant (FCA) injected subcutaneously into the flank skin. Mice were treated with compound 5, administered by intraperitoneal injection, once per 24 hours at a dose of 15 mg/kg body weight. After seven days, mice received 100 μg “mBSA and 100 μl FCA by intradermal injection at the base of the tail. After a further 14 days, arthritis was induced by intra-articular injection of 30 μg mBSA in 10 μl of sterile saline into the left knee, the right knee being injected with sterile saline alone.

Arthritis was analysed histologically at day 28 after first immunisation. Knee joints were dissected and fixed in 10% buffered formalin for 7 days. Fixed tissues were decalcified for 3 weeks in 15% ethylene-diamine-tetra-acetic acid (EDTA), dehydrated and embedded in paraffin. Sagittal sections (5 μm) of the knee joint were stained with Safranin-O and counterstained with fast green /iron hematoxylin. Histological sections were scored from 0 to 3 for four parameters: Synovitis was defined as hyper-cellularity of the synovium including pannus formation. Joint space exudate was identified as leukocytes, discretely or in aggregates, in the joint space. Cartilage degradation was defined as the loss of Safranin-O staining of articular cartilage (0=full stained cartilage, 3=totally unstained cartilage). Bone damage was defined as the extent and depth of the subchondral bone invasion by pannus. A total score was also generated from the sum of these four parameters (maximum 12).

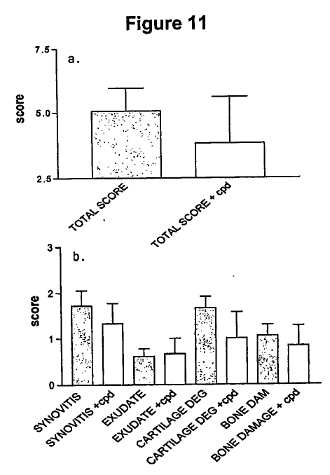

The results of treating mice with compound 23 are shown in FIG. 11. In FIG. 11a, the total arthritis score for vehicle and compound-treated animals is presented graphically. A clinically significant reduction in total arthritis score is seen. In FIG. 11b, individual parameters of arthritis are presented graphically. Clinically significant reductions in the severity of all individual parameters of arthritis can be seen for animals treated with compound 23.

In vivo Assay of MIF Antagonism: ex vivo T Cell Activation

As MIF is important in T cell activation, a compound capable of inhibiting the cytokine or biological activity of MIF might be expected to be exert inhibitory effects on T cell responsiveness. In vivo administration of such a compound might be expected to exert effects on T cell responsiveness even after the T cells have been removed from exposure to the compound, that is, if T cells were studied ex vivo after in vivo treatment with the MIF antagonist compound. To measure ex vivo antigen-specific T cell activation, spleens were removed from mice with murine antigen induced arthritis, induced as above with mBSA, at day 28 after first immunisation and a single cell suspension prepared in DMEM containing 5% FCS and 0.05% 2-mercaptoethanol. 1×10⁵ cells /200 μl were cultured in triplicate in the presence or absence of mBSA (0.1, 1.0, 10 μg /ml) in 96-well plates for 48 hours (37° C., 5% CO₂) The T cell proliferation response was determined by measuring ³H-thymidine incorporation during the final 18 hr. The cells were harvested and radioactivity incorporation into the DNA was measured with a Wallac 1409 liquid scintillation counter. The means of each triplicate culture were calculated. Each experiment comprised at least three individual animals and the results presented represent the mean±SEM of groups of animals in each experiment. The percentage inhibition of T cell proliferation was calculated using the result of the ³H-thymidine incorporation of cells from compound-treated animals divided by the ³H-thymidine incorporation of cells from vehicle-treated animals.

Table 6 displays the results obtained using splenic T cells obtained from mice which received in vivo administration of compound 4. The compound exerted an inhibitory effect on ex vivo splenic T cell proliferation.

TABLE 6

| Compound | % inhibition | [mBSA] (ug/mL) | no. expts |
|---|---|---|---|
| 4 | 18% | 10 | 1 |

In vitro Assay of MIF Antagonism: Dermal Fibroblast Proliferation Induced by Recombinant MIF.

It is well known to those skilled in the art that MIF is able to induce proliferation in a number of cell types including cells derived from patients with rheumatoid arthritis (39). It has also been demonstrated that antagonism of MIF with a monoclonal anti-MIF antibody can inhibit the proliferation of cells in vitro. A compound with the ability to inhibit the cytokine or biological function of MIF might be expected to inhibit the proliferative effect of MIF.

The activity of compound 5 was studied in a bioassay utilising MIF-induced proliferation of human dermal fibroblasts. S112 human dermal fibroblasts were propagated in RPMI/10% foetal calf serum (FCS). Prior to experimentation, cells were seeded at 10⁵ cells/ml in RPMII/0.1% BSA for 18 hours. At time point zero, culture medium was replaced with RPMI/10% FCS and treatments administered. Cells were treated with recombinant human macrophage migration inhibitory factor (MIF) 50 ng/ml and/or compound 5 at a 1-1000 molar ratio to the concentration of MIF. At a time point 30 hours later, cells were pulsed with 1 μCi/well of ³H-thymidine. At a time point 48 hours after commencement of the experiment, cells were harvested using a semi-automated cell harvester. The radioactivity incorporated into DNA was determined by liquid scintillation counting, with results expressed as [³H] thymidine incorporation.

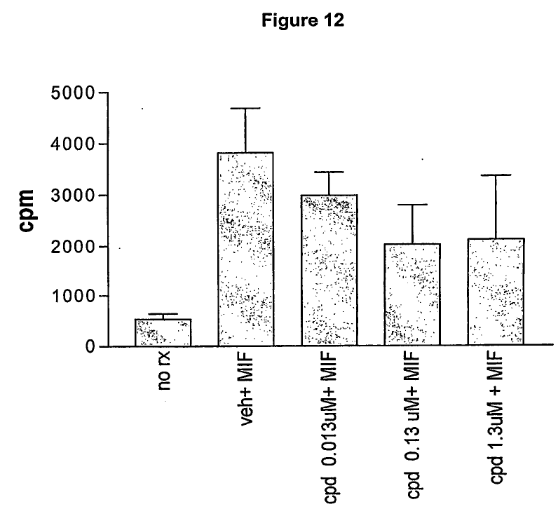

FIG. 12 depicts graphically the effect of compound 6 (0.013-1.3 μM) on proliferation of S112 cells treated with recombinant human MIF. A marked inhibitory effect was observed. The data presented are the mean±SEM of six separate experiments.

In table 7, the inhibitory effect of a number of compounds are expressed as the % inhibition of proliferation, compared to the proliferation of vehicle plus rhMIF-treated cells.

TABLE 7

| Compound | % inhibition | concentration (μM) | no. expts |
|---|---|---|---|
| 1 | 47% | 0.13 | 11 |
| 6 | 47% | 0.13 | 7 |
| 7 | 41% | 0.13 | 3 |
| 8 | 36% | 0.13 | 6 |
| 4 | 24% | 0.013 | 6 |
| 24 | 18% | 0.013 | 7 |
| 8 | 4% | 0.013 | 6 |

In vitro Assay of MIF Antagonism: Inhibition of Peritoneal Macrophage Cytokine Production.

MIF is known to be a participant in the innate immune response to toxins such as the bacterial endotoxin lipopolysaccharide (LPS). As shown above, antagonists of MIF can inhibit endotoxin-induced macrophage cytokine production in vivo. A compound with the ability to inhibit the cytokine or biological function of MIF might be expected to inhibit the activation of cytokine production by macrophages in response to LPS.

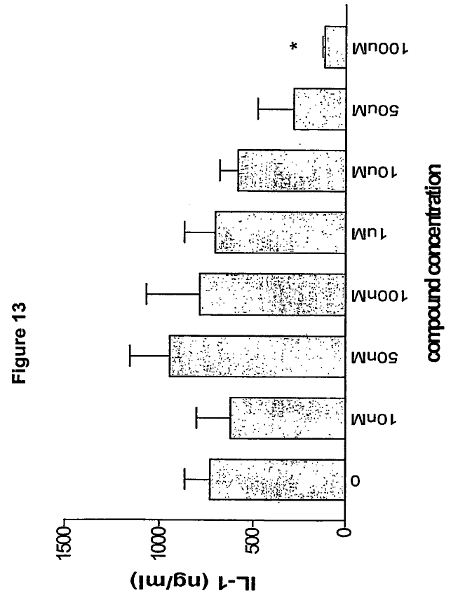

C57BL6/J male mice were injected intraperitoneally with 2ml of thioglycollate. Five (5) days later peritoneal macrophages were collected by ravaging the peritoneum of anaesthetized mice with 3 ml of cold Hanks buffered saline solution. Cells from several mice were pooled, washed and re-suspended in DMEM supplemented with 5% FCS. Cells were plated in 96 well plastic tissue culture plates at 1×10⁵ cells/well. Cells were treated with compound or vehicle for 1 hour in a 5% CO₂ incubator at 37° C. Cells were then treated with LPS (10 ng/ml) and incubated for 24 hours. After 24 hours, 50 μl of supernatant from each well was carefully removed and transferred to ELISA plates. The concentration of interleukin 1 (IL-1 ) was measured by ELISA. The concentration of compound at which LPS-induced cytokine release was suppressed by 50% compared to vehicle-only-treated cells (EC50) was calculated using Prism® software. FIG. 13 and Table 8 provide the data for compound 6 tested in this assay.

In FIG. 13, the results of a dose-response experiment with compound 6 are depicted graphically. This is representative of two independent experiments. A marked and statistically significant inhibition of macrophage IL-1 release was observed in cells treated with compound 6 (*p<0.02).

In table 8, the EC50 data for similar experiments with compound 6 are presented. These results are consistent with the inhibition of the biological and cytokine activity of MIF by compound 6.

TABLE 8

| Compound | EC50 (μM) | [LPS] ng/mL | no. expts | P value |
|---|---|---|---|---|
| 6 | 27.04 | 10 | 2 | <0.02 |

In vitro Assay of MIF Antagonism: Inhibition of Peritoneal Macrophage Nitric Oxide Release.

MIF is able to induce or facilitate the expression and release of a wide variety of pro-inflammatory and/or destructive molecules. In the case of macrophages, in addition to the facilitation of cytokine release, MIF is able to facilitate the release of nitric oxide (NO) (40). A compound with the ability to inhibit the cytokine or biological function of MIF might be expected to inhibit the activation of NO production by macrophages.

C57BL6/J male mice were injected intraperitoneally with 2ml of thioglycollate. Five (5) days later peritoneal macrophages were collected by ravaging the peritoneum of anaesthetized mice with 3 ml of cold Hanks buffered saline solution. Cells from several mice were pooled, washed and re-suspended in DMEM supplemented with 5% FCS. Cells were plated in 96 well plastic tissue culture plates at 1×10⁵ cells/well. Cells were treated with compound or vehicle for 1 hour in a 5% CO₂ incubator at 37° C. Cells were then treated with LPS (10 ng/ml) and recombinant human interferon-γ (10 units/ml) and incubated for 24 hours. After 24 hours, 50 μl of supernatant from each well was carefully removed and transferred to ELISA plates. The concentration of nitrite in culture supernatants was measured by the Greiss assay (41). The results were defined as the percentage inhibition of nitrite concentration in compound-treated cell culture supernatants compared to that of vehicle-treated cells.

Table 9 displays the results for compound 2 tested in this assay. Marked and statistically significant reductions in nitrite concentration were observed in the supernatants of cells treated with compound 2. These data are consistent with compound 2 exerting an inhibitory effect on the cytokine and biological activity of MIF.

TABLE 9
Inhibition of murine peritoneal macrophage
nitric oxide production.

| | Concentration | % Nitrite concentration | |
|---|---|---|---|
| Compound | (uM) | inhibition from control | P value |
| 2 | 25 uM | 5.8 +/− 1.6% | |
| | 50 uM | 8.5 +/− 2.1% | P < 0.01 |
| | 100 uM | 13.6 +/− 0.8% | P < 0.001 |

### REFERENCES

(1) David, J, Delayed hypersensitivity in vitro: its mediation by cell-free substances formed by lymphoid cell-antigen interaction. Proceedings of the National Academy of Sciences USA, 1966; 56:72-77.
(2) Weiser, W Y, Temple P A, Witek-Gianotti J S, Remold H G, Clark S C, Davis J R, Molecular cloning of cDNA encoding a human macrophage migration inhibitory factor, Proceedings of the National Academy of Sciences USA, 1989; 86:7522-7526.
(3) Leech M, Metz C N, Smith M, Weedon H, Holdsworth S R, Bucala R, et al. Macrophage migration inhibitory factor (MIF) in rheumatoid arthritis: Evidence for pro-inflammatory function and regulation by glucocorticoids. Arthritis & Rheumatism 1999; 42:1601-1608.
(4) Morand E F, Bucala R, Leech M. Macrophage migration inhibitory factor (MIF): An emerging therapeutic target in rheumatoid arthritis. Arthritis & Rheumatism 2003; 48:291-299.
(5) Calandra T, Bernhagen J, Metz C N, Spiegel L A, Bacher M, Donnelly T, et al. MIF as a glucocorticoid-induced modulator of cytokine production. Nature 1995; 377:68-71.
(6) Donnelly S C, Haslett C, Reid P T, Grant I S, Wallace W A H, Metz C N, I. Regulatory role for macrophage migration inhibitory factor in acute respiratory distress syndrome. Nature Medicine 1997; 3:320-323.
(7) Bacher M, Metz C N, Calandra T, Mayer K, Chesney J, Lohoff M, et al. An essential regulatory role for macrophage migration inhibitory factor in T-cell activation. Proceedings of the National Academy of Sciences USA 1996; 3:7849-7854.
(8) Santos L L, Hall P, Metz C N, Bucala R, Morand E F. Role of macrophage migration inhibitory factor (MIF) in murine antigen-induced arthritis: Interaction with glucocorticoids. Clin. Exp. Immunol. 2001; 123:309-314.
(9) Leech M, Santos L L, Metz C, Holdsworth S R, Bucala R, Morand E F. Control of macrophage migration inhibitory factor (MIF) by endogenous glucocorticoids in rat adjuvant arthritis. Arthritis & Rheumatism 2000; 43:827-833.
(10) Bucala R. MIF rediscovered: cytokine, pituitary hormone, and glucocorticoid-induced regulator of the immune response. FASEB. J. 1996; 10:1607-1613.
(11) Sabroe I, Pease J E, Williams T J. Asthma and MIF: innately Th1 and Th2. Clin Exp Allergy 2000; 30(9):1194-6.
(12) Tetrahedron, 1998 54(35), 10493-10511.
(13) Chem. Commun., 1997, 16, 1573-1574.
(14) J. Med. Chem., 1997,40, 1186-1194.
(15) Swantek J L, Cobb M H, Geppert T D. Jun N-terminal kinase/stress-activated protein kinase (JNK/SAPK) is required for lipopolysaccharide stimulation of tumor necrosis factor alpha (TNF) translation: glucocorticoids inhibit TNF translation by blocking JNK/SAPK. Molecular and Cellular Biology 1997;17:6274-6282.
(16) Rogatsky I, Logan S K, Garabedian M J. Antagonism of glucocorticoid receptor transcriptional activation by the c-Jun N-terrninal kinase. Proc. Natl. Acad. Sci. USA. 1998;95:2050-2055.
(17) Kassel O. Sancono A, Kratzschmar J. Kreft B, Stassen M, Cato A C. Glucocorticoids inhibit MAP kinase via increased expression and decreased degradation of MKP-1. Embo J 2001;20(24):7108-16.
(18) Mitchell, R A, Metz C N, Peng T, Bucala R. Sustained mitogen-activated protein kinase (MAPK) and cytoplasmic phospholipase A2 activation by macrophage migration inhibitory factor (MIF). Regulatory role in cell proliferation and glucocorticoid action. Journal of Biological Chemistry, 1999; 274:18100-18106.
(19) Cory, R. P.; Becker, R. R; Rosenbluth, R.; Isenberg, I. J. Am. Chem. Soc., 1968, 90, 1643.
(20) Bäckström, R.; Pystynen, J.; Lotta, T.; Ovaska, M.; Taskinen, J. WO 0222551.
(21) Connor, D. T.; Cetenko, W. A.; Mullican, M. D.; Sorenson, R. J.; Unangst, P. C.; Weikert, R. J.; Adolphson, R. L.; Kennedy, J. A.; Thueson, D. O.; Wright, C. D.; Conroy, M. C. J. Med. Chem., 1992, 35, 958.
(22) Newman, M. S.; Kames, H. A., J. Org. Chem., 1966, 31, 3980.
(23) Hovorka, M.; \check{S}\check{c}igel, R.; Gunterova, J.; Tichy, M.; Závada, J. Tetrahedron, 1992, 48, 9503.
(24) Wehrmeister, H. L. J. Org. Chem., 1961, 26, 3821.
(25) Saá, J. M.; Martorell, G.; García-Raso, A. J. Org. Chem., 1992, 57, 678.
(26) Cabri, W.; De Bernardinis, S.; Francalanci, F.; Penco, S. J. Org. Chem., 1990, 55, 350.
(27) Chawla, H. M; Mittal, R. S. Synthesis, 1985, 70.
(28) Ram, S.; Ehrenkaufer, R. E. Tetrahedron Lett., 1984, 25, 3415.
(29) Wang, J. -Q.; Weyand, E. H.; Harvey, R. G. J. Org. Chem., 2002, 67,6216.
(30) Tang, X.; Soloshonok, V. A.; Hruby, V. J. Tetrahedron Asymm., 2000, 11,2917.
(31) Weinstock, J.; Gaitanopoulos, D. E.; Oh, H. -J.; Pfeiffer, F. R.; Karash, C. B.; Venslavsky, J. W.; Sarau, H. M.; Flaim, K E.; Hieble, J. P.; Kaiser, C. J. Med. Chem., 1986, 29, 1615.
(32) Akpuaka, M. U.; Beddoes, R. L.; Bruce, J. M.; Fitzjohn, S.; Mills, 0. S. J. Chem. Soc., Chem. Commun., 1982, 686.
(33) Sampey, A, V, Hall P, Bucala R, Morand E F, Macrophage Migration Inhibitory Factor (MIF) activation of rheumatoid synoviocytes, Arthritis & Rheumatism; 1999; 44: S283.
(34) Leech. M, Metz C N, Santos L L, Peng T, Holdworth S R, Bucala R. et al., Involvement of Macrophage Migration Inhibitory Factor in the evolution of rat adjuvant arthritis, Arthritis & Rheumatism., 1998; 41:910-917.
(35) Bernhagen, J, Calandra T, Mitchell R A, Martin S B, Tracey K J, Voelter W, et al. MIF is a pituitary-derived cytokine that potentiates lethal endotoxaemia. Nature 1993; 365:756-759.
(36) Bozza M, Satoskar A B, Lin G, Lu B, Humbles A A, Gerard C, et al., Targeted disruption of Migration Inhibitory Factor gene reveals its critical role in sepsis. Journal of Experimental Medicine 1999; 189: 341-346.
(37) Bemhagen J, Bacher M, Calandra T, Metz C N, Doty S B, Donnelly T, et al. An essential role for macrophage migration inhibitory factor in the tuberculin delayed-type hypersensitivity reaction. Journal of Experimental Medicine 1996;183:277-282.
(38) Mikulowska A, Metz C N, Bucala R, Holmdahl R. Macrophage migration inhibitory factor is involved in the pathogenesis of collagen type II-induced arthritis in mice. Journal of Immunology 1997;158:5514-5517.
(39) Lacey D C, Sampey A V, Mitchell R, Bucala R, Santos L, Leech M, et al. Control of fibroblast-like synoviocyte proliferation by macrophage migration inhibitory factor (MIF). Arthritis & Rheumatism 2003;48:103-9.
(40) Juttner S, Bemnhagen J, Metz C N, Rollinghoff M, Bucala R, Gessner A. Migration inhibitory factor induces killing of Leishrnania major by macrophages: dependence on reactive nitrogen intermediates and endogenous TNF. J. Immunol. 1998;161:2383-2390.
(41) Santos L L, Morand E F, Holdsworth S R. Suppression of adjuvant arthritis and synovial macrophage inducible nitric oxide by N-iminoethyl-1-omithine, a nitric oxide synthase inhibitor. Inflammation 1997;21:299-311.

1. A method of inhibiting cytokine or biological activity of MIF comprising contacting MIF with a cytokine or biological activity inhibiting effective amount of a compound of formula (I), or a pharmaceutically acceptable salt or prodrug thereof 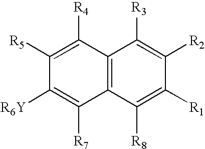
wherein
Y is O, NR₉ or S(O)_{q},
R₁ is selected from hydrogen, C₁₋₆alkyl, —(CR₁₀R_{10′})ₙhalo, —(CR₁₀R_{10′})ₙOR₁₁, —(CR₁₀R_{10′})ₙ—SR₁₁, —(CR₁₀R_{10′})ₙ—N(R₁₂)₂, —(CR₁₀R_{10′})ₙS(O)R₁₁, —(CR₁₀R_{10′})ₙS(O)₂R₁₁, —(CR₁₀R_{10′})ₙ—S(O)₃R₁₁, —(CR₁₀R_{10′})ₙC(O)R₁₃, —(CR₁₀R_{10′})ₙ—C(═NR₁₄)R₁₅ or —(CR₁₀R_{10′})ₙR₁₆;
R₂ is selected from hydrogen, C₁₋₂₀alkyl, C₂₋₂₀alkenyl, C₂₋₂₀alkynyl, —(CR₁₀R_{10′})ₘOR₁₇, —(CR₁₀R_{10′})ₘSR₁₇, —(CR₁₀R_{10′})ₘNR₁₈R₁₉, —(CR₁₀R_{10′})ₘS(O)R₂₀, (CR₁₀R_{10′})ₘS(O)₂R₂₀, —(CR₁₀R_{10′})ₘC(O)R₂₀, —(CR₁₀R_{10′})ₘC(S)R₂₀, —(CR₁₀R_{10′})ₘC(═NR₁₁)R₁₅ or —(CR₁₀R_{10′})ₘR₁₆;
R₃, R₄ and R₅ are independently selected from hydrogen, C₁₋₃alkyl, —(CR₁₀R_{10′})ₙN(R)₄)₂, —(CR₁₀R)ₙOR₁₄, —(CR₁₀R_{10′})ₙ—SR₁₄ or —(CR₁₀R_{10′})ₙhalo;
R₆ is selected from hydrogen, C₁₋₆alkyl, —C(O)C₁₋₆alkyl, —C(O)N(R₉)₂—, —C(S)N(R₉)₂—, —(CR₁₀R_{10′})ₙR₂₁, or R₆Y and R₅ together may form —X—(CH₂)ᵣ-Z-, where X and Z may be independently selected from O, S or NR₁₄;
R₇ and R₈ are independently selected from hydrogen, C₁₋₃alkyl, C₂₋₃alkenyl, C₂₋₃alkynyl or —(CR₁₀R_{10′})ₙR₂₂;
Each R₉ is independently selected from H or C₁₋₆alkyl;
Each R₁₀ and R_{10′} is independently selected from hydrogen, C₁₋₆alkyl, C₂₋₆alkenyl, C₂₋₆alkynyl, halogen, OR₁₁, SR₁₁, C₁₋₃alkoxy, CO₂R₁₄, N(R₁₄)₂, —CN, NO₂, aryl or heterocyclyl;
R₁₁ is hydrogen or C₁₋₆alkyl;
Each R₁₂ is independently selected from hydrogen, C₁₋₆alkyl, NH—C(═NR₁₄)R₁₅, C(O)R₁₄ or C(S)R₁₄;
R₁₃ is hydrogen, C₁₋₆alkyl, OR₁₄, SR₁₄ or N(R₁₄)₂;
Each R₁₄ is independently selected from hydrogen or C₁₋₃alkyl;.
R₁₅ is C₁₋₆alkyl, NH₂, NH(C₁₋₃alkyl) or N(C₁₋₃alkyl)₂, OR₂₃ or SR₂₃;
R₁₆ is hydroxy, C₁₋₃alkoxy, SH, SC₁₋₃alkyl, halo, C(O)R₃₁, C(R₂₄)₃, CN, aryl or heterocyclyl;
R₁₇ is selected from hydrogen, C₁₋₂₀alkyl, C₂₋₂₀alkenyl, C₂₋₂₀alkynyl, (CR₂₆R_{26′})ₛ—R ₂₇, C(O)R₂₅, CO₂R₂₅, C(S)R₂₅, C(S)OR₂₅, S(O)R_{25,} S(O)₂R₂₅, [C(O)CH(R₂₉)NH]ᵣ—R₂₃ or [sugar]ᵣ;
R₁₈ and R₁₉ are independently selected from hydrogen, C₁₋₂₀alkyl, C₂₋₂₀alkenyl, C₂₋₂₀alkynyl, (CR₂₆R_{26′})ₛR₂₇, C(O)R₂₅, C(S)R₂₅, S(O)R₂₅, S(O)₂R₂₅, [C(O)CH(R₂₉)NH]ᵣ—R₂₃, [sugar]ᵣ, C(═NR₂₃)NH₂ or NH—C(═NR₂₃)NH₂;
R₂₀ is selected from hydrogen, C₁₋₂₀alkyl, C₂₋₂₀alkenyl, C₂₋₂₀alkynyl, OR₂₈, SR₂₈, N(R₂₈)₂, [NH—CHR₂₉C(O)]ᵣ—OR₂₃, [sugar]ᵣ or (CR₂₆R_{26′})ₛR₂₇;
R₂₁ is OR₂₈, SR₂₈, halo or N(R₂₅)₂;
R₂₂ is halo, CO₂H, SO₃H, NO₂, NH₂, CO₂C₁₋₃alkyl, SO₃C₁₋₃alkyl or C(R₂₄)₃;
R₂₃ is hydrogen or C₁₋₃alkyl;
Each R₂₄ is independently selected from hydrogen, Cl or F;
Each R₂₅ is independently selected from hydrogen, C₁₋₂₀alkyl, C₂₋₂₀alkenyl, C₂₋₂₀alkynyl, aryl or (CR₂₆R_{26′})ₛR₂₇;
Each R₂₆ and R_{26′} is independently selected from hydrogen, C₁₋₆alkyl, C₂₋₆alkenyl, C₂₋₆alkynyl, halogen, hydroxy, C₁₋₃alkoxy, CO₂H, CO₂C₁₋₃alkyl, NH₂, NH(C₁₋₃alkyl), N(C₁₋₃alkyl)₂, CN, NO₂, aryl or heteroaryl;
R₂₇ is hydroxy, C₁₋₃alkoxy, SH, SC₁₋₃alkyl, halo, NH₂, NH(C₁₋₃alkyl), N(C₁₋₃alkyl)₂, C(O)R₃₁, aryl or heterocyclyl;
Each R₂₈ is independently selected from hydrogen, C₁₋₂₀alkyl, C₂₋₂₀alkenyl, C₂₋₂₀alkynyl or (CR₂₆R_{26′})R₃₀;
R₂₉ is the characterising group of an amino acid;
R₃₀ is halogen, hydroxy, C₁₋₃alkoxy, NH₂, NH(C₁₋₃alkyl), N(C₁₋₃alkyl)₂, C(O)R₃₁, aryl or heterocyclyl;
R₃₁ is C₁₋₃alkyl, OH, C₁₋₃alkoxy, aryl, aryloxy, heterocyclyl or heterocyclyloxy;
q is 0, 1, 2 or 3;
n is 0, 1, 2 or 3;
m is 0 or 1 to 20;
r is b 1 to 5;
s is 1 to 10; and
t is 1 or 2;
wherein an alkyl, alkenyl, alkynyl, alkyloxy, aryl or heterocyclyl group may be optionally substituted one or more times.

2. A method according to claim 1 wherein Y is O, NH, NC₁₋₆alkyl, or S(O)_{q} wherein q is 0, 1, 2 or 3.

3. A method according to claim 1 wherein R₁ is hydrogen, C₁₋₆alkyl, (CH₂)ₙOH, (CH₂)ₙNH₂, (CH₂)ₙSH, (CH₂)ₙCF₃, (CH₂)ₙCO₂H, (CH₂)ₙCO₂C₁₋₃alkyl, (CH₂)ₙC(O)NH₂, (CH₂)ₙC(O)NHC₁₋₃alkyl, (CH₂)ₙC(O)N(C₁₋₃alkyl)₂, (CH₂)ₙSO₃H or (CH₂)ₙSO₃C₁₋₃alkyl, where n is 0, 1, 2 or 3.

4. A method according to claim 1 wherein R₂ is selected from C₂₋₂₀alkyl, C₁₋₂₀alkenyl, (CR₁₀R_{10′})ₘOH, (CR₁₀R_{10′})ₘOC₁₋₂₀alkyl, (CR₁₀R_{10′})ₘOC₂₋₂₀alkenyl, (CR₁₀R_{10′})ₘOC(O)C₁₋₂₀alkyl, (CR₁₀R_{10′})ₘOC(O)C₂₋₂₀alkenyl, (CR₁₀R_{10′})ₘOC(O)aryl, (CR₁₀R_{10′})ₘO[C(O)CH(R₂₉)NH]ᵣ—H, (CR₁₀R_{10′})ₘO[sugar]ᵣ, (CR₁₀R_{10′})ₘNHC₁₋₂₀alkyl, (CR₁₀R_{10′})ₘN(C₁₋₂₀alkyl)₂, (CR₁₀R_{10′})ₘNHC₂₋₂₀alkenyl, (CR₁₀R_{10′})ₘN(C₂₋₂₀alkenyl)₂, (CR₁₀R_{10′}mN(C₁₋₂₀alkyl)(C₂₋₂₀alkenyl), (CR₁₀R_{10′})ₘNHC(O)C₁₋₂₀alkyl, (CR₁₀R_{10′})ₘNHC(O)C₂₋₂₀alkenyl, (CR₁₀R_{10′})ₘNHC(O)aryl, (CR₁₀R_{10′})ₘNH[C(O)CH(R₂₉)NH]ᵣ—H, (CR₁₀R_{10′})ₘNH-[sugar]ᵣ, (CR₁₀R_{10′})ₘSO₃H, (CR₁₀R_{10′})ₘSO₃C₁₋₂₀alkyl, (CR₁₀R_{10′})ₘSO₃C₂₋₂₀alkenyl, (CR₁₀R_{10′})ₘC(O)C₁₋₂₀alkyl, (CR₁₀R_{10′})ₘC(O)C₂₋₂₀alkenyl, (CR₁₀R_{10′})ₘCO₂H, (CR₁₀R_{10′})ₘCO₂C₁₋₂₀alkyl, (CR₁₀R_{10′})ₘCO₂C₂₋₂₀alkenyl, (CR₁₀R_{10′})ₘC(O)NHC₁₋₂₀alkyl, (CR₁₀R_{10′})ₘC(O)N(C₁₋₂₀alkyl)₂, (CR₁₀R_{10′})ₘC(O)NHC₂₋₂₀alkenyl, (CR₁₀R_{10′})ₘC(O)N(C₂₋₂₀alkenyl)₂(CR₁₀R_{10′})mC(O)N(C₁₋₂₀alkyl)(C₂₋₂₀alkenyl), (CR₁₀R_{10′})ₘC(O)[NHCH(R₂₉)C(O)]ᵣ—OH, (CR₁₀R_{10′})ₘC(O)[sugar]ᵣ, (CR₁₀R_{10′})ₘhalo, (CR₁₀R_{10′})ₘCN, (CR₁₀R_{10′})ₘheterocyclyl, (CR₁₀R_{10′})ₘaryl, (CR₁₀R_{10′})ₘNHC(═NH)NH₂, (CR₁₀R_{10′})ₘSO₂NHC₁₋₂₀alkyl, (CR₁₀R_{10′})ₘC(O)O(CH₂)₁₋₁₀CO₂H or (CR₁₀R_{10′})ₘC(O)O(CH₂)₁₋₁₀CO₂C₁₋₃alkyl; wherein each R₁₀ and R_{10′} is independently selected from hydrogen, C₁₋₆alkyl, C₂₋₆alkenyl, C₂₋₆alkynyl, halogen, OH, OC₁₋₆alkyl, CO₂H, CO₂C₁₋₃alkyl, NH₂, NHC₁₋₃alkyl, —N(C₁₋₃alkyl)₂, CN, NO₂, aryl or heterocyclyl; R₂₉ is the characterising group of an amino acid, m is 0 or an integer from 1 to 20 and r is an integer from 1 to 5;

5. A method according to claim 1 wherein R₃ is selected from hydrogen, halo, NH₂, OH, OC₁₋₃alkyl, SH or SC₁₋₃alkyl.

6. A method according to claim 1 wherein P4 is selected from hydrogen, halogen, C₁₋₃alkyl, (CH₂)ₙNH₂, (CH₂)ₙNHC₁₋₃alkyl, (CH₂)ₙNH(C₁₋₃alkyl)₂, (CH₂)ₙOH or (CH₂)ₙOC₁₋₃alkyl and n is 0, 1, 2 or 3.

7. A method according to claim 1 wherein R₅ is selected from hydrogen, halogen, (CH₂)ₙNH₂, (CH₂)ₙOH, (CH₂)ₙOC₁₋₃alkyl, (CH₂)ₙSH or (CH₂)ₙSC₁₋₃alkyl and n is 0, 1, 2 or 3.

8. A method according to claim 1 wherein R₆ is selected from hydrogen, C₁₋₃alkyl, C(O)C₁₋₃alkyl, C(O)NH(C₁₋₃alkyl), C(O)N(C₁₋₃alkyl)₂, C(S)NH(C₁₋₃alkyl) or C(S)N(C₁₋₃alkyl)₂.

9. A method according to claim 1 wherein R₅ and R₆Y taken together form —X—(CH₂)ₜ-Z- wherein X and Z are independently selected from O and S and t is 1 or 2.

10. A method according to claim 1 wherein R₇ is selected from hydrogen, C₁₋₃alkyl, (CH₂)ₙSO₃H, (CH₂)ₙNO₂, (CH₂)ₙOH, (CH₂)ₙCO₂H, (CH₂)ₙNH₂, (CH₂)ₙhalo, (CH₂)ₙCH₂halo, (CH₂)ₙCH(halo)₂ or (CH₂)ₙC(halo)₃ and n is 0, 1, 2 or 3.

11. A method according to claim 1 wherein R₈ is selected from hydrogen, C₁₋₃alkyl, or (CH₂)ₙR_{22,} wherein R₂₂ is halo, CH₂halo, CH(halo)₂ or C(halo)₃ and n is 0, 1, 2 or 3.

12. A method according to claim 1 wherein at least one of R₁₀ and R_{10′} in each (CR₁₀R_{10′}) is hydrogen.

13. A method according to claim 1 wherein at least one of R₂₆ and R_{26′} in each (CR₂₆R_{26′}) is hydrogen.

14. A method according to claim 1 wherein
Y is O, NR₉ or S(O)_{q};
R₁ is hydrogen, C₁₋₆alkyl, —(CH₂)ₙC(O)R₁₃, —(CH₂)ₙS(O)₃R₁₁, —(CH₂)ₙNH₂, —(CH₂)ₙOH, —(CH₂)ₙ—SH or —(CH₂)ₙCF₃, where R₁₁ and R₁₃ are defined in claim 1;
R₂ is selected from hydrogen, C₁₋₂₀alkyl, C₂₋₂₀alkenyl, C₂₋₂₀alkynyl, —(CR₁₀R_{10′})ₘOR₁₇, —(CR₁₀R_{10′})ₘSR₁₇, —(CR₁₀R_{10′})ₘNR₁₈R₁₉, —(CR₁₀R_{10′})ₘS(O)R₂₀, (CR₁₀R_{10′})ₘS(O)₂R₂₀, —(CR₁₀R_{10′})ₘC(O)R₂₀, —(CR₁₀R_{10′})ₘC(S)R₂₀, —(CR₁₀R_{10′})ₘC(═NR₁₁)R₁₅ or —(CR_{10 R}_{10′})ₘR₁₆, where m, R₁₀, R_{10′}, R₁₁, R₁₅, R₁₆, R₁₇, R₁₈, R₁₉, R₂₀ are as defined in claim 1;
R₃ is selected from hydrogen, halo, amino, OH, OC₁₋₃alkyl or SH;
R₄ is selected from hydrogen, halogen, C₁₋₃alkyl, (CH₂)ₙNH₂, (CH₂)ₙNHC₁₋₃alkyl, (CH₂)ₙNH(C₁₋₃alkyl)₂, (CH₂)ₙOH or (CH₂)ₙOC₁₋₃alkyl;
R₅ is selected from hydrogen, halogen, (CH₂)ₙNH₂, (CH₂)ₙOH, (CH₂)ₙOC₁₋₃alkyl, (CH₂)ₙSH or (CH₂)ₙSC₁₋₃alkyl;
R₆ is hydrogen, C₁₋₃alkyl, CH₂halo, C(O)NH(C₁₋₃alkyl), C(O)N(C₁₋₃alkyl)₂, C(S)NH(C₁₋₃alkyl) or C(S)N(C₁₋₃alkyl)₂, CH₂OH or CH₂SH;
or R₅ and YR₆ together form X—(CH₂)ₜ-Z wherein X and Z are independently selected from O and S;
R₇ is selected from hydrogen, C₁₋₃alkyl, or (CH₂)ₙSO₃H, (CH₂)ₙNO₂, (CH₂)ₙOH, (CH₂)ₙCO₂H, (CH₂)ₙNH₂, (CH₂)ₙhalo, (CH₂)ₙCH₂halo, (CH₂)ₙCH(halo)₂ or (CH₂)ₙC(halo)₃,
R₈ is hydrogen, C₁₋₃alkyl or (CH₂)ₙhalo, and
q and n are 0, 1, 2 or 3.

15. A method according to claim 1 wherein
Y is O, NR₉ or S(O)_{q};
R₁ is hydrogen, (CH₂)ₙCO₂H, (CH₂)ₙCO₂C₁₋₃alkyl, (CH₂)ₙSO₃H, (CH₂)ₙNH₂, C₁₋₃alkyl, (CH₂)ₙOH or (CH₂)ₙCF₃;
R₂ is selected from hydrogen, C₁₋₂₀alkyl, C₂₋₂₀alkenyl, C₂₋₂₀alkynyl, —(CR₁₀R_{10′})ₘOR₁₇, —(CR₁₀R_{10′})ₘSR₁₇, —(CR₁₀R_{10′})ₘNR₁₈R₁₉, —(CR₁₀R_{10′})ₘS(O)R₂₀, —(CR₁₀R_{10′})ₘS(O)₂R₂₀, —(CR₁₀R_{10′})ₘC(O)R₂₀, —(CR₁₀R_{10′})ₘC(S)R₂₀, —(CR₁₀R_{10′})ₘC(═NR₁₁)R₁₅ or —(CR₁₀R_{10′})ₘR₁₆, where m, R₁₀, R_{10′}, R₁₁, R₁₅, R_{16,} R₁₇, R₁₈, R₁₉, R₂₀ are as defined in claim 1;
R₃ is selected from hydrogen, OH or OC₁₋₃alkyl,
R₄ is selected from hydrogen, C₁₋₃alkyl, (CH₂)ₙNH₂, (CH₂)ₙOH or (CH₂)ₙOC₁₋₃alkyl;
R₅ is hydrogen, (CH₂)ₙOH or (CH₂)ₙOC₁₋₃alkyl;
R₆ is hydrogen, C₁₋₃alkyl, CH₂halo, C(O)NH(C₁₋₃alkyl), C(O)N(C₁₋₃alkyl₂, C(S)NH(C₁₋₃alkyl) or C(S)N(C₁₋₃alkyl)₂, CH₂OH or CH₂SH;
or R₅ and R₆ are taken together to form —O—(CH₂)ₜ—O where t is 1 or 2;
R₇ is selected from hydrogen, (CH₂)ₙSO₃H, (CH₂)ₙNO₂, (CH₂)ₙNH₂, or (CH₂)ₙhalo
R₈ is hydrogen, CH₃, CF₃ or CCl₃;
and q and n are 0, 1, 2 or 3.

16. A method according to claim 1 wherein
Y is O, NR₉ or S(O)_{q};
R₁ is hydrogen, (CH₂)ₙCO₂H, (CH₂)ₙCO₂C₁₋₃alkyl, (CH₂)ₙSO₃H, (CH₂)ₙNH₂, C₁₋₃alkyl, (CH₂)ₙOH or (CH₂)ₙCF₃;
R₂ is selected from hydrogen, C₁₋₂₀alkyl, C₂₋₂₀alkenyl, —(CR₁₀R_{10′})ₘOH, —(CR₁₀R_{10′})ₘNHC₁₋₂₀alkyl, —(CR₁₀R_{10′})ₘNH[C(O)CH(R₂₉)NH]—H, —(CR₁₀R_{10′})ₘSO₃H, —(CR₁₀R_{10′})ₘSO₃C₁₋₂₀alkyl, —(CR₁₀R_{10′})ₘC(O)C₁₋₂₀alkyl, —(CR₁₀R_{10′})ₘCO₂H, —(CR₁₀R_{10′})ₘCO₂C₁₋₂₀alkyl, —(CR₁₀R_{10′})ₘCN, —(CR₁₀R_{10′})ₘhalo, —(CR₁₀R_{10′})ₘaryl, —(CR₁₀R_{10′})ₘheterocyclyl, —(CR₁₀R_{10′})ₘNHC(═NH)NH₂, —(CR₁₀R_{10′})ₘSO₂NHC₁₋₂₀alkyl, CO₂(CH₂)₁₋₁₀CO₂H or CO₂(CH₂)₁₋₁₀CO₂C₁₋₃alkyl, where m, R₁₀ and R_{10′} are as defined in claim 1;
R₃ is selected from hydrogen, OH or OC₁₋₃alkyl,
R₄ is selected from hydrogen, C₁₋₃alkyl, (CH₂)ₙNH₂, (CH₂)ₙOH or (CH₂)ₙOC₁₋₃alkyl;
R₅ is hydrogen, (CH₂)ₙOH or (CH₂)ₙOC₁₋₃alkyl;
R₆ is hydrogen, C₁₋₃alkyl, CH₂halo, C(O)NH(C₁₋₃alkyl), C(O)N(C₁₋₃alkyl)₂, C(S)NH(C₁₋₃alkyl) or C(S)N(C₁₋₃alkyl)₂, CH₂OH or CH₂SH;
or R₅ and R₆ are taken together to form —O—(CH₂ₜ—rO where t is 1 or 2;
R₇ is selected from hydrogen, (CH₂)ₙSO₃H, (CH₂)ₙNO₂, (CH₂)ₙNH₂, or (CH₂)ₙhalo;
R₈ is hydrogen, CH₃, CF₃ or CCl₃;
and q and n are 0, 1, 2 or 3.

17. A method according to claim 1 wherein the compound of formula (I) is a compound of formula (II): 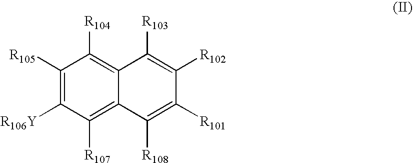
wherein Y is selected from —O—, —NH—, —NC₁₋₃alkyl- or —S(O)_{q};
R₁₀₁ is selected hydrogen, C₁₋₆alkyl, CO₂H or CO₂C₁₋₆alkyl;
R₁₀₂ is selected from C₁₋₂₀alkyl, C₂₋₂₀alkenyl, CO₂H, CO₂C₁₋₂₀alkyl, CO₂C₂₋₂₀alkenyl, CO₂(CH₂)ₘR₁₀₉, SO₃H, SO₃C₁₋₂₀alkyl, SO₃C₂₋₂₀alkenyl, SO₃(CH₂)ₘR₁₀₉, C(O)C₁₋₂₀alkyl or (CH₂)ₘR₁₁₀;
R₁₀₃ is selected from hydrogen, hydroxy, methoxy or C₁₋₃alkyl;
R₁₀₄ is selected from hydrogen, C₁₋₃alkyl, NH₂, NH(C₁₋₃alkyl), N(C₁₋₃alkyl)₂ or (CH₂)ₙOH;
R₁₀₅ is selected from hydrogen, (CH₂)ₙOH or (CH₂)ₙOC₁₋₃alkyl;
R₁₀₆ is selected from hydrogen, C₁₋₃alkyl, C(O)NH₂, C(O)NH(C₁₋₃alkyl), C(O)N(C₁₋₃alkyl)₂, C(S)NH₂, C(S)NH(C₁₋₃alkyl) or C(S)N(C₁₋₃alkyl)₂;
R₁₀₇ is selected from hydrogen, hydroxy, halo, amino, nitro, cyano, SO₃H or CO₂H;
R₁₀₈ is selected from hydrogen or methyl;
R₁₀₉ is selected from halogen, hydroxy, C₁₋₃alkoxy, NH₂, NH(C₁₋₃alkyl), N(C₁₋₃alkyl)₂, CO₂H or CO₂C₁₋₃alkyl;
R₁₁₀ is selected from hydroxy, C₁₋₃alkyl, halo, CO₂H, CO₂C₁₋₃alkyl, CN, NH₂, NH(C₁₋₃alkyl) or N(C₁₋₃alkyl)₂;
n is 0 or an integer from 1 to 3;
m is 0 or an integer from 1 to 20; and
wherein an alkyl, alkenyl or alkyloxy, group may be optionally substituted one or more times.

18. A method according to claim 1 wherein the compound of formula (I) is selected from the group consisting of:
6,7-dihydroxy-2-naphthalene
6,7-dimethoxy-2-naphthalene
6,7-dimethoxy-2-acetonoaphthone
6,7-Dimethoxy-2-naphthoic acid
2-carboxy-6-hydroxynaphthalene-5-sulfonic acid
6,7-dihydroxy-2-naphthalenesulfonic acid
Pentyl 6,7-dihydroxy-2-naphthalenesulfonate
6-hydroxy-2-naphthalenesulfonic acid
6-methylamino-2-naphthalenesulfonic acid
2,3-dihydronaphtho[2,3-b][1,4]dioxine-7-carboxylic acid
Methyl 6-hydroxy-2-naphthoate
dodecanyl-6-hydroxy-2-naphthoate
[(6-hydroxy-2-naphthyl)carbonyl]oxyhexanoic acid
(6-methoxy-6-oxohexyl)-6-hydroxy-2-naphthoate
6-hydroxy-5-nitro-2-naphthoic acid
Ethyl 1,6-dihydroxy-2-naphthoate
Ethyl 6-[(dimethylamino)carbonyl]sulfanyl-1-methoxy-2-naphthoate
Ethyl 6-hydroxy-1-methoxy-2-naphthoate
Ethyl 6-[(dimethylamino)thiocarbonyl]oxy-1-methoxy-2-naphthoate
7-methoxy-3-hydroxy-2-naphthoic acid
Methyl 7-methoxy-3-hydroxy-2-naphthoate
Methyl 7-methoxy-3-methyl-2-naphthoate
7-methoxy-3-methyl-2-naphthoic acid
5-bromo-6-methoxy-2-methyl-3-naphthoic acid
6-hydroxy-[2-(1-pentylamino)methyl]-3-naphthoic acid
Methyl 3-bromomethyl-7-hydroxy-2-naphthoate
Methyl 7-methoxy-2-naphthoate
Methyl 7-hydroxy-2-naphthoate
Methyl 7-hydroxy-8-nitro-2-naphthoate
Methyl 6-hydroxy-5-nitro-2naphthoate
Methyl 6-methoxy-5-nitro-2-naphthoate
Methyl 5-amino-6-methoxy-2-naphthoate
Methyl 6-methoxy-2-naphthoate
2-hydroxymethyl-6-methoxynaphthalene
2-bromomethyl-6-methoxy-naphthalene
2-cyanomethyl-6-methoxynaphthalene
2-(1-cyano-1-hex-5-enyl)-6-methoxynaphthalene
2-(6-methoxy-2-naphthyl)hept-6-enoic acid
Methyl 2-(6-methoxy-2-naphthyl)hept-6-enoate
7-hydroxy-2-(6-methoxy-2-naphthyl)heptanoic acid
Methyl 6-methoxy-8-methyl-2-naphthoate ester
6-hydroxy-2-naphthanoic acid
6-methoxy-α-methyl-2-naphthalene acetic acid
2,6-naphthalene disulfonic acid.

19. A method of treating, preventing or diagnosing a disease or condition wherein MIF cytokine or biological activity is implicated comprising the administration of a treatment, prevention or diagnostic effective amount of a compound of formula (I) as defined in claim 1 or a pharmaceutically acceptable salt or prodrug thereof to a subject in need thereof.

20. A method according to claim 19 wherein the disease or condition is selected from autoimmune diseases, solid or haemopoeitic tumours, or chronic or acute inflammatory diseases.

21. A method according to claim 19 wherein the disease or condition selected from the group comprising rheumatic diseases, spondyloarthropathies, crystal arthropathies, Lyme disease, connective tissue diseases, vasculitides, glomerulonephritis, interstitial nephritis, inflammatory bowel disease, peptic ulceration, gastritis, oesophagitis, liver disease, autoimmune diseases, pulmonary diseases, cancers whether primary or metastatic, atherosclerosis, disorders of the hypothalamic-pituitary-adrenal axis, brain disorders, corneal disease, iritis, iridocyclitis, cataracts, uveitis, sarcoidosis, diseases characterised by modified angiogenesis, endometrial function, psoriasis, endotoxic (septic) shock, exotoxic (septic) shock, infective (true septic) shock, other complications of infection, pelvic inflammatory disease, transplant rejection, allergies, allergic rhinitis, bone diseases, atopic dermatitis, UV(B)-induced dermal cell activation, malarial complications, diabetes mellitus, pain, inflammatory consequences of trauma or ischaemia, testicular dysfunctions and wound healing.

22. A method according to claim 21 wherein the disease or condition is selected from the group consisting of rheumatoid arthritis, osteoarthritis, psoriatic arthritis, ankylosing spondylitis, reactive arthritis, Reiter's syndrome, gout, pseudogout, calcium pyrophosphate deposition disease, systemic lupus erythematosus, systemic sclerosis; polymyositis, dermatomyositis, Sjögren's syndrome, polyarteritis nodosa, Wegener's granulomatosis, Churg-Strauss syndrome, ulcerative colitis, Crohn's disease, cirrhosis, hepatitis, diabetes mellitus, thyroiditis, myasthenia gravis, sclerosing cholangitis, primary biliary cirrhosis, diffuse interstitial lung diseases, pneumoconioses, fibrosing alveolitis, asthma, bronchitis, bronchiectasis, chronic obstructive pulmonary disease, adult respiratory distress syndrome, colon cancer, lymphoma, lung cancer, melanoma, prostate cancer, breast cancer, stomach cancer, leukemia, cervical cancer and metastatic cancer, ischaemic heart disease, myocardial infarction, stroke, peripheral vascular disease, Alzheimer's disease, multiple sclerosis, diabetic retinopathy, parturition, endometriosis, osteoporosis, Paget's disease, sunburn and skin cancer.

23. A method according to claim 19 wherein the subject is a human subject.

24. A pharmaceutical composition comprising a compound of formula (I) as defined in claim 1 or a pharmaceutically acceptable salt or prodrug thereof and a pharmaceutically acceptable carrier, diluent or excipient.

25. A pharmaceutical composition according to claim 24 further comprising a glucocorticoid.

26. A method of treating or preventing a disease or condition wherein MIF cytokine or biological activity is implicated comprising administering to a mammal a compound of formula (I) as defined in claim 1 or a pharmaceutically acceptable salt or prodrug thereof and a second therapeutic agent.

27. A method according to claim 26 wherein the second therapeutic agent is a glucocorticoid.

28. A method of prophylaxis or treatment of a disease or condition for which treatment with a glucocorticoid is indicated, said method comprising administering to a mammal a glucocorticoid and a compound of formula (I) as defined in claim 1 or a pharmaceutically acceptable salt or prodrug thereof.

29. A method of treating steroid-resistant diseases comprising administering to a mammal a glucocorticoid and a compound of formula (I) as defined in claim 1 or a pharmaceutically acceptable salt or prodrug thereof.

30. A method of enhancing the effect of a glucocorticoid in mammals comprising administering a compound of formula (I) as defined in claim 1 or a pharmaceutically acceptable salt or prodrug thereof, simultaneously, separately or sequentially with said glucocorticoid.

31. A compound of formula (U) or a pharmaceutically acceptable salt or prodrug thereof: 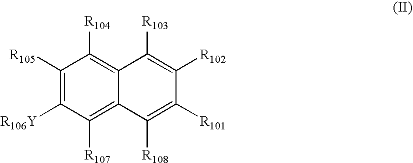
wherein Y is selected from —O—, —NH—, —NC₁₋₃alkyl- or —S(O)_{q};
R₁₀₁ is selected hydrogen, C₁₋₆alkyl, CO₂H or CO₂C₁₋₆alkyl;
R₁₀₂ is selected from C₁₋₂₀alkyl, C₂₋₂₀alkenyl, CO₂H, CO₂C₁₋₂₀alkyl, CO₂C₂₋₂₀alkenyl, CO₂(CH₂)ₘR₁₀₉, SO₃H, SO₃C₁₋₂₀alkyl, SO₃C₂₋₂₀alkenyl, SO₃(CH₂)ₘR₁₀₉, C(O)C₁₋₂₀alkyl or (CH₂)ₘR₁₁₀;
R₁₀₃ is selected from hydrogen, hydroxy, methoxy or C₁₋₃alkyl;
R₁₀₄ is selected from hydrogen, C₁₋₃alkyl, NH₂, NH(C₁₋₃alkyl), N(C₁₋₃alkyl)₂ or (CH₂)ₙOH;
R₁₀₅ is selected from hydrogen, (CH₂)ₙOH or (CH₂)ₙOC₁₋₃alkyl;
R₁₀₆ is selected from hydrogen, C₁₋₃alkyl, C(O)NH₂, C(O)NH(C₁₋₃alkyl), C(O)N(C₁₋₃alkyl)₂, C(S)NH₂, C(S)NH(C₁₋₃alkyl) or C(S)N(C₁₋₃alkyl)₂;
R₁₀₇ is selected from hydrogen, hydroxy, halo, amino, nitro, cyano, SO₃H or CO₂H;
R₁₀₈ is selected from hydrogen or methyl;
R₁₀₉ is selected from halogen, hydroxy, C₁₋₃alkoxy, NH₂, NH(C₁₋₃alkyl), N(C₁₋₃alkyl)₂, CO₂H or CO₂C₁₋₃alkyl;
R₁₁₀ is selected from hydroxy, C₁₋₃alkyl, halo, CO₂H, CO₂C₁₋₃alkyl, CN, NH₂, NH(C₁₋₃alkyl) or N(C₁₋₃alkyl)₂;
n is 0 or an integer from 1 to 3;
m is 0 or an integer from 1 to 20; and
wherein an alkyl, alkenyl or alkyloxy, group may be optionally substituted one or more times.

32. A compound according to claim 31 wherein Y is selected from —O—, —S—, —NH—or SO₃.

33. A compound according to claim 31 wherein R₁₀ is selected from hydrogen, CO₂H or CO₂C₁₋₃alkyl.

34. A compound according to claim 31 wherein R₁₀₂ is selected from from C₁₋₂₀alkyl, C₂₋₂₀alkenyl, CO₂H, CO₂C₁₋₂₀alkyl, CO₂C₂₋₂₀ alkenyl, CO₂(CH₂)ₘCO₂H, SO₃C₁₋₂₀alkyl, SO₃C₂₋₃₀alkenyl, SO₃(CH₂)ₘCO₂H, (CH₂)ₘhydroxy, (CH₂)ₘNH₂, (CH₂)ₘCN or (CH₂)ₘhalo.

35. A compound according to claim 31 wherein R₁₀₃ is selected from hydrogen, hydroxy or methoxy.

36. A compound according to claim 31 wherein R₁₀₄ is selected from hydrogen, hydroxy, methyl, NH₂ or CH₂OH.

37. A compound according to claim 31 wherein R₁₀⁵ is selected from hydrogen, hydroxy or methoxy.

38. A compound according to claim 31 wherein R₁₀₆ is selected from hydrogen, C₁₋₃alkyl, C(O)NH₂, C(O)NH(C₁₋₃alkyl), C(O)N(C₁₋₃alkyl)₂, C(S)NH₂, C(S)NH(C₁₋₃alkyl) or C(S)N(C₁₋₃alkyl)₂.

39. A compound according -to claim 31 wherein R₁₀₇ is selected from hydrogen, hydroxy, halo, cyano, NH₂, nitro or SO₃H.

40. A compound according to claim 31 wherein R₁₀₈ is hydrogen.

41. A compound of formula (I) selected from the group consisting of
6,7-dimethoxy-2-acetonoaphthone
2-carboxy-6-hydroxynaphthalene-5-sulfonic acid
Pentyl 6,7-dihydroxy-2-naphthalenesulfonate
2,3-dihydronaphtho[2,3-b][1,4]dioxine-7-carboxylic acid
Methyl 6-hydroxy-2-naphthoate
dodecanyl-6-hydroxy-2-naphthoate
[(6-hydroxy-2-naphthyl)carbonyl]oxyhexanoic acid
(6-methoxy-6-oxohexyl)-6-hydroxy-2-naphthoate
6-hydroxy-5-nitro-2-naphthoic acid
Ethyl 1,6-dihydroxy-2-naphthoate
Ethyl 6-[(dimethylamino)carbonyl]sulfanyl-1-methoxy-2-naphthoate
Ethyl 6-hydroxy-1-methoxy-2-naphthoate
Ethyl 6-[(dimethylamino)thiocarbonyl]oxy-1-methoxy-2-naphthoate
7-methoxy-3-hydroxy-2-naphthoic acid
Methyl 7-methoxy-3-hydroxy-2-naphthoate
Methyl 7-methoxy-3-methyl-2-naphthoate
7-methoxy-3-methyl-2-naphthoic acid
5-bromo-6-methoxy-2-methyl-3-naphthoic acid
6-hydroxy-[2-(1-pentylamino)methyl]-3-naphthoic acid
Methyl 3-bromomethyl-7-hydroxy-2-naphthoate
Methyl 7-methoxy-2-naphthoate
Methyl 7-hydroxy-2-naphthoate
Methyl 7-hydroxy-8-nitro-2-naphthoate
Methyl 6-hydroxy-5-nitro-2naphthoate
Methyl 6-methoxy-5-nitro-2-naphthoate
Methyl 5-amino-6-methoxy-2-naphthoate
Methyl 6-methoxy-2-naphthoate
2-hydroxymethyl-6-methoxynaphthalene
2-bromomethyl-6-methoxy-naphthalene
2-cyanomethyl-6-methoxynaphthalene
2-(1-cyano-1-hex-5-enyl)-6-methoxynaphthalene
2-(6-methoxy-2-naphthyl)hept-6-enoic acid
Methyl 2-(6-methoxy-2-naphthyl)hept-6-enoate
7-hydroxy-2-(6-methoxy-2-naphthyl)heptanoic acid
Methyl 6-methoxy-8-methyl-2-naphthoate ester.
